SEQUENCE LISTING (197 sequences)

SEQ ID NO: 1 - DNA, 236 bp, Oryctolagus cuniculus
  misc_feature (117)..(117): n is any nucleotide a, c, g, or t
cggaagccgcgcatcactgagaccgagtcaccttatcaggagctccagggtcagaggtcagatgtgtacagcgacctcaacacacagaggcagtattacaaatgagcccaaaccgcnacagtccacaatgggacgcctggatccagngcttcctgacggccacccagcaccccagcgtgccctccctgacacctctcccctctgttgctgccaaataaacatggagcacagaaaaa

SEQ ID NO: 2 - DNA, 57 bp, Oryctolagus cuniculus
cggaatcctgggagaggccaagccagagcaataaagagagcgcttttgcttcaaaaa

SEQ ID NO: 3 - DNA, 110 bp, Oryctolagus cuniculus
cggacggggagggagcagagaaagggtgggagaagctatggagcaaatgttttacaacctgaacctcagaactgtgatcctccaaggagagcactacttgaagacaaaaa

SEQ ID NO: 4 - DNA, 134 bp, Oryctolagus cuniculus
cggagtgggaagaggcctgggctcaagggggctggtgggcgagggggaccgaacccctccgacctccttgggaggccctgggggctggggacattttaatcatcaataaacaaagcactttattctgtgaaaaa

SEQ ID NO: 5 - DNA, 127 bp, Oryctolagus cuniculus
cggatccagcggcgccgcgcgtctgcccgcgctctcctgccagccgccaggcagcttgttcacagccctgaaccctctcccaaacctgggaccaaacggaaataaagtttcttgctgcagcgaaaaa

SEQ ID NO: 6 - DNA, 230 bp, Oryctolagus cuniculus
cggcacacgggcgcaagaagacagaacggcgttgggcaggaacgtgcaaaccatcaactgcaaagctagagctgaggcctttagacgtttatcttatcgtgtattgggggcggggaggggcgtgaacgggagagaaactcctgctatagcacaaaaacatgcctttagctgtgttttactactctttttatctttggggctagcaataaagacatttaaaatgccaaaaa

SEQ ID NO: 7 - DNA, 190 bp, Oryctolagus cuniculus
cggcccgataggtgggtgccctggctggagcttcgcttcctgtctttctccttcctgagatgggtctgagcacccaggggccccacgagacacgtagggctggcagctcccctgcttctgggggagtccaggagcctggggtagaaagtggagttttagtatttttggaataaatactaaaagctaaaaa

SEQ ID NO: 8 - DNA, 340 bp, Oryctolagus cuniculus
cggctgagtgaaggaatgatgctcattcattttgttaaccagggacacctgtgtagtgctttctgtataccaggcctggggacacctgccctcagagttgacactcttgtgtataaacctgaatttctgtacattctggtgcctgtatttgagttcacatactatgatttttccaaagtaggaagaccttgattttcctcccagtttctcaaatgatacggaaacttgaccaggaaggactgtacctccatttgtggtgaaccctgtctgccgtatgtcacacatcatgatgctctttttctctcctttctaaaaaataaagtgtggtgaaagacaaaaa

SEQ ID NO: 9 - DNA, 31 bp, Oryctolagus cuniculus
cgggcattaaacactctcacaaacacaaaaa

SEQ ID NO: 10 - DNA, 182 bp, Oryctolagus cuniculus
cgggccctccattgtccaccgcaaatgcttctaagtcacttccctgctctgtttctagcccacaactgtgaatgttttgtggaataatgccttcaattcctttccaagtcatgcctatccaaagctctgactcattacctatgtattttttttaataaatctgaaatatgctaccacaaaaa

SEQ ID NO: 11 - DNA, 164 bp, Oryctolagus cuniculus
cgggccccctccatccccagtgagggcaggagagaggccatcctcctgtcccctcccacgccatgaggccaggacgctgccctcttccgtggtgccgatgcctgtgtgtgttgtgtgtgagccacccacgtgtgagggaaataaacacccagcacagagaaaaa

SEQ ID NO: 12 - DNA, 22 bp, Oryctolagus cuniculus
cggtcagcctcccccccaaaaa

SEQ ID NO: 13 - DNA, 144 bp, Oryctolagus cuniculus
cggtcggcggcgctccgcgtagactcgccgaggttttcaaagtgttgagctcgctgcttgttctgtaacgttatacataacttatctctgaggacgacgagcccgtaatgttttcagattaaatgaagtgtgagacgccaaaaa

SEQ ID NO: 14 - DNA, 147 bp, Oryctolagus cuniculus
cggtcgtatcggcagtgactttatacacgggaggaggggtgttgcgggctggttcttgttttgacagagtttggagcttggtttgtgcatcacattctactatacaatgaggcttattttcccagaataaaataaagcaaacaaaaa

SEQ ID NO: 15 - DNA, 189 bp, Oryctolagus cuniculus
cggtggtggccaggacggagccgctcagggaaggccacggccttggggacgcctcttacctcagcttcccccttctctgcgacccccatcttttgcgtggaccctcgggggtcggggtggggtggctcctgttttcctactctggtgccgagggaggccttttccaaataaacagtttacttggaaaaa

SEQ ID NO: 16 - DNA, 342 bp, Oryctolagus cuniculus
cggtgttaccagtgcgaaaaggatgagagctttggagacagccaggggcggtgccccctgacagaccgcggaggcctcagttgtagctgggctgactgatggttctgttcctgctgcttcgtccctgggctgtgacgagtgcggagcgggccctctgagtgcagtggagtttgctgacaaggatggggcagaccataggaactcagcgtctttggcgttttcctgagcgaccatcaacaaatgcaaaaataatagaaattttgaggcccctgcatgtcagctggaaaggttatgactactaattttgtcaaaagaatttcaataaaataagtaaaccaaaaa

SEQ ID NO: 17 - DNA, 159 bp, Oryctolagus cuniculus
cggttaccagtgtcattctcccagtcagatccaacaaaatgaaatgccaggctcgtgtgctcactccagtgtgagttatctactgtacgctgtaccgtctcatgctttaagatctgtgtacctagtggaattaaacatttggcctaaaccccataaaaa

SEQ ID NO: 18 - DNA, 114 bp, Oryctolagus cuniculus
cggttcctcgctcgagccactgtactcccagcatgccctgccccagccccacccgtctctctgtgtctcgctgtgatagaacaataaatattttattttttgtcctggcaaaaa

SEQ ID NO: 19 - DNA, 112 bp, Oryctolagus cuniculus
cggtgggccttggcttctccagccacctccagtactgtgtgatgtgactctcagttgtaccttccaattctttctgacctgcattataaatgttataattttattccaaaaa

SEQ ID NO: 20 - DNA, 180 bp, Oryctolagus cuniculus
cggccgggccacgttggaagcgcttcccctcgggcgccctctctccagcgcgaacactggaatgcatatactactttatgtgctgtgttttttattcttggatacatttgattttttcacgtaagtccacatatacttctataagagcgtgacttgtaataaagggttaatgtagaaaaa

SEQ ID NO: 21 - DNA, 50 bp, Oryctolagus cuniculus
cggcggtgataaaacaataaagcagtatctttttttatgacatacaaaaa

SEQ ID NO: 22 - DNA, 159 bp, Oryctolagus cuniculus
cggtgcactagctttagtctcaattagtccaacaacagccctaattacctttattatccttattctactaacaattctagaattcgccgtagccttgatccaagcttacgtctttactctccttgtaagcctatacctacatgacaatacctacaaaaa

SEQ ID NO: 23 - DNA, 45 bp, Oryctolagus cuniculus
cggccgatgtctctctctctcactgtccactctgcctgacaaaaa

SEQ ID NO: 24 - DNA, 159 bp, Oryctolagus cuniculus
cggcggtgcctggaggaccctgcggggccagggacacctgcccgaaacgcacacacccgcggagatcaactcagtcaacttttcgtagcttggggaaaattcttccaagagtgtgtttgttacaggcctgcgccccagcttcaacgctttgcccaaaaa

SEQ ID NO: 25 - DNA, 106 bp, Oryctolagus cuniculus
  misc_feature (48)..(48): n is any nucleotide a, c, g, or t
cggttagcaaccctgaagtgattttagaatttaggatgctgtgtgtgnttcctgtttgataatctttactgaatgagaattaaatagtctgtattccacagaaaaa

SEQ ID NO: 26 - DNA, 214 bp, Oryctolagus cuniculus
cggttcacaagcacagggagatgccgtgggctgacatctgcaggccgcaagagtcgtggccttggaaagggtcacaagttccaccacaccattggtggttctcgccgtgcagcctggagaaggcgcaataccctccagctgcaccgttaccgctaactcaagtaatgtttgtaaaattcatgcctaataaaccatttaggacagtcaagaaaaa

SEQ ID NO: 27 - DNA, 96 bp, Oryctolagus cuniculus
cggagtatgatgctctggacgttgccaacaaaattgggatcatctgaacggagtccagctggctaattctaaatatatgttttttcaccgcaaaaa

SEQ ID NO: 28 - DNA, 121 bp, Oryctolagus cuniculus
cggtagctcaaaagaaagcaagcttcctcagagctcaggagcgggctgctgagagctaaaccaaacaactttctatgaagatttttctgataaagacaataaacttattgaaagccaaaaa

SEQ ID NO: 29 - DNA, 109 bp, Oryctolagus cuniculus
cggtaatatcatgtgcacatcaagaaaatgaaatattgttctcttgtggttaacatgtattattttcaatatcttaatatcctaataaagagtccataaaatccaaaaa

SEQ ID NO: 30 - DNA, 152 bp, Oryctolagus cuniculus
cgggatacgtacctggctcctgccatcggatcagtgtgatgcactggctgcagcgctctggccgcggcggccattggagggtgaaccaacagaacaaggaagacctttctctctgtctctctctctcactgtccactctgcctgtctaaaaa

SEQ ID NO: 31 - DNA, 146 bp, Oryctolagus cuniculus
cggcttacaagaccagagtaatatttatactactgggaccttcatttgaggattttgttttcgattaggcttgcgaggggtataagaataaggatggtggtgaagtagaggacagatgctacttgtccaatggtgatggacaaaaa

SEQ ID NO: 32 - DNA, 50 bp, Oryctolagus cuniculus
cgggtgcgcattttatttgtgtttaaataaaaccttgaaaacctgaaaaa

SEQ ID NO: 33 - DNA, 378 bp, Oryctolagus cuniculus
cggctgggtgctggtgtcgcctctgagcaaggaggacgcgggggagtacgagtgccacgcgtccaactcccaggggcaggcgtcggcgtccgccaagatcacagtggtggacaccttgcacgagatcccagtgcggaaaggggaaggtgctgagctgtaaccgcagcactgcaggtcggcaagctgcaagcagtcacgggaactgcggctcctgctctcggctaacacgcggccctagctaacgctttacttagccactggtttcacacaacagtgccagcataaggatgacacatcgagactatctacaaaactttatttacagaaaaacatgcgtatcattaaacaaaacaaataaaatgcttttcaccacaaaaa

SEQ ID NO: 34 - DNA, 76 bp, Oryctolagus cuniculus
cggctgcagaagcaagaacccagattgtgtcttgtggtattttaataatgcaatagaagattattgatccgaaaaa

SEQ ID NO: 35 - DNA, 215 bp, Oryctolagus cuniculus
cggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaaaaa

SEQ ID NO: 36 - DNA, 111 bp, Oryctolagus cuniculus
cggaggcaggccgagaagaacgtggagaagaagatcagcaagttcacggacgtcctcaagacccacgggctcgtggtttgagcacaataaagactttgttccttccaaaaa

SEQ ID NO: 37 - DNA, 106 bp, Oryctolagus cuniculus
cggaggtcgacaggacccagaaggaatctctcccgccgcaccttctgatcctggagccccagtgaacctgggctgcaaatataaacttttttttttccmctaaaaa

SEQ ID NO: 38 - DNA, 91 bp, Oryctolagus cuniculus
cggcagtagcagccatttggggactgaacctacagaaggaagacctttctctcagtctctctctctccctaactgcctggctaaacaaaaa

SEQ ID NO: 39 - DNA, 168 bp, Oryctolagus cuniculus
cggcgcttgacggttgtcacagctccagaacaaatcctgggagacaggcgagcgcgagtcgcagggcaggaattccacacactcgtgctgtttttgatacctgctttttgttttgttttgtaaaaatgatgaacttgagaaaataaaacgtcagtgttgacctaaaaa

SEQ ID NO: 40 - DNA, 177 bp, Oryctolagus cuniculus
cgggatcctgtcagaaggggggctggggcagaagccaccaaacaagctgccatcccctcccccgatttctgtggaccctgtggtcaggtgctgtcccttttccacaggggtgtgtgtgtgtgtgtgtgtgtacatgtgtgtgcgctccaatagagtttgtacactttcaaacaaaaa

SEQ ID NO: 41 - DNA, 111 bp, Oryctolagus cuniculus
cggttacatacaaggatcctgcctatcttaaagaccctaaagtatgtaacatgaagtatcgggagtaagctttgtaaggttaccaccaataaactttttttagtacaaaaa

SEQ ID NO: 42 - DNA, 115 bp, Oryctolagus cuniculus
cggtccgtggctcgcatcgccaagctggagaaggccaaggccaaggagctggccacgaagctggggtgaccgcgggctgcttgtacataataaagctggtttgtcccaataaaaa

SEQ ID NO: 43 - DNA, 152 bp, Oryctolagus cuniculus
cggaggagcccaagtgggtaggcagattcctacctaccagggttatttttggggagggaggactatgcatagggctgtattctctagaatctattttactaactgacctgttttgggatatgttacccaaataaaaaatgtttatacaaaaa

SEQ ID NO: 44 - DNA, 36 bp, Oryctolagus cuniculus
cgggactgtgagaaataaacggccgccctccaaaaa

SEQ ID NO: 45 - DNA, 241 bp, Oryctolagus cuniculus
cggcgggcacaggcccaaaggtctggagggggagcggcctgcgagactcacaagaggggaagccgacagagacacttacagacgaagcgctgtgccccctggtgccgacaagaaagccgaggctggggctgggttagcaactgaattccagtttagaggcggatttggtcgtggacgtggtcagccaccccagtaaagatggaggggattattttgtgttaaataaacttgtagtcaaaaa

SEQ ID NO: 46 - DNA, 143 bp, Oryctolagus cuniculus
  misc_feature (40)..(40): n is any nucleotide a, c, g, or t
cggccccgccctgcttccacggcgcccttgactgcacccnaggaatnacgcggagctcaggacagcccgccgccaaggcgtgggcagacatgtaatcgagggactgaccgatcacggaattaaattgggtgcaacttcaaaaa

SEQ ID NO: 47 - DNA, 401 bp, Oryctolagus cuniculus
cggtgctacttgagacaaaagacgcccctgcagtcttttgttcaacattgtacaatttagaattctgtccaacactaatttattttgccttgagttttactaccgagtgagactgtggaccccatatgcctgaactcaaaaaagccaaggatctgtgagccgcgctgctctcccaagacttccattcccttctgattggcacacttgcattgctcccgtgaccactctctaggtagcattccgtgtggattcccactctgctgttctccatgttagagtgaaagatttaggcactacatacaattggtaaaggaaaagcattttcttaagagttataactgtatgtaaacattgtataatgatacgaaataaaatgcacattgtaggacattctccaaaaa

SEQ ID NO: 48 - DNA, 236 bp, Oryctolagus cuniculus
cggatttccacctaccacttccctgaatgcaggaacgcctccttactgtattccacattctatcttacatagtgtaatgagacaatatcagaaataaacatgtaacgccaatgcatattcacattctcgtaggagtgtgtagagaagctgatgcctcatcgcgccattctgtcattggctgttatcatctaatgttttccgtgtatcctgacaaataaagcagcatatgacaaaaa

SEQ ID NO: 49 - DNA, 146 bp, Oryctolagus cuniculus
cggtgacattcctggcgtccgctttaaggtggtcaaagtagccaatgtgtctcttctggccttatacaagggcaagaaggagaggccaaggtcataaacgttgatggcagaaatgtattatgaataaattttcatattcctaaaaa

SEQ ID NO: 50 - DNA, 220 bp, Oryctolagus cuniculus
cgggtgaccagacaggccatgcctttcaagtgcatgcagctgactgacttcattctcaagtttccacacagtgcccgccagaaatatgtccgaaaagcctgggagaaggcagacatcaatacaaaatgggcagccacaagatgggccaagaaaattgaaggcggagaaaggaaagctaagatgacagattttgaccgttataaagtcatgaaatcaaaaa

SEQ ID NO: 51 - DNA, 73 bp, Oryctolagus cuniculus
cggagttataccacagtgattaaaatgagttctttttttttctcctaggaagatagttccccagatttaaaaa

SEQ ID NO: 52 - DNA, 96 bp, Oryctolagus cuniculus
cggattatcatgctctggacgttgccaacaaaattgggatcatctgaacggagtccagctggctaattctaaatatatgttttttcaccacaaaaa

SEQ ID NO: 53 - DNA, 127 bp, Oryctolagus cuniculus
cgggcgttgtactgcgtttggcactttacgctgaccatcggtaacggacatttatcaccggagttttttgttgttgtttgtttttttaaaaaaactattaaataaacggttattttacaggtaaaaa

SEQ ID NO: 54 - DNA, 122 bp, Oryctolagus cuniculus
cggaccacggtgccacattgcctgttgatgggagttccaagatctcagcttcctcagtgtgtccagctcttcgattgtagctaaacttgatcaacaaataaaattccaccttggcctaaaaa

SEQ ID NO: 55 - DNA, 117 bp, Oryctolagus cuniculus
cgggagaggtcctgactctctgggagcctgggctgagtggcctggatgttcccaccctcccttgctgccaggttgtggctacagctacaaataaaaaccctttattttcccgaaaaa

SEQ ID NO: 56 - DNA, 48 bp, Artificial Sequence
  Description of Artificial Sequence cDNA anchor primer
gaattcaactggaagcggccgcaggaattttttttttttttttttvnn

SEQ ID NO: 57 - DNA, 16 bp, Artificial Sequence
  Description of Artificial Sequence 5′ RT Primer
aggtcgacggtatcgg

SEQ ID NO: 58 - DNA, 16 bp, Artificial Sequence
  Description of Artificial Sequence 5′ PCR Primer
ggtcgacggtatcggn

SEQ ID NO: 59 - DNA, 15 bp, Artificial Sequence
  Description of Artificial Sequence universal 3′ PCR Primer
gagctccaccgcggt

SEQ ID NO: 60 - DNA, 16 bp, Artificial Sequence
  Description of Artificial Sequence 5′ PCR Primer
cgacggtatcggnnnn

SEQ ID NO: 61 - DNA, 16 bp, Artificial Sequence
  Description of Artificial Sequence 5′ PCR Primer with parsing bases AAGC
cgacggtatcggaagc

SEQ ID NO: 62 - DNA, 16 bp, Artificial Sequence
  Description of Artificial Sequence 5′ PCR Primer with parsing bases AATC
cgacggtatcggaatc

SEQ ID NO: 63 - DNA, 16 bp, Artificial Sequence
  Description of Artificial Sequence 5′ PCR Primer with parsing bases ACGG
cgacggtatcggacgg

SEQ ID NO: 64 - DNA, 16 bp, Artificial Sequence
  Description of Artificial Sequence 5′ PCR Primer with parsing bases AGTG
cgacggtatcggagtg

SEQ ID NO: 65 - DNA, 16 bp, Artificial Sequence
  Description of Artificial Sequence 5′ PCR Primer with parsing bases ATCC
cgacggtatcggatcc

SEQ ID NO: 66 - DNA, 16 bp, Artificial Sequence
  Description of Artificial Sequence 5′ PCR Primer with parsing bases CACA
cgacggtatcggcaca

SEQ ID NO: 67 - DNA, 16 bp, Artificial Sequence
  Description of Artificial Sequence 5′ PCR Primer with parsing bases CCCG
cgacggtatcggcccg

SEQ ID NO: 68 - DNA, 16 bp, Artificial Sequence
  Description of Artificial Sequence 5′ PCR Primer with parsing bases CTGA
cgacggtatcggctga

SEQ ID NO: 69 - DNA, 16 bp, Artificial Sequence
  Description of Artificial Sequence 5′ PCR Primer with parsing bases GCAT
cgacggtatcgggcat

SEQ ID NO: 70 - DNA, 16 bp, Artificial Sequence
  Description of Artificial Sequence 5′ PCR Primer with parsing bases GCCC
cgacggtatcgggccc

SEQ ID NO: 71 - DNA, 16 bp, Artificial Sequence
  Description of Artificial Sequence 5′ PCR Primer with parsing bases TCAG
cgacggtatcggtcag

SEQ ID NO: 72 - DNA, 16 bp, Artificial Sequence
  Description of Artificial Sequence 5′ PCR Primer with parsing bases TCGG
cgacggtatcggtcgg

SEQ ID NO: 73 - DNA, 16 bp, Artificial Sequence
  Description of Artificial Sequence 5′ PCR Primer with parsing bases TCGT
cgacggtatcggtcgt

SEQ ID NO: 74 - DNA, 16 bp, Artificial Sequence
  Description of Artificial Sequence 5′ PCR Primer with parsing bases TGGT
cgacggtatcggtggt

SEQ ID NO: 75 - DNA, 16 bp, Artificial Sequence
  Description of Artificial Sequence 5′ PCR Primer with parsing bases TGTT
cgacggtatcggtgtt

SEQ ID NO: 76 - DNA, 16 bp, Artificial Sequence
  Description of Artificial Sequence 5′ PCR Primer with parsing bases TTAC
cgacggtatcggttac

SEQ ID NO: 77 - DNA, 16 bp, Artificial Sequence
  Description of Artificial Sequence 5′ PCR Primer with parsing bases TTCC
cgacggtatcggttcc

SEQ ID NO: 78 - DNA, 16 bp, Artificial Sequence
  Description of Artificial Sequence 5′ PCR Primer with parsing bases TGGG
cgacggtatcggtggg

SEQ ID NO: 79 - DNA, 16 bp, Artificial Sequence
  Description of Artificial Sequence 5′ PCR Primer with parsing bases CCGG
cgacggtatcggccgg

SEQ ID NO: 80 - DNA, 16 bp, Artificial Sequence
  Description of Artificial Sequence 5′ PCR Primer with parsing bases CGGT
cgacggtatcggcggt

SEQ ID NO: 81 - DNA, 16 bp, Artificial Sequence
  Description of Artificial Sequence 5′ PCR Primer with parsing bases TGCA
cgacggtatcggtgca

SEQ ID NO: 82 - DNA, 16 bp, Artificial Sequence
  Description of Artificial Sequence 5′ PCR Primer with parsing bases CCGA
cgacggtatcggccga

SEQ ID NO: 83 - DNA, 16 bp, Artificial Sequence
  Description of Artificial Sequence 5′ PCR Primer with parsing bases CGGT
cgacggtatcggcggt

SEQ ID NO: 84 - DNA, 16 bp, Artificial Sequence
  Description of Artificial Sequence 5′ PCR Primer with parsing bases TTAG
cgacggtatcggttag

SEQ ID NO: 85 - DNA, 16 bp, Artificial Sequence
  Description of Artificial Sequence 5′ PCR Primer with parsing bases TTCA
cgacggtatcggttca

SEQ ID NO: 86 - DNA, 30 bp, Artificial Sequence
  Description of Artificial Sequence Extende d-TOGA primer for REC1_1 clone
gatcgaatccggaagccgcgcatcactgag

SEQ ID NO: 87 - DNA, 30 bp, Artificial Sequence
  Description of Artificial Sequence Extende d-TOGA primer for REC1_2 clone
gatcgaatccggaatcctgggagaggccaa

SEQ ID NO: 88 - DNA, 30 bp, Artificial Sequence
  Description of Artificial Sequence Extende d-TOGA primer for REC1_3 clone
gatcgaatccggacggggagggagcagaga

SEQ ID NO: 89 - DNA, 30 bp, Artificial Sequence
  Description of Artificial Sequence Extende d-TOGA primer for REC1_5 clone
gatcgaatccggagtgggaagaggcctggg

SEQ ID NO: 90 - DNA, 30 bp, Artificial Sequence
  Description of Artificial Sequence Extende d-TOGA primer for REC1_6 clone
gatcgaatccggatccagcggcgccgcgcg

SEQ ID NO: 91 - DNA, 30 bp, Artificial Sequence
  Description of Artificial Sequence Extende d-TOGA primer for REC1_7 clone
gatcgaatccggcacacgggcgcaagaaga

SEQ ID NO: 92 - DNA, 30 bp, Artificial Sequence
  Description of Artificial Sequence Extende d-TOGA primer for REC1_8 clone
gatcgaatccggcccgataggtgggtgccc

SEQ ID NO: 93 - DNA, 30 bp, Artificial Sequence
  Description of Artificial Sequence Extende d-TOGA primer for REC1_10 clone
gatcgaatccggctgagtgaaggaatgatg

SEQ ID NO: 94 - DNA, 30 bp, Artificial Sequence
  Description of Artificial Sequence Extende d-TOGA primer for REC1_11 clone
gatcgaatccgggcattaaacactctcaca

SEQ ID NO: 95 - DNA, 30 bp, Artificial Sequence
  Description of Artificial Sequence Extende d-TOGA primer for REC1_12 clone
gatcgaatccgggccctccattgtccaccg

SEQ ID NO: 96 - DNA, 30 bp, Artificial Sequence
  Description of Artificial Sequence Extende d-TOGA primer for REC1_13 clone
gatcgaatccgggccccctccatccccagt

SEQ ID NO: 97 - DNA, 30 bp, Artificial Sequence
  Description of Artificial Sequence Extende d-TOGA primer for REC1_15 clone
gatcgaatccggtcagcctcccccccaaaa

SEQ ID NO: 98 - DNA, 30 bp, Artificial Sequence
  Description of Artificial Sequence Extende d-TOGA primer for REC1_16 clone
gatcgaatccggtcggcggcgctccgcgta

SEQ ID NO: 99 - DNA, 30 bp, Artificial Sequence
  Description of Artificial Sequence Extende d-TOGA primer for REC1_17 clone
gatcgaatccggtcgtatcggcagtgactt

SEQ ID NO: 100 - DNA, 30 bp, Artificial Sequence
  Description of Artificial Sequence Extende d-TOGA primer for REC1_18 clone
gatcgaatccggtggtggccaggacggagc

SEQ ID NO: 101 - DNA, 30 bp, Artificial Sequence
  Description of Artificial Sequence Extende d-TOGA primer for REC1_19 clone
gatcgaatccggtgttaccagtgcgaaaag

SEQ ID NO: 102 - DNA, 30 bp, Artificial Sequence
  Description of Artificial Sequence Extende d-TOGA primer for REC1_20 clone
gatcgaatccggttaccagtgtcattctcc

SEQ ID NO: 103 - DNA, 30 bp, Artificial Sequence
  Description of Artificial Sequence Extende d-TOGA primer for REC1_21 clone
gatcgaatccggttcctcgctcgagccact

SEQ ID NO: 104 - DNA, 30 bp, Artificial Sequence
  Description of Artificial Sequence Extende d-TOGA primer for REC1_22 clone
gatcgaatccggtgggccttggcttctcca

SEQ ID NO: 105 - DNA, 30 bp, Artificial Sequence
  Description of Artificial Sequence Extende d-TOGA primer for REC1_24 clone
gatcgaatccggccgggccacgttggaagc

SEQ ID NO: 106 - DNA, 30 bp, Artificial Sequence
  Description of Artificial Sequence Extende d-TOGA primer for REC1_28 clone
gatcgaatccggcggtgataaaacaataaa

SEQ ID NO: 107 - DNA, 30 bp, Artificial Sequence
  Description of Artificial Sequence Extende d-TOGA primer for REC1_31 clone
gatcgaatccggtgcactagctttagtctc

SEQ ID NO: 108 - DNA, 30 bp, Artificial Sequence
  Description of Artificial Sequence Extende d-TOGA primer for REC1_33 clone
gatcgaatccggccgatgtctctctctctc

SEQ ID NO: 109 - DNA, 30 bp, Artificial Sequence
  Description of Artificial Sequence Extende d-TOGA primer for REC1_34 clone
gatcgaatccggcggtgcctggaggaccct

SEQ ID NO: 110 - DNA, 30 bp, Artificial Sequence
  Description of Artificial Sequence Extende d-TOGA primer for REC1_36 clone
gatcgaatccggttagcaaccctgaagtga

SEQ ID NO: 111 - DNA, 30 bp, Artificial Sequence
  Description of Artificial Sequence Extende d-TOGA primer for REC1_37 clone
gatcgaatccggttcacaagcacagggaga

SEQ ID NO: 112 - DNA, 30 bp, Artificial Sequence
  Description of Artificial Sequence Extende d-TOGA primer for clone with digital address ATAC 176
gatcgaatccggatacaaactgggcgtttg

SEQ ID NO: 113 - DNA, 30 bp, Artificial Sequence
  Description of Artificial Sequence Extende d-TOGA primer for clone with digital address CAGT 170
gatcgaatccggcagtggcccacctagcca

SEQ ID NO: 114 - DNA, 30 bp, Artificial Sequence
  Description of Artificial Sequence Extende d-TOGA primer for clone with digital address CCGA 259
gatcgaatccggccgaggactggaaccagg

SEQ ID NO: 115 - DNA, 30 bp, Artificial Sequence
  Description of Artificial Sequence Extende d-TOGA primer for clone with digital address GTCC 111
gatcgaatccgggtcccagggcagcccgtc

SEQ ID NO: 116 - DNA, 30 bp, Artificial Sequence
  Description of Artificial Sequence Extende d-TOGA primer for clone with digital address GTTG 116
gatcgaatccgggttgtgggctgtgacttc

SEQ ID NO: 117 - DNA, 30 bp, Artificial Sequence
  Description of Artificial Sequence Extende d-TOGA primer for clone with digital address TATT 315
gatcgaatccggtatttggatggaataaat

SEQ ID NO: 118 - DNA, 30 bp, Artificial Sequence
  Description of Artificial Sequence Extende d-TOGA primer for clone with digital address CCTG 172
gatcgaatccggcctggccctgagcaagta

SEQ ID NO: 119 - DNA, 16 bp, Artificial Sequence
  Description of Artificial Sequence 5′ PCR Primer with parsing bases ATAC
cgacggtatcggatac

SEQ ID NO: 120 - DNA, 16 bp, Artificial Sequence
  Description of Artificial Sequence 5′ PCR Primer with parsing bases CAGT
cgacggtatcggcagt

SEQ ID NO: 121 - DNA, 16 bp, Artificial Sequence
  Description of Artificial Sequence 5′ PCR Primer with parsing bases GTCC
cgacggtatcgggtcc

SEQ ID NO: 122 - DNA, 16 bp, Artificial Sequence
  Description of Artificial Sequence 5′ PCR Primer with parsing bases GTTG
cgacggtatcgggttg

SEQ ID NO: 123 - DNA, 16 bp, Artificial Sequence
  Description of Artificial Sequence 5′ PCR Primer with parsing bases TATT
cgacggtatcggtatt

SEQ ID NO: 124 - DNA, 16 bp, Artificial Sequence
  Description of Artificial Sequence REC1_7 internal PCR Primer
cgggcgcaagaagaca

SEQ ID NO: 125 - DNA, 21 bp, Artificial Sequence
  Description of Artificial Sequence REC1_7 internal PCR Primer
tgctagccccaaagataaaaa

SEQ ID NO: 126 - DNA, 30 bp, Artificial Sequence
  Description of Artificial Sequence Extended- TOGA primer for REC1_4 clone
gatcgaatccggagtatgatgctctggacg

SEQ ID NO: 127 - DNA, 30 bp, Artificial Sequence
  Description of Artificial Sequence Extended- TOGA primer for REC1_25 clone
gatcgaatccggtagctcaaaagaaagcaa

SEQ ID NO: 128 - DNA, 30 bp, Artificial Sequence
  Description of Artificial Sequence Extended- TOGA primer for REC1_29 clone
gatcgaatccggtaatatcatgtgcacatc

SEQ ID NO: 129 - DNA, 30 bp, Artificial Sequence
  Description of Artificial Sequence Extended- TOGA primer for REC1_32 clone
gatcgaatccgggatacgtacctggctcct

SEQ ID NO: 130 - DNA, 30 bp, Artificial Sequence
  Description of Artificial Sequence Extended- TOGA primer for REC1_35 clone
gatcgaatccggcttacaagaccagagtaa

SEQ ID NO: 131 - DNA, 30 bp, Artificial Sequence
  Description of Artificial Sequence Extended- TOGA primer for REC1_38 clone
gatcgaatccgggtgcgcattttatttgtg

SEQ ID NO: 132 - DNA, 30 bp, Artificial Sequence
  Description of Artificial Sequence Extended- TOGA primer for REC1_39 clone
gatcgaatccggctgggtgctggtgtcgcc

SEQ ID NO: 133 - DNA, 30 bp, Artificial Sequence
  Description of Artificial Sequence Extended- TOGA primer for REC1_40 clone
gatcgaatccggctgcagaagcaagaaccc

SEQ ID NO: 134 - DNA, 30 bp, Artificial Sequence
  Description of Artificial Sequence Extended- TOGA primer for REC1_42 clone
gatcgaatccggacacggacaggattgaca

SEQ ID NO: 135 - DNA, 30 bp, Artificial Sequence
  Description of Artificial Sequence Extended- TOGA primer for REC1_43 clone
gatcgaatccggaggcaggccgagaagaac

SEQ ID NO: 136 - DNA, 30 bp, Artificial Sequence
  Description of Artificial Sequence Extended- TOGA primer for REC1_44 clone
gatcgaatccggaggtcgacaggacccaga

SEQ ID NO: 137 - DNA, 30 bp, Artificial Sequence
  Description of Artificial Sequence Extended- TOGA primer for REC1_45 clone
gatcgaatccggcagtagcagccatttggg

SEQ ID NO: 138 - DNA, 30 bp, Artificial Sequence
  Description of Artificial Sequence Extended- TOGA primer for REC1_46 clone
gatcgaatccggcgcttgacggttgtcaca

SEQ ID NO: 139 - DNA, 30 bp, Artificial Sequence
  Description of Artificial Sequence Extended- TOGA primer for REC1_47 clone
gatcgaatccgggatcctgtcagaaggggg

SEQ ID NO: 140 - DNA, 30 bp, Artificial Sequence
  Description of Artificial Sequence Extended- TOGA primer for REC1_48 clone
gatcgaatccggttacatacaaggatcctg

SEQ ID NO: 141 - DNA, 30 bp, Artificial Sequence
  Description of Artificial Sequence Extended- TOGA primer for REC1_49 clone
gatcgaatccggtccgtggctcgcatcgcc

SEQ ID NO: 142 - DNA, 30 bp, Artificial Sequence
  Description of Artificial Sequence Extended- TOGA primer for REC1_50 clone
gatcgaatccggaggagcccaagtgggtag

SEQ ID NO: 143 - DNA, 30 bp, Artificial Sequence
  Description of Artificial Sequence Extended- TOGA primer for REC1_51 clone
gatcgaatccgggactgtgagaaataaacg

SEQ ID NO: 144 - DNA, 30 bp, Artificial Sequence
  Description of Artificial Sequence Extended- TOGA primer for REC1_52 clone
gatcgaatccggcgggcacaggcccaaagg

SEQ ID NO: 145 - DNA, 30 bp, Artificial Sequence
  Description of Artificial Sequence Extended- TOGA primer for REC1_53 clone
gatcgaatccggccccgccctgcttccacg

SEQ ID NO: 146 - DNA, 30 bp, Artificial Sequence
  Description of Artificial Sequence Extended- TOGA primer for REC1_54 clone
gatcgaatccggtgctacttgagacaaaag

SEQ ID NO: 147 - DNA, 30 bp, Artificial Sequence
  Description of Artificial Sequence Extended- TOGA primer for REC1_55 clone
gatcgaatccggatttccacctaccacttc

SEQ ID NO: 148 - DNA, 30 bp, Artificial Sequence
  Description of Artificial Sequence Extended- TOGA primer for REC1_56 clone
gatcgaatccggtgacattcctggcgtccg

SEQ ID NO: 149 - DNA, 30 bp, Artificial Sequence
  Description of Artificial Sequence Extended- TOGA primer for REC1_57 clone
gatcgaatccgggtgaccagacaggccatg

SEQ ID NO: 150 - DNA, 30 bp, Artificial Sequence
  Description of Artificial Sequence Extended- TOGA primer for REC1_58 clone
gatcgaatccggagttataccacagtgatt

SEQ ID NO: 151 - DNA, 30 bp, Artificial Sequence
  Description of Artificial Sequence Extended- TOGA primer for REC1_59 clone
gatcgaatccggattatcatgctctggacg

SEQ ID NO: 152 - DNA, 30 bp, Artificial Sequence
  Description of Artificial Sequence Extended- TOGA primer for REC1_60 clone
gatcgaatccgggcgttgtactgcgtttgg

SEQ ID NO: 153 - DNA, 30 bp, Artificial Sequence
  Description of Artificial Sequence Extended- TOGA primer for REC1_61 clone
gatcgaatccggaccacggtgccacattgc

SEQ ID NO: 154 - DNA, 30 bp, Artificial Sequence
  Description of Artificial Sequence Extended- TOGA primer for REC1_62 clone
gatcgaatccgggagaggtcctgactctct

SEQ ID NO: 155 - DNA, 24 bp, Artificial Sequence
  Description of Artificial Sequence RT-PCR 5′ PCR primer for REC1_1 clone
tccagggtcagaggtcgatgtgta

SEQ ID NO: 156 - DNA, 21 bp, Artificial Sequence
  Description of Artificial Sequence RT-PCR 3′ PCR primer for REC1_1 clone
gcagcaacagaggggagaggt

SEQ ID NO: 157 - DNA, 19 bp, Artificial Sequence
  Description of Artificial Sequence RT-PCR 5′ PCR primer for REC1_2 clone
aatcctgggagaggccaag

SEQ ID NO: 158 - DNA, 21 bp, Artificial Sequence
  Description of Artificial Sequence RT-PCR 3′ PCR primer for REC1_2 clone
ttgaagcaaaagcgctctctt

SEQ ID NO: 159 - DNA, 18 bp, Artificial Sequence
  Description of Artificial Sequence RT-PCR 5′ PCR primer for REC1_3 clone
gacggggagggagcagag

SEQ ID NO: 160 - DNA, 21 bp, Artificial Sequence
  Description of Artificial Sequence RT-PCR 3′ PCR primer for REC1_3 clone
tgctctccttggaggatcaca

SEQ ID NO: 161 - DNA, 16 bp, Artificial Sequence
  Description of Artificial Sequence RT-PCR 5′ PCR primer for REC1_5 clone
gaggcctgggctcaag

SEQ ID NO: 162 - DNA, 21 bp, Artificial Sequence
  Description of Artificial Sequence RT-PCR 3′ PCR primer for REC1_5 clone
gatgattaaaatgtccccagc

SEQ ID NO: 163 - DNA, 16 bp, Artificial Sequence
  Description of Artificial Sequence RT-PCR 5′ PCR primer for REC1_6 clone
cgctctcctgccagcc

SEQ ID NO: 164 - DNA, 21 bp, Artificial Sequence
  Description of Artificial Sequence RT-PCR 3′ PCR primer for REC1_6 clone
ttcgctgcagcaagaaacttt

SEQ ID NO: 165 - DNA, 16 bp, Artificial Sequence
  Description of Artificial Sequence RT-PCR 5′ PCR primer for REC1_7 clone
cgggcgcaagaagaca

SEQ ID NO: 166 - DNA, 21 bp, Artificial Sequence
  Description of Artificial Sequence RT-PCR 3′ PCR primer for REC1_7 clone
tgctagccccaaagataaaaa

SEQ ID NO: 167 - DNA, 16 bp, Artificial Sequence
  Description of Artificial Sequence RT-PCR 5′ PCR primer for REC1_8 clone
gtgggtgccctggctg

SEQ ID NO: 168 - DNA, 21 bp, Artificial Sequence
  Description of Artificial Sequence RT-PCR 3′ PCR primer for REC1_8 clone
ccactttctaccccaggctcc

SEQ ID NO: 169 - DNA, 21 bp, Artificial Sequence
  Description of Artificial Sequence RT-PCR 5′ PCR primer for REC1_10 clone
cacctgccctcagagttgaca

SEQ ID NO: 170 - DNA, 20 bp, Artificial Sequence
  Description of Artificial Sequence RT-PCR 3′ PCR primer for REC1_10 clone
ggcagacagggttcaccaca

SEQ ID NO: 171 - DNA, 17 bp, Artificial Sequence
  Description of Artificial Sequence RT-PCR 5′ PCR primer for REC1_12 clone
caccgcaaatgcttcta

SEQ ID NO: 172 - DNA, 21 bp, Artificial Sequence
  Description of Artificial Sequence RT-PCR 3′ PCR primer for REC1_12 clone
tttgtggtagcatatttcaga

SEQ ID NO: 173 - DNA, 19 bp, Artificial Sequence
  Description of Artificial Sequence RT-PCR 5′ PCR primer for REC1_13 clone
ccccctccatccccagtga

SEQ ID NO: 174 - DNA, 21 bp, Artificial Sequence
  Description of Artificial Sequence RT-PCR 3′ PCR primer for REC1_13 clone
cctcacacgtgggtggctcac

SEQ ID NO: 175 - DNA, 21 bp, Artificial Sequence
  Description of Artificial Sequence RT-PCR 5′ PCR primer for REC1_16 clone
cgtagactcgccgaggttttc

SEQ ID NO: 176 - DNA, 21 bp, Artificial Sequence
  Description of Artificial Sequence RT-PCR 3′ PCR primer for REC1_16 clone
tttttggcgtctcacacttca

SEQ ID NO: 177 - DNA, 20 bp, Artificial Sequence
  Description of Artificial Sequence RT-PCR 5′ PCR primer for REC1_17 clone
ggtcgtatcggcagtgactt

SEQ ID NO: 178 - DNA, 21 bp, Artificial Sequence
  Description of Artificial Sequence RT-PCR 3′ PCR primer for REC1_17 clone
ctgggaaaataagcctcattg

SEQ ID NO: 179 - DNA, 17 bp, Artificial Sequence
  Description of Artificial Sequence RT-PCR 5′ PCR primer for REC1_18 clone
acggagccgctcaggga

SEQ ID NO: 180 - DNA, 21 bp, Artificial Sequence
  Description of Artificial Sequence RT-PCR 3′ PCR primer for REC1_18 clone
ccctcggcaccagagtaggaa

SEQ ID NO: 181 - DNA, 21 bp, Artificial Sequence
  Description of Artificial Sequence RT-PCR 5′ PCR primer for REC1_19 clone
ccggtgttaccagtgcgaaaa

SEQ ID NO: 182 - DNA, 21 bp, Artificial Sequence
  Description of Artificial Sequence RT-PCR 3′ PCR primer for REC1_19 clone
ggtctgccccatccttgtcag

SEQ ID NO: 183 - DNA, 21 bp, Artificial Sequence
  Description of Artificial Sequence RT-PCR 5′ PCR primer for REC1_20 clone
cggttaccagtgtcattctcc

SEQ ID NO: 184 - DNA, 21 bp, Artificial Sequence
  Description of Artificial Sequence RT-PCR 3′ PCR primer for REC1_20 clone
ggggtttaggccaaatgttta

SEQ ID NO: 185 - DNA, 16 bp, Artificial Sequence
  Description of Artificial Sequence RT-PCR 5′ PCR primer for REC1_21 clone
cctcgctcgagccact

SEQ ID NO: 186 - DNA, 21 bp, Artificial Sequence
  Description of Artificial Sequence RT-PCR 3′ PCR primer for REC1_21 clone
tctatcacagcgagacacaga

SEQ ID NO: 187 - DNA, 16 bp, Artificial Sequence
  Description of Artificial Sequence RT-PCR 5′ PCR primer for REC1_22 clone
gggccttggcttctcc

SEQ ID NO: 188 - DNA, 21 bp, Artificial Sequence
  Description of Artificial Sequence RT-PCR 3′ PCR primer for REC1_22 clone
atgcaggtcagaaagaattgg

SEQ ID NO: 189 - DNA, 20 bp, Artificial Sequence
  Description of Artificial Sequence RT-PCR 5′ PCR primer for REC1_24 clone
cgaacactggaatgcatata

SEQ ID NO: 190 - DNA, 21 bp, Artificial Sequence
  Description of Artificial Sequence RT-PCR 3′ PCR primer for REC1_24 clone
aaccctttattacaagtcacg

SEQ ID NO: 191 - DNA, 16 bp, Artificial Sequence
  Description of Artificial Sequence RT-PCR 5′ PCR primer for REC1_36 clone
gcaaccctgaagtgat

SEQ ID NO: 192 - DNA, 21 bp, Artificial Sequence
  Description of Artificial Sequence RT-PCR 3′ PCR primer for REC1_36 clone
aattctcattcagtaaagatt

SEQ ID NO: 193 - DNA, 59 bp, Artificial Sequence
  Description of Artificial Sequence 5′ adapt er primer for directsequencing
tcccagtcacgacgttgtaaaacgacggctcatatgaattaggtgaccgacggtatcgg

SEQ ID NO: 194 - DNA, 46 bp, Artificial Sequence
  Description of Artificial Sequence 3′ adapt er primer for directsequencing
cagcggataacaatttcacacagggagctccaccgcggtggcggcc

SEQ ID NO: 195 - DNA, 23 bp, Artificial Sequence
  Description of Artificial Sequence 5′ sequen cing primer for direct sequencing
cccagtcacgacgttgtaaaacg

SEQ ID NO: 196 - DNA, 19 bp, Artificial Sequence
  Description of Artificial Sequence 3′ sequencing primer for direct sequencing
ttttttttttttttttttv

SEQ ID NO: 197 - DNA, 35 bp, Artificial Sequence
  Description of Artificial Sequence 3′ sequencing primer for direct sequencing
ggtggcggccgcaggaatttttttttttttttttt

(19) United States
(12) Patent Application Publication
(10) Pub. No.: US 2003/0215817 A1
(43) Pub. Date: Nov. 20, 2003

(54) Modulation of gene expression in formation of fatty atherosclerotic lesions

(76) Inventors: Amedeo Leonardi, Milano (IT); Abraham Sartani, Arese (IT); James R. Glass, Cardiff, CA (US); J. Gregor Sutcliffe, Cardiff-by-the-Sea, CA (US); Karl W. Hasel, Solana Beach, CA (US)

Correspondence Address: Brian M Berliner, O'Melveny & Myers, 400 South Hope Street, Los Angeles, CA, 90071-2899, US

(21) Appl. No.: 10/182,230

(22) PCT Filed: Jan. 25, 2001
(86) PCT No.: PCT/US01/02439

(51) Int. Cl. C12Q 1/68; C07H 21/04; C12N 9/00; C12P 21/02; C12N 5/06; C07K 14/47; C07K 16/40

(52) U.S. Cl. 435/6; 435/69.1; 435/183; 435/320.1; 435/325; 530/350; 530/388.26; 536/23.2

(57) ABSTRACT

Polynucleotides, polypeptides, kits and methods are provided related to genes regulated by the formation of fatty atherosclerotic lesions, and by administration of a dihydropyridine calcium antagonist, lercanidipine.

### REFERENCE TO RELATED APPLICATION

This application claims the benefit of U.S. Provisional Application S. No. 60/177,963, filed Jan. 25, 2000, which is incorporated herein by reference in its entirety.

### BACKGROUND OF THE INVENTION

Atherosclerosis is a disease-state in which the walls of the arteries become thickened and lose their elasticity, resulting in a decreased ability to pump blood to peripheral organs and withstand systemic pressure. The most common form of atherosclerosis is caused by the build-up of fatty deposits in the innermost layer of the artery wall. Another form of atherosclerosis, Monckeberg's atherosclerosis, results from the build-up of calcium deposits that destroys the middle layer of the artery wall. Yet another form of the disease involves the destruction of the smaller arteries, or arterioles. All three forms of the disease contribute to a decreased blood flow to vital organs, which can lead to a stroke, heart attack, or kidney failure. The current identified risk factors for developing atherosclerosis include high blood pressure, increased plasma levels of low density lipoprotein, decreased plasma levels of high density lipoprotein, diabetes, obesity, and a genetic predisposition.

Increasing knowledge of the pathogenesis of atherosclerosis suggests that prevention of cardiovascular disease will involve not only the correction of the above risk factors, but also the direct pharmacological control of atherogenic processes occurring in the arterial wall (Ross, R., Nature, 362:801-809 (1993)). The arterial wall is made up of three distinct layers: the intima, media, and adventitia. The innermost layer, the intima, consists primarily of smooth muscle cells and a thin layer of endothelial cells which lines the lumen of the artery. The media is a region of elastic and collagen fibers containing, inter alia, smooth muscle cells, fibroblasts, and macrophages. The collagenous adventitia layer comprises the outer component of the arterial wall.

Atherosclerotic lesions are characterized by numerous biochemical changes, including an increased number of cells, mainly macrophages and smooth muscle cells, in the intima and the appearance of macrophages filled with lipid droplets (foam cells). The presence of lipid deposits and other changes in the composition of the matrix disrupt the intimal architecture, resulting in intimal disorganization and the thickening and deformation of the arterial wall. It is generally believed that atherosclerotic lesions result from specific cell reactions initiated by the accumulation of atherogenic, plasma-derived lipoproteins in the arterial intima. Lesion size and complexity increase as lipoprotein accumulation in the intima continues and increases. For example, increased levels of apolipoprotein B have been associated with a thickened intima in the aorta of swine (Hoff et al., Lab. Invest., 48:492-504 (1983)) and human distal abdominal aortas (Spring et al., Exp. Mol. Pathol., 51:179-185 (1989)). In addition, studies have shown that rabbits fed a high cholesterol diet for 8 to 16 days have increased low-density lipoprotein levels in lesion-prone regions of the aortic intima before the appearance of macrophage foam cells (Schwenke et al., Arteriosclerosis, 9:895-907 (1989); Schwenke et al., Arteriosclerosis, 9:908-918 (1989)). Further, immunohistochemical studies have shown increased levels of apolipoproteins A and B in regions of human arteries with intimal thickening before the appearance of foam cells (Vollmer et al., Virchows Arch. Pathol. Anat. Histopath., 419:79-88 (1991)).

Lipoproteins may be trapped in the intima by matrix components and then undergo modification, including, for example, oxidation (Schwenke et al., Arteriosclerosis, 9:895-907 (1989); Schwenke et al., Arteriosclerosis, 9:908-918 (1989); Fry, D. L., Arteriosclerosis, 7:88-100 (1987); Steinberg et al., JAMA, 264:3047-3052 (1990)). The modified lipoproteins maybe internalized by macrophages and/or smooth muscle cells through native lipoprotein receptors or via a scavenger receptor pathway (Pitas, R. E., J. Biol. Chem., 265:12722-12727 (1989)). Although endocytosed lipids are generally broken down and re-esterified, there is some evidence that cells retain oxidized lipids in non-degraded or minimally degraded forms (Sparrow et al., J. Biol. Chem., 264:2599-2604 (1989)). The presence of oxidized lipoproteins may, in turn, result in the increased presence of monocytes in the intima. Although, isolated macrophages are normally present in the intima (Stary, H. C., Atherosclerosis, 64:91-108 (1987); Stary, H. C., Eur. Heart J., 11:E3-19 (1990)), in vivo studies revealed an elevated level of intimal macrophages under conditions of hypercholesterolemia, resulting from an increased movement of plasma monocytes into the intima (Spraragen et al., Circ., 40:1-24 (1969); Gerrity, R. G., Am. J. Pathol., 103:191-200 (1981); Gerrity, R. G., Am. J. Pathol., 103:181-190 (1981); Lewis et al., Ann. N.Y. Acad. Sci., 454:91-100 (1985)). This movement may be a response to the increased presence of oxidized lipoproteins, which have been shown to be chemotactic for monocytes in vitro (Quinn et al., Proc. Nat'l. Acad. Sci., 84:2995-2998 (1987)).

Others have hypothesized that atherosclerotic lesions are initiated in response to cell injury, in particular, injury resulting from denudation of the endothelial cell layer (Ross et al., New Eng. J. Med., 295:420-425 (1976); Velican et al., Atherosclerosis, 37:33-46 (1980); Bondjers et al., Circ., 84:2-16 (1991)). Such injury results in smooth muscle cell migration from the media into the intima and proliferation within the intima, causing intimal thickening. Injured or activated endothelial cells may also produce leukocyte adherence molecules and secrete cytokines, which are chemotactic for leukocytes and smooth muscles cells, as well as produce growth factors for all of the cell types on or within the arterial wall (Gajdusek et al., J. Cell Biol., 85:467-472 (1980)). These processes trigger a cascade of events leading to morphological changes in the vessel wall and the development of vascular diseases (Ross (1993), supra; Jackson, et al., Hypertension, 20:713-736 (1992); Popma et al., Circ., 84:1426-1436 (1991)).

According to yet another hypothesis, platelet and/or fibrin deposits on the intima could initiate the development of atherosclerotic lesions. Several researchers have reported the microscopic observation of thin layers of fibrin and/or aggregated platelets on the endothelial surface of the intima (More et al., Arch. Pathol., 63:612-620 (1957); McMillan, G. C., Acta Cardiol., 11:43-62 (1965); Geer et al., Monogr. Atheroscler., 2:1-140 (1972); Spurlock et al., Scanning Microsc., 1:1359-1365 (1987)). However, it is not known whether such deposits can enlarge to become lesions in the absence of risk factors that favor lipid deposition.

Knowledge of the pathogenesis of atherosclerosis has prompted investigations into the possibility of direct pharmacological control of the pathological processes occurring in the arterial wall. Recent studies have focused on evaluating the direct effect of drug therapy on the cellular components of the arterial wall (Jackson et al., Hypertension, 20:713-736 (1992)). The anticipation is that by altering early events of the atherosclerotic process, the chances of halting or slowing the progression of the disease may be improved. Among the drugs under current investigation as anti-atherogenic agents are calcium channel blockers, or calcium antagonists, which are well-established in the treatment of a number of cardiovascular disorders (Nayler, W. G., Drugs, 46:40-47 (1993); Waters et al., Am. Heart J., 128:1309-1316 (1994)). There are three subclasses of calcium channel antagonists: the phenylalkylamine derivatives (e.g. verapamil), the benzothiazepines (e.g. diltiazem), and the diydropyridines (e.g. nifedipine, lercanidipine). All three subclasses modify calcium entry into cells by interacting with specific binding sites on the α₁ subunit of the L-type voltage-dependent calcium channel (Nayler (1993), supra).

Calcium antagonists have been studied extensively in both in vitro and in vivo experimental models (Bemini et al., Am. J. Cardiol., 64:1291-1341 (1989); Lichtor et al., Appl. Pathology, 7:8-18 (1989); Jackson et al., Hypertension, 20:713-736 (1992); Henry, P. D., Cardiovasc. Pharm., 16:512-515 (1990); Catapano, A., Eur. Heart J., 18:A80-A86 (1997)). In addition to evidence that calcium antagonists reduce blood pressure, experimental and clinical data indicate that calcium antagonists may protect against structural changes occurring in the vessel wall during the progression of atherosclerosis (Jackson (1992), supra; Nayler, W. G., Biochem. Pharmacol., 43:39-46 (1992); Lichtlen et al., Cardiovasc. Drugs Ther., 1:71-79 (1987); Parmley, W. W., Am. J. Med., 82:3-8 (1987)). Notably, several calcium-dependent processes contribute to atherogenesis, including lipid infiltration and oxidation, endothelial cell injury, chemotactic and growth factor activities, and smooth muscle cell migration and proliferation (Nayler (1993), supra; Catapano (1997), supra).

Further, in vivo studies have shown that calcium antagonists protect against lesions induced by cholesterol feeding, endothelial injury, and experimental calcinosis (Bemini et. al. (1989), supra; Keogh et al., J. Cardiovasc. Pharmacol., 16:528-525 (1990); Weinstein et al., Am J. Med., 86:27-32 (1989); Catapano, et. al., Ann. N.Y. Acad. Sci., 522:519-521 (1988)). In addition, calcium antagonists have been shown to decrease the accumulation of collagen, elastin, and proteoglycans in the arterial wall, following administration of compounds that induce atherosclerosis (Walters et al., J. Am. Coll. Cardiol., 15:116A (1990)).

The “anti-atherosclerotic” effects of calcium antagonists have been supported by several in vitro models. For instance, several calcium antagonists have been shown to inhibit the migration and proliferation of smooth muscle cells in vitro (Nomoto et al., Atheriosclerosis, 72:213-219 (1988); Jackson (1992), supra; Corsini et al., Pharmacol. Res., 27:299-307 (1993); Corsini et al., J. Vasc. Med. Biol., 5:111-119 (1994)). In addition, calcium antagonists have been reported to modulate LDL cholesterol metabolism (Bernini et al., Ann. N.Y. Acad. Sci., 522:390-398 (1988)) and to reduce fatty lesion development by interfering with cholesterol-esterification (Bernini et al., J. Hyperten., 11:S61-S66 (1993)). Also, several studies have shown that calcium antagonists inhibit the uptake of lipids by macrophages (Daugherty et al., Br. J. Pharm., 91:113-118 (1987); Bernini et al., J. Cardiovasc. Pharm., 18:542-545 (1991); Schmitz et al., Arteriosclerosis, 8:46-56 (1988); Stein et al., Arteriosclerosis, 7:578-584 (1987)).

A new dihydropyridine calcium antagonist, lercanidipine, has been shown to effectively reduce smooth muscle cell migration and proliferation in vitro (Corsini et al., J. Cardiovasc. Pharm., 28:687-694 (1996)). Lercanidipine has a high specificity for vascular smooth muscle cells and has a long duration of action due to its liposolubility. In addition, lercanidipine has been shown to modulate cholesterol acyl transferase activity and to act as an antioxidant for LDL in endothelial cell-mediated oxidation (Soma et al., Br. J. Pharm., 125:1471-1476 (1998)). Further in vivo studies have revealed that lercanidipine can inhibit both aortic fatty lesion deposition and carotid intimal hyperplasia (Soma (1998), supra).

Whether the effects of calcium antagonists on experimental atherosclerosis are linked to the blocking action on L-type channels remains unclear. Interestingly, lercanidipine presents with a chiral center that produces two enantiomers, of which the (R)-enantiomer is approximately 2-3 orders of magnitude less effective as a ligand to the calcium channel and in lowering blood pressure. Thus, studying the effects of the different enantiomers of lercanidipine provides a useful model for evaluating whether calcium antagonism plays a role in the anti-atherosclerotic activity of 1,4-dihydropyridine calcium antagonists.

In addition to the cited biochemical changes, hyperlipidemia-induced atherosclerosis is also associated with altered gene expression that initiates cell proliferation and de-differentiation in the intima of the arterial wall. The differential expression of genes following atherogenic stimulus has been described in several cells, including endothelial cells (de Waard et al., Gene, 226:1-8 (1999); De Graba T. J., Neurology, 49:S15-S 19 (1997)), smooth muscle cells (Sobue et al., Mol. Cell. Biochem., 190:105-118 (1999)) and macrophages (Chiu, D. S., Arterioscler. Thromb. Vasc. Biol., 17:2350-2358 (1997); Krettek et al., Arterioscler. Thromb. Vasc. Biol., 17:2395-2404 (1997)). One report has further demonstrated that lowering the dietary intake of lipid following atheriosclerotic plaque induction results in a reversion of differentiation-associated gene expression to that seen in the normal arterial wall (Aikawa et al., Circ. Res., 83:1015-1026 (1998). These data suggest the impact that gene expression changes have upon the development of the atherosclerotic phenotype.

Although the above studies have examined the differential expression of genes during early activation of arterial endothelial cells and have examined the expression of a few individual genes involved in the atherosclerotic phenotype, there has been no comprehensive study of the alteration of gene expression over time during the development of atherosclerotic lesions in aorta. Nor has there been a study of the effects of calcium antagonists in gene expression during atherosclerotic lesion development. Thus, the number and identity of the genes that are differentially expressed during atherosclerotic lesion development remains unknown. Further, the identity of those genes whose expression is affected by treatment with a calcium antagonist, such as lercanidipine, remains unknown.

The identification of genes whose level of expression is altered during the onset of atherosclerosis would not only contribute to the understanding of the disease pathology, but would also identify genes useful as diagnostic markers to indicate patients at risk for stroke or cardiovascular disease. Furthermore, the identification of differentially regulated genes would be useful to target genes for potential therapeutic intervention. In addition, the identification of genes whose expression is affected by calcium antagonists would advance the development of anti-atherosclerotic therapy that would target the specific action of calcium antagonists. The identification of such genes would also reveal key pathways that could be targeted for further investigation.

### SUMMARY OF THE INVENTION

The PCR-based Total Gene Expression (TOGA™) differential display system has been used to identify genes modulated during the development of atherosclerosis using an in vivo model wherein a fatty-streak lesion was induced in rabbit aorta. In addition, the TOGA system was used to study the effect of calcium antagonists, such as lercanidipine, on gene expression during fatty lesion development. Such studies are useful to determine the genes associated with the atherosclerotic phenotype and also those genes whose expression is affected by calcium antagonists. Such information can be used to identify proteins and genes that are useful in therapeutic and diagnostic applications in the treatment of atherosclerosis.

The present invention provides novel polynucleotides and the encoded polypeptides. Moreover, the present invention relates to vectors, host cells, antibodies, and recombinant methods for producing the polynucleotides and the polypeptides. One embodiment of the invention provides an isolated nucleic acid molecule comprising a polynucleotide chosen from the group consisting of

SEQ ID NO:1, SEQ ID NO:2, SEQ ID NO:3, SEQ ID NO:4, SEQ ID NO:5, SEQ ID NO:6,
SEQ ID NO:7, SEQ ID NO:8, SEQ ID NO:9, SEQ ID NO:10, SEQ ID NO:11, SEQ ID NO:12,
SEQ ID NO:13, SEQ ID NO:14, SEQ ID NO:15, SEQ ID NO:16, SEQ ID NO:17, SEQ ID NO:18,
SEQ ID NO:19, SEQ ID NO:20, SEQ ID NO:21, SEQ ID NO:22, SEQ ID NO:23, SEQ ID NO:24,
SEQ ID NO:25, SEQ ID NO:26, SEQ ID NO:27, SEQ ID NO:28, SEQ ID NO:29, SEQ ID NO:30,
SEQ ID NO:31, SEQ ID NO:32, SEQ ID NO:33, SEQ ID NO:34, SEQ ID NO:35, SEQ ID NO:36,
SEQ ID NO:37, SEQ ID NO:38, SEQ ID NO:39, SEQ ID NO:40, SEQ ID NO:41, SEQ ID NO:42,
SEQ ID NO:43, SEQ ID NO:44, SEQ ID NO:45, SEQ ID NO:46, SEQ ID NO:47, SEQ ID NO:48,
SEQ ID NO:49, SEQ ID NO:50, SEQ ID NO:51, SEQ ID NO:52, SEQ ID NO:53, SEQ ID NO:54
and SEQ ID NO:55.

Also provided is an isolated nucleic acid molecule comprising a polynucleotide at least 95% identical to any one of these isolated nucleic acid molecules and an isolated nucleic acid molecule at least ten bases in length that is hybridizable to any one of these isolated nucleic acid molecules under stringent conditions. Any one of these isolated nucleic acid molecules can comprise sequential nucleotide deletions from either the 5′-terminus or the 3′-terminus. Further provided is a recombinant vector comprising any one of these isolated nucleic acid molecules and a recombinant host cell comprising any one of these isolated nucleic acid molecules. Also provided is the gene corresponding to the cDNA sequence of any one of these isolated nucleic acids.

Another embodiment of the invention provides an isolated polypeptide encoded by a polynucleotide chosen from the group consisting of SEQ ID NO:1, SEQ ID NO:2, SEQ ID

SEQ ID NO:1, SEQ ID NO:2, SEQ ID NO:3, SEQ ID NO:4, SEQ ID NO:5, SEQ ID NO:6,
SEQ ID NO:7, SEQ ID NO:8, SEQ ID NO:9, SEQ ID NO:10, SEQ ID NO:11, SEQ ID NO:12,
SEQ ID NO:13, SEQ ID NO:14, SEQ ID NO:15, SEQ ID NO:16, SEQ ID NO:17, SEQ ID NO:18,
SEQ ID NO:19, SEQ ID NO:20, SEQ ID NO:21, SEQ ID NO:22, SEQ ID NO:23, SEQ ID NO:24,
SEQ ID NO:25, SEQ ID NO:26, SEQ ID NO:27, SEQ ID NO:28, SEQ ID NO:29, SEQ ID NO:30,
SEQ ID NO:31, SEQ ID NO:32, SEQ ID NO:33, SEQ ID NO:34, SEQ ID NO:35, SEQ ID NO:36,
SEQ ID NO:37, SEQ ID NO:38, SEQ ID NO:39, SEQ ID NO:40, SEQ ID NO:41, SEQ ID NO:42,
SEQ ID NO:43, SEQ ID NO:44, SEQ ID NO:45, SEQ ID NO:46, SEQ ID NO:47, SEQ ID NO:48,
SEQ ID NO:49, SEQ ID NO:50, SEQ ID NO:51, SEQ ID NO:52, SEQ ID NO:53, SEQ ID NO:54
and SEQ ID NO:55.

Also provided is an isolated nucleic acid molecule encoding any of these polypeptides, an isolated nucleic acid molecule encoding a fragment of any of these polypeptides, an isolated nucleic acid molecule encoding a polypeptide epitope of any of these polypeptides, and an isolated nucleic acid encoding a species homologue of any of these polypeptides. Preferably, any one of these polypeptides has biological activity. Optionally, any one of the isolated polypeptides comprises sequential amino acid deletions from either the C-terminus or the N-terminus. Further provided is a recombinant host cell that expresses any one of these isolated polypeptides.

Yet another embodiment of the invention comprises an isolated antibody that binds specifically to an isolated polypeptide encoded by a polynucleotide chosen from the group consisting of

SEQ ID NO:1, SEQ ID NO:2, SEQ ID NO:3, SEQ ID NO:4, SEQ ID NO:5, SEQ ID NO:6,
SEQ ID NO:7, SEQ ID NO:8, SEQ ID NO:9, SEQ ID NO:10, SEQ ID NO:11, SEQ ID NO:12,
SEQ ID NO:13, SEQ ID NO:14, SEQ ID NO:15, SEQ ID NO:16, SEQ ID NO:17, SEQ ID NO:18,
SEQ ID NO:19, SEQ ID NO:20, SEQ ID NO:21, SEQ ID NO:22, SEQ ID NO:23, SEQ ID NO:24,
SEQ ID NO:25, SEQ ID NO:26, SEQ ID NO:27, SEQ ID NO:28, SEQ ID NO:29, SEQ ID NO:30,
SEQ ID NO:31, SEQ ID NO:32, SEQ ID NO:33, SEQ ID NO:34, SEQ ID NO:35, SEQ ID NO:36,
SEQ ID NO:37, SEQ ID NO:3S, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54 and SEQ ID NO:55.

isolated antibody can be a monoclonal antibody or a polyclonal antibody.

Another embodiment of the invention provides a method for preventing, treating, modulating, or ameliorating a medical condition, such as atherosclerosis comprising administering to a mammalian subject a therapeutically effective amount of a polypeptide of the invention or a polynucleotide of the invention.

A further embodiment of the invention provides an isolated antibody that binds specifically to the isolated polypeptide of the invention. A preferred embodiment of the invention provides a method for preventing, treating, modulating, or ameliorating a medical condition, such as atherosclerosis, comprising administering to a mammalian subject a therapeutically effective amount of the antibody.

An additional embodiment of the invention provides a method of diagnosing a pathological condition or a susceptibility to a pathological condition in a subject. The method comprises determining the presence or absence of a mutation in a polynucleotide of the invention. A pathological condition or a susceptibility to a pathological condition, such as atherosclerosis is diagnosed based on the presence or absence of the mutation.

Even another embodiment of the invention provides a method of diagnosing a pathological condition or a susceptibility to a pathological condition, such as atherosclerosis in a subject. The method comprises detecting an alteration in expression of a polypeptide encoded by the polynucleotide of the invention, wherein the presence of an alteration in expression of the polypeptide is indicative of the pathological condition or susceptibility to the pathological condition. The alteration in expression can be an increase in the amount of expression or a decrease in the amount of expression. In a preferred embodiment a first biological sample is obtained from a patient suspected of having atherosclerosis and a second sample from a suitable comparable control source is obtained. The amount of at least one polypeptide encoded by a polynucleotide of the invention is determined in the first and second sample. The amount of the polypeptide in the first and second samples is determined. A patient is diagnosed as having atherosclerosis if the amount of the polypeptide in the first sample is greater than or less than the amount of the polypeptide in the second sample.

Another embodiment of the invention provides a method for identifying a binding partner to a polypeptide of the invention. A polypeptide of the invention is contacted with a binding partner and it is determined whether the binding partner effects an activity of the polypeptide.

Yet another embodiment of the invention is a method of identifying an activity of an expressed polypeptide in a biological assay. A polypeptide of the invention is expressed in a cell and isolated. The expressed polypeptide is tested for an activity in a biological assay and the activity of the expressed polypeptide is identified based on the test results.

Still another embodiment of the invention provides a substantially pure isolated DNA molecule suitable for use as a probe for genes regulated in atherosclerosis, chosen from the group consisting of the DNA molecules shown in

SEQ ID NO:1, SEQ ID NO:2, SEQ ID NO:3, SEQ ID NO:4, SEQ ID NO:5, SEQ ID NO:6,
SEQ ID NO:7, SEQ ID NO:8, SEQ ID NO:9, SEQ ID NO:10, SEQ ID NO:11, SEQ ID NO:12,
SEQ ID NO:13, SEQ ID NO:14, SEQ ID NO:15, SEQ ID NO:16, SEQ ID NO:17, SEQ ID NO:18,
SEQ ID NO:19, SEQ ID NO:20, SEQ ID NO:21, SEQ ID NO:22, SEQ ID NO:23, SEQ ID NO:24,
SEQ ID NO:25, SEQ ID NO:26, SEQ ID NO:27, SEQ ID NO:28, SEQ ID NO:29, SEQ ID NO:30,
SEQ ID NO:31, SEQ ID NO:32, SEQ ID NO:33, SEQ ID NO:34, SEQ ID NO:35, SEQ ID NO:36,
SEQ ID NO:37, SEQ ID NO:38, SEQ ID NO:39, SEQ ID NO:40, SEQ ID NO:41, SEQ ID NO:42,
SEQ ID NO:43, SEQ ID NO:44, SEQ ID NO:45, SEQ ID NO:46, SEQ ID NO:47, SEQ ID NO:48,
SEQ ID NO:49, SEQ ID NO:50, SEQ ID NO:51, SEQ ID NO:52, SEQ ID NO:53, SEQ ID NO:54
and SEQ ID NO:55.

Even another embodiment of the invention provides a kit for detecting the presence of a polypeptide of the invention in a mammalian tissue sample. The kit comprises a first antibody which immunoreacts with a mammalian protein encoded by a gene corresponding to the polynucleotide of the invention or with a polypeptide encoded by the polynucleotide in an amount sufficient for at least one assay and suitable packaging material. The kit can further comprise a second antibody that binds to the first antibody. The second antibody can be labeled with enzymes, radioisotopes, fluorescent compounds, colloidal metals, chemiluminescent compounds, phosphorescent compounds, or bioluminescent compounds.

Another embodiment of the invention provides a kit for detecting the presence of genes encoding a protein comprising a polynucleotide of the invention, or fragment thereof having at least 10 contiguous bases, in an amount sufficient for at least one assay, and suitable packaging material.

Yet another embodiment of the invention provides a method for detecting the presence of a nucleic acid encoding a protein in a mammalian tissue sample. A polynucleotide of the invention or fragment thereof having at least 10 contiguous bases is hybridized with the nucleic acid of the sample. The presence of the hybridization product is detected.

### BRIEF DESCRIPTION OF THE DRAWINGS

These and other features, aspects, and advantages of the present invention will become better understood with reference to the following description, appended claims, and accompanying drawings where:

FIGS. 1A-G is a graphical representation of the results of TOGA analysis using a 5′ PCR primer with parsing bases AAGC, showing PCR products produced from mRNA extracted from (A) control aorta at day 0 (no cholesterol, no lercanidipine), (B) lercanidipine-treated aorta at day 0 (no cholesterol, 3 mg/kg/week lercanidipine for 2 weeks), (C) control aorta at day 14 of cholesterol administration (1.6 g/day cholesterol, no lercanidipine), (D) lercanidipine-treated aorta at day 14 of cholesterol administration (1.6 g/day cholesterol, 3 mg/kg/week lercanidipine for 4 weeks), (E) R-lercanidipine treated aorta at day 14 of cholesterol administration (1.6 g/day cholesterol, 3 mg/kg/week R-lercanidipine for 4 weeks), (F) control aorta at week 8 of cholesterol administration (1.6 g cholesterol, no lercanidipine), (G) lercanidipine treated aorta at week 8 of cholesterol administration (1.6 g/day cholesterol, 3 mg/kg/week lercanidipine for 10 weeks), where the vertical index line indicates a PCR product of about 288 b.p. that is up-regulated during fatty lesions development in rabbit aorta;

FIGS. 2A-C is a graphical representation of more detailed analysis of the 288 b.p. PCR product indicated in FIG. 1, using the extended TOGA primer G-A-T-C-G-A-A-T-C-C-G-G-A-A-G-C-C-G-C-G-C-A-T-C-A-C-T-G-A-G (SEQ ID NO: 86);

FIGS. 3A-G is a graphical representation of the results of TOGA analysis using a 5′ PCR primer with parsing bases CACA, showing PCR products produced from mRNA extracted from (A) control aorta at day 0 (no cholesterol, no lercanidipine), (B) lercanidipine-treated aorta at day 0 (no cholesterol, 3 mg/kg/week lercanidipine for 2 weeks), (C) control aorta, at day 14 of cholesterol administration (1.6 g/day cholesterol, no lercanidipine), (D) lercanidipine-treated aorta at day 14 of cholesterol administration (1.6 g/day cholesterol, 3 mg/kg/week lercanidipine for 4 weeks), (E) R-lercanidipine treated aorta of day 14 of cholesterol administration (1.6 g/day cholesterol, 3 mg/kg/week R-lercanidipine for 4 weeks), (F) control aorta of week 8 of cholesterol administration (1.6 g cholesterol, no lercanidipine), (G) lercanidipine-treated aorta of week 8 of cholesterol administration (1.6 g/day cholesterol, 3 mg/kg/week lercanidipine for 10 weeks), where the vertical index line indicates a PCR product of about 282 b.p. that is down-regulated during fatty lesion development in rabbit aorta; and

FIGS. 4A-C is a graphical representation of more detailed analysis of the 282 b.p. PCR product indicated in FIG. 3, using the extended TOGA primer G-A-T-C-G-A-A-T-C-C-G-G-C-A-C-A-C-G-G-G-C-G-C-A-A-G-A-A-G-A (SEQ ID NO: 91).

FIGS. 5A-C is a graphical representation of the gene expression profile of the 282 b.p. product indicated in FIG. 3 using TOGA analysis (5A-B) and quantitative PCR analysis (5C-D) using RT-PCR primers (SEQ ID NO: 124 and 125).

FIG. 6 is a graphical representation of the results of RT-PCR using 20 pg of clone REC1_{—}1 template, in which amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

FIG. 7 is a graphical representation of the results of RT-PCR using 500 pg of clone REC1_{—}2 template, in which amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

FIG. 8 is a graphical representation of the results of RT-PCR using 500 pg of clone REC1_{—}3 template, in which amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, (large filled squares, “E”), hypercholesterolemic, eight weeks (filled squares, “G”) and hypercholesterolemic plus lercanidipine racemate, eight weeks (filled triangles, “F”).

FIG. 9 is a graphical representation of the results of RT-PCR using 20 pg of clone REC1_{—}8 template, in which amount of PCR product (measured in arbitrary fluorescence units) is plotted against number of cycles, where polyA enriched mRNA was extracted from aortas of rabbits that were normocholesterolermic (filled diamonds, “H2”), hypercholesterolemic, eight weeks (filled squares, “G”) and hypercholesterolemic plus lercanidipine racemate, eight weeks (filled triangles, “F”).

FIG. 10 is a graphical representation of the results of RT-PCR using 100 pg of clone REC1_{—}10 template, in which amount of PCR product (measured in arbitrary fluorescence units) is plotted against number of cycles, where polyA enriched mRNA was extracted from aortas of rabbits that were normocholesterolemic (filled diamonds, “H2”) and hypercholesterolemic, (filled squares, “E”).

FIG. 11 is a graphical representation of the results of RT-PCR using 20 pg of clone REC1_{—}6 template, in which amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

FIG. 12 is a graphical representation of the results of RT-PCR using 100 pg of clone REC1_{—}13 template, in which amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

FIG. 13 is a graphical representation of the results of RT-PCR using 20 pg of clone REC1_{—}18 template, in which amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

FIG. 14 is a graphical representation of the results of RT-PCR using 100 pg of clone REC1_{—}7 template, in which amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, hypercholesterolemic, eight weeks (filled squares, “G”) and hypercholesterolemic plus lercanidipine racemate, eight weeks (filled triangles, “F”).

FIG. 15 is a graphical representation of the results of RT-PCR using 100 pg of clone REC1_{—}5 template, in which amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

FIG. 16 is a graphical representation of the results of RT-PCR using 20 pg of clone REC1_{—}16 template, in which amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

FIG. 17 is a graphical representation of the results of RT-PCR using 20 pg of clone REC1_{—}17 template, in which amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

FIG. 18 is a graphical representation of the results of RT-PCR using 100 pg of clone REC1_{—}19 template, in which amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

FIG. 19 is a graphical representation of the results of RT-PCR using 20 pg of clone REC1_{—}20 template, in which amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

FIG. 20 is a graphical representation of the results of RT-PCR using 20 pg of clone REC1_{—}21 template, in which amount of PCR product (measured in arbitrary fluorescence units) is plotted against number of cycles, where polyA enriched mRNA was extracted from aortas of rabbits that were normocholesterolemic (filled diamonds, “H2”) and hypercholesterolemic plus lercanidipine racemate, eight weeks (filled squares, “F”).

FIG. 21 is a graphical representation of the results of RT-PCR using 20 pg of clone REC1_{—}12 template, in which amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

FIG. 22 is a graphical representation of the results of RT-PCR using 20 pg of clone REC1_{—}22 template, in which amount of PCR product (measured in arbitrary fluorescence units) is plotted against number of cycles, where polyA enriched mRNA was extracted from aortas of rabbits that were normocholesterolemic (filled diamonds, “H2”) and hypercholesterolemic plus lercanidipine R(−), eight weeks (filled squares, “D”).

FIG. 23 is a graphical representation of the results of RT-PCR using 100 pg of clone REC1_{—}24 template, in which amount of PCR product (measured in arbitrary fluorescence units) is plotted against number of cycles, where polyA enriched mRNA was extracted from aortas of rabbits that were normocholesterolemic (filled diamonds, “H2”) and hypercholesterolemic plus lercanidipine R(−), eight weeks (filled squares, “D”).

FIG. 24 is a graphical representation of the results of RT-PCR using 100 pg of clone REC1_{—}36 template, in which amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

### DETAILED DESCRIPTION OF THE PREFERRED EMBODIMENTS

### Definitions

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide.

In the present invention, a “secreted” protein refers to those proteins capable of being directed to the ER, secretory vesicles, or the extracellular space as a result of a signal sequence, as well as those proteins released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

As used herein, a “polynucleotide” refers to a molecule having a nucleic acid sequence contained in SEQ ID NOs:1-55. For example, the polynucleotide can contain all or part of the nucleotide sequence of the full length cDNA sequence, including the 5′ and 3′ untranslated sequences, the coding region, with or without the signal sequence, the secreted protein coding region, as well as fragments, epitopes, domains, and variants of the nucleic acid sequence. Moreover, as used herein, a “polypeptide” refers to a molecule having the translated amino acid sequence generated from the polynucleotide as broadly defined.

A “polynucleotide” of the present invention also includes those polynucleotides capable of hybridizing, under stringent hybridization conditions, to sequences contained in SEQ ID NOs:1-55, or the complement thereof, or the cDNA. “Stringent hybridization conditions” refers to an overnight incubation at 42° C. in a solution comprising 50% formamide, 5×SSC (750 mM NaCl, 75 mM sodium citrate), 50 mM sodium phosphate (pH 7.6), 5× Denhardt's solution, 10% dextran sulfate, and 20 mg/ml denatured, sheared salmon sperm DNA, followed by washing the filters in 0.1×SSC at about 65° C.

Also contemplated are nucleic acid molecules that hybridize to the polynucleotides of the present invention at lower stringency hybridization conditions. Changes in the stringency of hybridization and signal detection are primarily accomplished through the manipulation of formamide concentration (lower percentages of formamide result in lowered stringency); salt conditions, or temperature. For example, lower stringency conditions include an overnight incubation at 37° C. in a solution comprising 6×SSPE (20×SSPE=3M NaCl; 0.2M NaH₂PO₄; 0.02M EDTA, pH 7.4), 0.5% SDS, 30% formamide, 100 ug/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC).

Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Of course, a polynucleotide which hybridizes only to polyA+ sequences (such as any 3′ terminal polyA+ tract of a cDNA shown in the sequence listing), or to a complementary stretch of T (or U) residues, would not be included in the definition of “polynucleotide,” since such a polynucleotide would hybridize to any nucleic acid molecule containing a poly (A) stretch or the complement thereof (e.g., practically any double-stranded cDNA clone).

A polynucleotide of the present invention can be composed of any polyribonucleotide or polydeoxribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, a polynucleotide can be composed of triple-stranded regions comprising RNA or DNA or both RNA and DNA. A polynucleotide may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The polypeptide of the present invention can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formulation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, e.g., T. E. Creighton, Ed., Proteins—Structure And Molecular Properties, 2nd Ed., W.H. Freeman and Company, New York (1993); B. C. Johnson, Ed., Posttranslational Covalent Modification Of Proteins, Academic Press, New York, pgs. 1-12 (1983); Seifter et al., Meth. Enzymol., 182:626-646 (1990); Rattan et al., Ann. N.Y. Acad. Sci., 663:48-62 (1992)).

“A polypeptide having biological activity” refers to polypeptides exhibiting activity similar, but not necessarily identical to, an activity of a polypeptide of the present invention, including mature forms, as measured in a particular biological assay, with or without dose-dependency. In the case where dose dependency does exist, it need not be identical to that of the polypeptide, but rather substantially similar to the dose-dependence in a given activity as compared to the polypeptide of the present invention (i.e., the candidate polypeptide will exhibit greater activity or not more than about 25-fold less and, preferably, not more than about ten-fold less activity and, most preferably, not more than about three-fold less activity relative to the polypeptide of the present invention).

The translated amino acid sequence, beginning with the methionine, is identified although other reading frames can also be easily translated using known molecular biology techniques. The polypeptides produced by the translation of these alternative open reading frames are specifically contemplated by the present invention.

SEQ ID NOs:1-55 and the translations of SEQ ID NOs:1-55 are sufficiently accurate and otherwise suitable for a variety of uses well known in the art and described further below. These nucleic acid molecules will also hybridize to nucleic acid molecules in biological samples, thereby enabling a variety of forensic and diagnostic methods of the invention. Similarly, polypeptides identified from the translations of SEQ ID NOs:1-55 may be used to generate antibodies which bind specifically to the secreted proteins encoded by the cDNA clones identified.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1,000 bases).

The present invention also relates to the genes corresponding to SEQ ID NOs:1-55, and translations of SEQ ID NOs:1-55. The corresponding gene can be isolated in accordance with known methods using the sequence information disclosed herein. Such methods include preparing probes or primers from the disclosed sequence and identifying or amplifying the corresponding gene from appropriate sources of genomic material.

Also provided in the present invention are species homologues. Species homologues may be isolated and identified by making suitable probes or primers from the sequences provided herein and screening a suitable nucleic acid source for the desired homologue.

The polypeptides of the invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

The polypeptides may be in the form of the secreted protein, including the mature form, or may be a part of a larger protein, such as a fusion protein (see below). It is often advantageous to include an additional amino acid sequence which contains secretory or leader sequences, pro-sequences, sequences which aid in purification (such as multiple histidine residues), or an additional sequence for stability during recombinant production.

The polypeptides of the present invention are preferably provided in an isolated form, and preferably are substantially purified. A recombinantly produced version of a polypeptide, including the secreted polypeptide, can be substantially purified by the one-step method described in Smith & Johnson, Gene, 67:31-40 (1988). Polypeptides of the invention also can be purified from natural or recombinant sources using antibodies of the invention raised against the secreted protein in methods which are well known in the art.

Signal Sequences

Methods for predicting whether a protein has a signal sequence, as well as the cleavage point for that sequence, are available. For instance, the method of McGeoch uses the information from a short N-terminal charged region and a subsequent uncharged region of the complete (uncleaved) protein (Virus Res., 3:271-286 (1985)). The method of von Heinje uses the information from the residues surrounding the cleavage site, typically residues −13 to +2, where +1 indicates the amino terminus of the secreted protein (Nucleic Acids Res., 14:4683-4690 (1986)). Therefore, from a deduced amino acid sequence, a signal sequence and mature sequence can be identified.

In the present case, the deduced amino acid sequence of the secreted polypeptide was analyzed by a computer program called Signal P (Nielsen et al., Protein Engineering, 10: 1-6 (1997), which predicts the cellular location of a protein based on the amino acid sequence. As part of this computational prediction of localization, the methods of McGeoch and von Heinje are incorporated.

As one of ordinary skill would appreciate, however, cleavage sites sometimes vary from organism to organism and cannot be predicted with absolute certainty. Accordingly, the present invention provides secreted polypeptides having a sequence corresponding to the translations of SEQ ID NOs:1-55 which have an N-terminus beginning within 5 residues (i.e., + or −5 residues) of the predicted cleavage point. Similarly, it is also recognized that in some cases, cleavage of the signal sequence from a secreted protein is not entirely uniform, resulting in more than one secreted species. These polypeptides, and the polynucleotides encoding such polypeptides, are contemplated by the present invention.

Moreover, the signal sequence identified by the above analysis may not necessarily predict the naturally occurring signal sequence. For example, the naturally occurring signal sequence may be further upstream from the predicted signal sequence. However, it is likely that the predicted signal sequence will be capable of directing the secreted protein to the ER. These polypeptides, and the polynucleotides encoding such polypeptides, are contemplated by the present invention.

Polynucleotide and Polypeptide Variants

“Variant” refers to a polynucleotide or polypeptide differing from the polynucleotide or polypeptide of the present invention, but retaining essential properties thereof. In general, variants have close similarity overall and are identical in many regions to the polynucleotide or polypeptide of the present invention.

“Identity” per se has an art-recognized meaning and can be calculated using published techniques. (See, e.g., Lesk, Ed., Computational Molecular Biology, Oxford University Press, New York, (1988); Smith, Ed., Biocomputing: Informatics And Genome Projects, Academic Press, New York, (1993); Griffin and Griffin, Eds., Computer Analysis Of Sequence Data, Part I, Humana Press, New Jersey, (1994); von Heinje, Sequence Analysis In Molecular Biology, Academic Press, (1987); and Gribskov and Devereux, Eds., Sequence Analysis Primer, M Stockton Press, New York, (1991)). While there exists a number of methods to measure identity between two polynucleotide or polypeptide sequences, the term “identity” is well known to skilled artisans (Carillo et al., SIAM J Applied Math., 48:1073 (1988)). Methods commonly employed to determine identity or similarity between two sequences include, but are not limited to, those disclosed in “Guide to Huge Computers,” Martin J. Bishop, Ed., Academic Press, San Diego, (1994) and Carillo et al., (1988), Supra. Methods for aligning polynucleotides or polypeptides are codified in computer programs, including the GCG program package (Devereux et al., Nuc. Acids Res. 12:387 (1984)), BLASTP, BLASTN, FASTA (Atschul et al., J. Molec. Biol. 215:403 (1990)), and Bestfit program (Wisconsin Sequence Analysis Package, Version 8 for Unix, Genetics Computer Group, University Research Park, 575 Science Drive, Madison, Wis. 53711) which uses the local homology algorithm of Smith and Waterman (Adv. in App. Math., 2:482-489 (1981)).

When using any of the sequence alignment programs to determine whether a particular sequence is, for instance, 95% identical to a reference sequence, the parameters are set such that the percentage of identity is calculated over the full length of the reference polynucleotide and that gaps in identity of up to 5% of the total number of nucleotides in the reference polynucleotide are allowed.

A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci., 6:237-245 (1990)). The term “sequence” includes nucleotide and amino acid sequences. In a sequence alignment the query and subject sequences are either both nucleotide sequences or both amino acid sequences. The result of said global sequence alignment is presented in terms of percent identity. Preferred parameters used in a FASTDB search of a DNA sequence to calculate percent identity are: Matrix=Unitary, k-tuple=4, Mismatch Penalty=1, Joining Penalty=30, Randomization Group Length=0, and Cutoff Score=1, Gap Penalty=5, Gap Size Penalty 0.05, and Window Size=500 or query sequence length in nucleotide bases, whichever is shorter. Preferred parameters employed to calculate percent identity and similarity of an amino acid alignment are: Matrix=PAM 150, k-tuple=2, Mismatch Penalty=1, Joining Penalty=20, Randomization Group Length=0, Cutoff Score=1, Gap Penalty=5, Gap Size Penalty=0.05, and Window Size=500 or query sequence length in amino acid residues, whichever is shorter.

As an illustration, a polynucleotide having a nucleotide sequence of at least 95% “identity” to a sequence contained in SEQ ID NOs:1-55 means that the polynucleotide is identical to a sequence contained in SEQ ID NOs:1-55 or the cDNA except that the polynucleotide sequence may include up to five point mutations per each 100 nucleotides of the total length (not just within a given 100 nucleotide stretch). In other words, to obtain a polynucleotide having a nucleotide sequence at least 95% identical to SEQ ID NOs:1-55, up to 5% of the nucleotides in the sequence contained in SEQ ID NOs:1-55 or the cDNA can be deleted, inserted, or substituted with other nucleotides. These changes may occur anywhere throughout the polynucleotide.

Further embodiments of the present invention include polynucleotides having at least 80% identity, more preferably at least 90% identity, and most preferably at least 95%, 96%, 97%, 98% or 99% identity to a sequence contained in SEQ ID NOs:1-55. Of course, due to the degeneracy of the genetic code, one of ordinary skill in the art will immediately recognize that a large number of the polynucleotides having at least 85%, 90%, 95%, 96%, 97%, 98%, or 99% identity will encode a polypeptide identical to an amino acid sequence contained in the translations of SEQ ID NOs:1-55.

Similarly, by a polypeptide having an amino acid sequence having at least, for example, 95% “identity” to a reference polypeptide, is intended that the amino acid sequence of the polypeptide is identical to the reference polypeptide except that the polypeptide sequence may include up to five amino acid alterations per each 100 amino acids of the total length of the reference polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

Further embodiments of the present invention include polypeptides having at least 80% identity, more preferably at least 85% identity, more preferably at least 90% identity, and most preferably at least 95%, 96%, 97%, 98% or 99% identity to an amino acid sequence contained in translations of SEQ ID NOs:1-55. Preferably, the above polypeptides should exhibit at least one biological activity of the protein.

In a preferred embodiment, polypeptides of the present invention include polypeptides having at least 90% similarity, more preferably at least 95% similarity, and still more preferably at least 96%, 97%, 98%, or 99% similarity to an amino acid sequence contained in translations of SEQ ID NOs:1-55.

The variants may contain alterations in the coding regions, non-coding regions, or both. Especially preferred are polynucleotide variants containing alterations that produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. Polynucleotide variants can be produced for a variety of reasons. For instance, a polynucleotide variant may be produced to optimize codon expression for a particular host (i.e., codons in the human mRNA may be changed to those preferred by a bacterial host, such as E. coli).

Naturally occurring variants are called “allelic variants,” and refer to one of several alternate forms of a gene occupying a given locus on a chromosome of an organism (Lewin, Ed., Genes II, John Wiley & Sons, New York (1985)). These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of the polypeptides of the present invention. For instance, one or more amino acids can be deleted from the N-terminus or C-terminus of the secreted protein without substantial loss of biological function. Ron et al. reported variant KGF proteins having heparin binding activity even after deleting 3, 8, or 27 amino-terminal amino acid residues (J. Biol. Chem. 268: 2984-2988 (1993)). Similarly, interferon gamma exhibited up to ten times higher activity after deleting 8-10 amino acid residues from the carboxy terminus of this protein (Dobeli et al., J. Biotechnology, 7:199-216 (1988)).

Moreover, ample evidence demonstrates that variants often retain a biological activity similar to that of the naturally occurring protein. For example, Gayle et al. conducted extensive mutational analysis of human cytokine IL-1a (J. Biol. Chem., 268:22105-22111 (1993)). These investigators used random mutagenesis to generate over 3,500 individual IL-1 a mutants that averaged 2.5 amino acid changes per variant over the entire length of the molecule. Multiple mutations were examined at every possible amino acid position. The investigators concluded that “[m]ost of the molecule could be altered with little effect on either [binding or biological activity].” (See Gayle et al. (1993), Abstract). In fact, only 23 unique amino acid sequences, out of more than 3,500 amino acid sequences examined, produced a protein that differed significantly in activity from the wild-type sequence.

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

Thus, the invention further includes polypeptide variants which show substantial biological activity. Such variants include deletions, insertions, inversions, repeats, and substitutions selected according to general rules known in the art so as have little effect on activity. For example, guidance concerning how to make phenotypically silent amino acid substitutions is provided in Bowie et al., Science, 247:1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, the amino acid positions which have been conserved between species can be identified. These conserved amino acids are likely important for protein function. In contrast, the amino acid positions in which substitutions have been tolerated by natural selection indicate positions which are not critical for protein function. Thus, positions tolerating amino acid substitution may be modified while still maintaining biological activity of the protein.

The second strategy uses genetic engineering to introduce amino acid changes at specific positions of a cloned gene to identify regions critical for protein function. For example, site-directed mutagenesis or alanine-scanning mutagenesis (the introduction of single alanine mutations at every residue in the molecule) can be used (Cunningham et al., Science, 244:1081-1085 (1989)). The resulting mutant molecules can then be tested for biological activity.

According to Bowie et al., these two strategies have revealed that proteins are surprisingly tolerant of amino acid substitutions. The authors further indicate which amino acid changes are likely to be permissive at certain amino acid positions in the protein. For example, the most buried or interior (within the tertiary structure of the protein) amino acid residues require nonpolar side chains, whereas few features of surface or exterior side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln; replacement of the basic residues Lys, Arg, and His; replacement of the aromatic residues Phe, Tyr, and Trp; and replacement of the small-sized amino acids Ala, Ser, Thr, Met, and Gly.

Besides conservative amino acid substitution, variants of the present invention include: (i) substitutions with one or more of the non-conserved amino acid residues, where the substituted amino acid residues may or may not be one encoded by the genetic code; (ii) substitution with one or more of amino acid residues having a substituent group; (iii) fusion of the mature polypeptide with another compound, such as a compound to increase the stability and/or solubility of the polypeptide (e.g., polyethylene glycol); (iv) fusion of the polypeptide with additional amino acids, such as an IgG Fc fusion region peptide, a leader or secretory sequence, or a sequence facilitating purification. Such variant polypeptides are deemed to be within the scope of those skilled in the art from the teachings herein.

For example, polypeptide variants containing amino acid substitutions of charged amino acids with other charged or neutral amino acids may produce proteins with improved characteristics, such as decreased aggregation. As known, aggregation of pharmaceutical formulations both reduces activity and increases clearance due to the aggregate's immunogenic activity (see, e.g., Pinckard et al., Clin. Exp. Immunol. 2:331-340 (1967); Robbins et al., Diabetes, 36: 838-845 (1987); Cleland et al., Crit. Rev. Therap. Drug Carrier Sys., 10:307-377 (1993)).

Polynucleotide and Polypeptide Fragments

In the present invention, a “polynucleotide fragment” refers to a short polynucleotide having a nucleic acid sequence contained in that shown in SEQ ID NOs:1-55. The short nucleotide fragments are preferably at least about 15 nucleotides (nt), and more preferably at least about 20 nt, still more preferably at least about 30 nt, and even more preferably, at least about 40 nt in length. A fragment “at least 20 nt in length,” for example, is intended to include 20 or more contiguous bases from the cDNA sequence contained in that shown in SEQ ID NOs:1-55. These nucleotide fragments are useful as diagnostic probes and primers as discussed herein. Of course, larger fragments (e.g., 50, 150, and greater than 150 nucleotides) are preferred.

Moreover, representative examples of polynucleotide fragments of the invention, include, for example, fragments having a sequence from about nucleotide number 1-50, 51-100, 101-150, 151-200, 201-250, 251-300,301-350, 351-400, 401-450, to the end of SEQ ID NOs:1-55. In this context “about” includes the particularly recited ranges, larger or smaller by several nucleotides (i.e., 5, 4, 3, 2, or 1 nt) at either terminus or at both termini. Preferably, these fragments encode a polypeptide which has biological activity.

In the present invention, a “polypeptide fragment” refers to a short amino acid sequence contained in the translations of SEQ ID NOs:1-55. Protein fragments may be “free-standing,” or comprised within a larger polypeptide of which the fragment forms a part or region, most preferably as a single continuous region. Representative examples of polypeptide fragments of the invention include, for example, fragments from about amino acid number 1-20, 21-40, 41-60, or 61 to the end of the coding region. Moreover, polypeptide fragments can be about 20, 30, 40, 50, or 60 amino acids in length. In this context “about” includes the particularly recited ranges, larger or smaller by several amino acids (5, 4, 3, 2, or 1) at either extreme or at both extremes.

Preferred polypeptide fragments include the secreted protein as well as the mature form. Further preferred polypeptide fragments include the secreted protein or the mature form having a continuous series of deleted residues from the amino or the carboxy terminus, or both. For example, any number of amino acids ranging from 1-60, can be deleted from the amino terminus of either the secreted polypeptide or the mature form. Similarly, any number of amino acids ranging from 1-30, can be deleted from the carboxy terminus of the secreted protein or mature form. Furthermore, any combination of the above amino and carboxy terminus deletions are preferred. Similarly, polynucleotide fragments encoding these polypeptide fragments are also preferred.

Also preferred are polypeptide and polynucleotide fragments characterized by structural or functional domains, such as fragments that comprise alpha-helix and alpha-helix-forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Polypeptide fragments of the translations of SEQ ID NOs:1-55 falling within conserved domains are specifically contemplated by the present invention. Moreover, polynucleotide fragments encoding these domains are also contemplated.

Other preferred fragments are biologically active fragments. Biologically active fragments are those exhibiting activity similar, but not necessarily identical, to an activity of the polypeptide of the present invention. The biological activity of the fragments may include an improved desired activity, or a decreased undesirable activity.

Epitopes & Antibodies

In the present invention, “epitopes” refer to polypeptide fragments having antigenic or immunogenic activity in an animal, especially in a human. A preferred embodiment of the present invention relates to a polypeptide fragment comprising an epitope, as well as the polynucleotide encoding this fragment. A region of a protein molecule to which an antibody can bind is defined as an “antigenic epitope.” In contrast, an “immunogenic epitope” is defined as a part of a protein that elicits an antibody response. (See, e.g., Geysen et al., Proc. Natl. Acad. Sci. USA, 81:3998-4002 (1983)).

Fragments which function as epitopes may be produced by any conventional means. (See, e.g., Houghten, R. A., Proc. Natl. Acad. Sci. USA, 82:5131-5135 (1985), further described in U.S. Pat. No. 4,631,211).

In the present invention, antigenic epitopes preferably contain a sequence of at least seven, more preferably at least nine, and most preferably between about 15 to about 30 amino acids. Antigenic epitopes are useful to raise antibodies, including monoclonal antibodies, that specifically bind the epitope. (See, e.g., Wilson et al., Cell, 37:767-778 (1984); Sutcliffe et al., Science, 219:660-666 (1983)).

Similarly, immunogenic epitopes can be used to induce antibodies according to methods well known in the art. (See, e.g., Sutcliffe et al., (1983) Supra; Wilson et al., (1984) Supra; Chow et al., Proc. Natl. Acad. Sci., USA, 82:910-914; and Bittle et al., J. Gen. Virol., 66:2347-2354 (1985)). A preferred immunogenic epitope includes the secreted protein. The immunogenic epitope may be presented together with a carrier protein, such as an albumin, to an animal system (such as rabbit or mouse). Alternatively, the immunogenic epitope may be prescribed without a carrier, if the sequence is of sufficient length (at least about 25 amino acids). However, immunogenic epitopes comprising as few as 8 to 10 amino acids have been shown to be sufficient to raise antibodies capable of binding to, at the very least, linear epitopes in a denatured polypeptide (e.g., in Western blotting.)

As used herein, the term “antibody” (Ab) or “monoclonal antibody” (Mab) is meant to include intact molecules as well as antibody fragments (such as, for example, Fab and F(ab′)₂ fragments) which are capable of specifically binding to protein. Fab and F(ab′)₂ fragments lack the Fc fragment of intact antibody, clear more rapidly from the circulation, and may have less non-specific tissue binding than an intact antibody (Wahl et al., J. Nucl. Med., 24:316-325 (1983)). Thus, these fragments are preferred, as well as the products of a Fab or other immunoglobulin expression library. Moreover, antibodies of the present invention include chimeric, single chain, and human and humanized antibodies.

Additional embodiments include chimeric antibodies, e.g., humanized versions of murine monoclonal antibodies. Such humanized antibodies may be prepared by known techniques, and offer the advantage of reduced immunogenicity when the antibodies are administered to humans. In one embodiment, a humanized monoclonal antibody comprises the variable region of a murine antibody (or just the antigen binding site thereof) and a constant region derived from a human antibody. Alternatively, a humanized antibody fragment may comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (Nature, 332:323, 1988), Liu et al. (PNAS, 84:3439, 1987), Larrick et al. (Bio/Technology, 7:934, 1989), and Winter and Harris (TIPS, 14:139, May, 1993).

One method for producing a human antibody comprises immunizing a non-human animal, such as a transgenic mouse, with a polypeptide translated from a nucleotide sequence chosen from SEQ ID NOs:1-55, whereby antibodies directed against the polypeptide translated from a nucleotide sequence chosen from SEQ ID NOs:1-55 are generated in said animal. Procedures have been developed for generating human antibodies in non-human animals. The antibodies may be partially human, or preferably completely human. Non-human animals (such as transgenic mice) into which genetic material encoding one or more human immunoglobulin chains has been introduced may be employed. Such transgenic mice may be genetically altered in a variety of ways. The genetic manipulation may result in human immunoglobulin polypeptide chains replacing endogenous immunoglobulin chains in at least some (preferably virtually all) antibodies produced by the animal upon immunization. Antibodies produced by immunizing transgenic animals with a polypeptide translated from a nucleotide sequence chosen from SEQ ID NOs:1-55 are provided herein.

Mice in which one or more endogenous immunoglobulin genes are inactivated by various means have been prepared. Human immunoglobulin genes have been introduced into the mice to replace the inactivated mouse genes. Antibodies produced in the animals incorporate human immunoglobulin polypeptide chains encoded by the human genetic material introduced into the animal. Examples of techniques for production and use of such transgenic animals are described in U.S. Pat. Nos. 5,814,318, 5,569,825, and 5,545,806, which are incorporated by reference herein.

Monoclonal antibodies may be produced by conventional procedures, e.g., by immortalizing spleen cells harvested from the transgenic animal after completion of the immunization schedule. The spleen cells may be fused with myeloma cells to produce hybridomas by conventional procedures.

A method for producing a hybridoma cell line comprises immunizing such a transgenic animal with an immunogen comprising at least seven contiguous amino acid residues of a polypeptide translated from a nucleotide sequence chosen from SEQ ID NOs:1-55; harvesting spleen cells from the immunized animal; fusing the harvested spleen cells to a myeloma cell line, thereby generating hybridoma cells; and identifying a hybridoma cell line that produces a monoclonal antibody that binds a polypeptide translated from a nucleotide sequence chosen from SEQ ID NOs:1-55. Such hybridoma cell lines, and monoclonal antibodies produced therefrom, are encompassed by the present invention. Monoclonal antibodies secreted by the hybridoma cell line are purified by conventional techniques.

Antibodies may be employed in an in vitro procedure, or administered in vivo to inhibit biological activity induced by a polypeptide translated from a nucleotide sequence chosen from SEQ ID NOs:1-55. Disorders caused or exacerbated (directly or indirectly) by the interaction of such polypeptides of the present invention with cell surface receptors thus may be treated. A therapeutic method involves in vivo administration of a blocking antibody to a mammal in an amount effective for reducing a biological activity induced by a polypeptide translated from a nucleotide sequence chosen from SEQ ID NOs:1-55. For example, antibody blockade of the LDL lipoproteins to receptors at sites of endothelium injury may inhibit the formation of atherosclerotic plaques. Similarly, antibody blockade of macrophage or platelet adhesion to endothelial lesions may block the initiation of plaque formation.

Also provided herein are conjugates comprising a detectable (e.g., diagnostic) or therapeutic agent, attached to an antibody directed against a polypeptide translated from a nucleotide sequence chosen from SEQ ID NOs:1-55. Examples of such agents are well known, and include but are not limited to diagnostic radionuclides, therapeutic radionuclides, and cytotoxic drugs. The conjugates find use in in vitro or in vivo procedures.

Fusion Proteins

Any polypeptide of the present invention can be used to generate fusion proteins. For example, the polypeptide of the present invention, when fused to a second protein, can be used as an antigenic tag. Antibodies raised against the polypeptide of the present invention can be used to indirectly detect the second protein by binding to the polypeptide. Moreover, because secreted proteins target cellular locations based on trafficking signals, the polypeptides of the present invention can be used as targeting molecules once fused to other proteins.

Examples of domains that can be fused to polypeptides of the present invention include not only heterologous signal sequences, but also other heterologous functional regions. The fusion does not necessarily need to be direct, but may occur through linker sequences.

Moreover, fusion proteins may also be engineered to improve characteristics of the polypeptide of the present invention. For instance, a region of additional amino acids, particularly charged amino acids, may be added to the N-terminus of the polypeptide to improve stability and persistence during purification from the host cell or subsequent handling and storage. Also, peptide moieties may be added to the polypeptide to facilitate purification. Such regions may be removed prior to final preparation of the polypeptide. The addition of peptide moieties to facilitate handling of polypeptides are familiar and routine techniques in the art.

In addition, polypeptides of the present invention, including fragments and, specifically, epitopes, can be combined with parts of the constant domain of immunoglobulins (IgG), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins (EP A 394,827; Traunecker et al., Nature, 331:84-86 (1988).) Fusion proteins having disulfide-linked dimeric structures (due to the IgG) can also be more efficient in binding and neutralizing other molecules, than the monomeric secreted protein or protein fragment alone (Fountoulakis et al., J. Biochem., 270:3958-3964 (1995)).

Similarly, EP A 0 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and thus can result in, for example, improved pharmacokinetic properties (see, e.g., EP A 0 232 262). Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5 (See, Bennett et al., J. Mol. Recognition 8:52-58 (1995); Johanson et al., J. Biol. Chem., 270:9459-9471 (1995)).

Moreover, the polypeptides of the present invention can be fused to marker sequences, such as a peptide which facilitates purification of the fused polypeptide. In preferred embodiments, the marker amino acid sequence is a hexa-histidine peptide, such as the tag provided in a pQE vector (QIAGEN, Inc., Chatsworth, Calif.), among others, many of which are commercially available. As described in Gentz et al., for instance, hexa-histidine provides for convenient purification of the fusion protein (Proc. Natl. Acad. Sci. USA 86:821-824 (1989)). Another peptide tag useful for purification, the “HA” tag, corresponds to an epitope derived from the influenza hemagglutinin protein (Wilson et al., Cell, 37:767 (1984)). Other fusion proteins may use the ability of the polypeptides of the present invention to target the delivery of a biologically active peptide. This might include focused delivery of a toxin to tumor cells, or a growth factor to stem cells.

Thus, any of these above fusions can be engineered using the polynucleotides or the polypeptides of the present invention.

Vectors, Host Cells, and Protein Production

The present invention also relates to vectors containing the polynucleotide of the present invention, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293, and Bowes melanoma cells, and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, PNH16A, PNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may, in fact, be expressed by a host cell lacking a recombinant vector.

A polypeptide of this invention can be recovered and purified from recombinant cell cultures by well-known methods, including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. Most preferably, high performance liquid chromatography (“HPLC”) is employed for purification.

Polypeptides of the present invention, and preferably the secreted form, can also be recovered from products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

Uses of the Polynucleotides

Each of the polynucleotides identified herein can be used in numerous ways as reagents. The following description should be considered exemplary and utilizes known techniques.

The polynucleotides of the present invention are useful for chromosome identification. There exists an ongoing need to identify new chromosome markers, since few chromosome marking reagents based on actual sequence data (repeat polymorphisms) are presently available. Each polynucleotide of the present invention can be used as a chromosome marker.

Briefly, sequences can be mapped to chromosomes by preparing PCR primers (preferably 15-25 bp) from the sequences shown in SEQ ID NOs:1-55. Primers can be selected using computer analysis so that primers do not span more than one predicted exon in the genomic DNA. These primers are then used for PCR screening of somatic cell hybrids containing individual human chromosomes. Only those hybrids containing the human gene corresponding to the SEQ ID NOs:1-55 will yield an amplified fragment.

Similarly, somatic hybrids provide a rapid method of PCR mapping the polynucleotides to particular chromosomes. Three or more clones can be assigned per day using a single thermal cycler. Moreover, sublocalization of the polynucleotides can be achieved with panels of specific chromosome fragments. Other gene-mapping strategies that can be used include in situ hybridization, prescreening with labeled flow-sorted chromosomes, and preselection by hybridization to construct chromosome specific-cDNA libraries.

Precise chromosomal location of the polynucleotides can also be achieved using fluorescence in situ hybridization (FISH) of a metaphase chromosomal spread. This technique uses polynucleotides as short as 500 or 600 bases; however, polynucleotides of 2,000-4,000 bp are preferred. For a review of this technique, see Verma et al., Human Chromosomes: a Manual of Basic Techniques, Pergamon Press, New York (1988).

For chromosome mapping, the polynucleotides can be used individually (to mark a single chromosome or a single site on that chromosome) or in panels (for marking multiple sites and/or multiple chromosomes). Preferred polynucleotides correspond to the noncoding regions of the cDNAs because the coding sequences are more likely conserved within gene families, thus increasing the chance of cross-hybridization during chromosomal mapping.

Once a polynucleotide has been mapped to a precise chromosomal location, the physical position of the polynucleotide can be used in linkage analysis. Linkage analysis establishes coinheritance between a chromosomal location and presentation of a particular disease. Disease mapping data are found, for example, in V. McKusick, Mendelian Inheritance in Man (available on line through Johns Hopkins University Welch Medical Library)). Assuming one megabase mapping resolution and one gene per 20 kb, a cDNA precisely localized to a chromosomal region associated with the disease could be one of 50-500 potential causative genes.

Thus, once coinheritance is established, differences in the polynucleotide and the corresponding gene between affected and unaffected individuals can be examined. The polynucleotides of SEQ ID NOs:1-55 can be used for this analysis of individuals. Some genes may be associated with susceptibility to atherosclerosis. These may be indirect, through associations with risk factors such as diabetes, or direct, through genetic defects in lipid or cholesterol metabolism. These can be used as markers to identify individuals with susceptibility to atherosclerosis.

First, visible structural alterations in the chromosomes, such as deletions or translocations, are examined in chromosome spreads or by PCR. If no structural alterations exist, the presence of point mutations are ascertained. Mutations observed in some or all affected individuals, but not in normal individuals, indicates that the mutation may cause the disease. However, complete sequencing of the polypeptide and the corresponding gene from several normal individuals is required to distinguish the mutation from a polymorphism. If a new polymorphism is identified, this polymorphic polypeptide can be used for further linkage analysis.

Furthermore, increased or decreased expression of the gene in affected individuals as compared to unaffected individuals can be assessed using polynucleotides of the present invention. Any of these alterations (altered expression, chromosomal rearrangement, or mutation) can be used as a diagnostic or prognostic marker.

In addition to the foregoing, a polynucleotide can be used to control gene expression through triple helix formation or antisense DNA or RNA. Both methods rely on binding of the polynucleotide to DNA or RNA. For these techniques, preferred polynucleotides are usually 20 to 40 bases in length and complementary to either the region of the gene involved in transcription (see, Lee et al., Nucl. Acids Res., 6:3073 (1979); Cooney et al., Science, 241:456 (1988); and Dervan et al., Science, 251:1360 (1991) for discussion of triple helix formation) or to the mRNA itself (see, Okano, J. Neurochem, 56:560 (1991); and Oligodeoxy-nucleotides as Antisense Inhibitors of Gene Expression, CRC Press, Boca Raton, Fla. (1988) for a discussion of antisense technique). Triple helix formation optimally results in a shut-off of RNA transcription from DNA, while antisense RNA hybridization blocks translation of an mRNA molecule into polypeptide. Both techniques are effective in model systems, and the information disclosed herein can be used to design antisense or triple helix polynucleotides in an effort to treat disease.

Polynucleotides of the present invention are also useful in gene therapy. One goal of gene therapy is to insert a normal gene into an organism having a defective gene, in an effort to correct the genetic defect. The polynucleotides disclosed in the present invention offer a means of targeting such genetic defects in a highly accurate manner. Another goal is to insert a new gene that was not present in the host genome, thereby producing a new trait in the host cell.

The polynucleotides are also useful for identifying individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identifying personnel. This method does not suffer from the current limitations of “Dog Tags” which can be lost, switched, or stolen, making positive identification difficult. The polynucleotides of the present invention can be used as additional DNA markers for RFLP.

The polynucleotides of the present invention can also be used as an alternative to RFLP, by determining the actual base-by-base DNA sequence of selected portions of an individual's genome. These sequences can be used to prepare PCR primers for amplifying and isolating such selected DNA, which can then be sequenced. Using this technique, individuals can be identified because each individual will have a unique set of DNA sequences. Once an unique ID database is established for an individual, positive identification of that individual, living or dead, can be made from extremely small tissue samples.

Forensic biology also benefits from using DNA-based identification techniques as disclosed herein. DNA sequences taken from very small biological samples such as tissues, e.g., hair or skin, or body fluids, e.g., blood, saliva, semen, etc., can be amplified using PCR. In one prior art technique, gene sequences amplified from polymorphic loci, such as DQa class II HLA gene, are used in forensic biology to identify individuals. (Erlich, Ed., PCR Technology, M. Stockton Press (1989)). Once these specific polymorphic loci are amplified, they are digested with one or more restriction enzymes, yielding an identifying set of bands on a Southern blot probed with DNA corresponding to the DQa class H HLA gene. Similarly, polynucleotides of the present invention can be used as polymorphic markers for forensic purposes.

There is also a need for reagents capable of identifying the source of a particular tissue. Such need arises, for example, in forensics when presented with tissue of unknown origin. Appropriate reagents can comprise, for example, DNA probes or primers specific to particular tissue prepared from the sequences of the present invention. Panels of such reagents can identify tissue by species and/or by organ type. In a similar fashion, these reagents can be used to screen tissue cultures for contamination.

In the very least, the polynucleotides of the present invention can be used as molecular weight markers on Southern gels; as diagnostic probes for the presence of a specific mRNA in a particular cell type; as a probe to “subtract-out” known sequences in the process of discovering novel polynucleotides; for selecting and making oligomers for attachment to a “gene chip” or other support; to raise anti-DNA antibodies using DNA immunization techniques; and as an antigen to elicit an immune response.

Uses of the Polypeptides

Each of the polypeptides identified herein can be used in numerous ways. The following description should be considered exemplary and utilizes known techniques.

A polypeptide of the present invention can be used to assay protein levels in a biological sample using antibody-based techniques. For example, protein expression in tissues can be studied with classical immunohistological methods (Jalkanen, et al., J. Cell. Biol., 101:976-985 (1985); Jalkanen, et al., J. Cell Biol., 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as glucose oxidase; and radioisotopes, such as iodine (¹²⁵I, ¹²¹I, carbon (¹⁴C), sulfur (³⁵S), tritium (³H), indium (¹¹²In), and technetium (^{99m}Tc); fluorescent labels, such as fluorescein and rhodamine; and biotin.

In addition to assaying secreted protein levels in a biological sample, proteins can also be detected in vivo by imaging. Antibody labels or markers for in vivo imaging of protein include those detectable by X-radiography, nuclear magnetic resonance (NMR), or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A protein-specific antibody or antibody fragment which has been labeled with an appropriate detectable imaging moiety such as a radioisotope (e.g., ¹³¹I, ¹¹²In, ^{99m}Tc), a radio-opaque substance, or a material detectable by NMR, is introduced (e.g., parenterally, subcutaneously, or intraperitoneally) into the mammal. It will be understood in the art that the size of the subject and the imaging system used will determine the quantity of imaging moiety needed to produce diagnostic images. In the case of a radioisotope moiety, the quantity of radioactivity necessary for a human subject will normally range from about 5 to 20 millicuries of ^{99m}Tc. The labeled antibody or antibody fragment will then preferentially accumulate at the location of cells which contain the specific protein. In vivo tumor imaging is described in Burchiel et al., “Immunopharmacokinetics of Radiolabeled Antibodies and Their Fragments” (Chapter 13 in Tumor Imaging: The Radiochemical Detection of Cancer, Burchiel and Rhodes, Eds., Masson Publishing Inc. (1982)).

Thus, the invention provides a diagnostic method of a disorder, which involves (a) assaying the expression of a polypeptide of the present invention in cells or body fluid of an individual; and (b) comparing the level of gene expression with a standard gene expression level, whereby an increase or decrease in the assayed polypeptide gene expression level compared to the standard expression level is indicative of a disorder. For example, macrophages activated in atherosclerotic lesions may show specific changes in gene expression. If these changes are also found in macrophages circulating in peripheral blood, this may be detected in blood samples from patients.

Moreover, polypeptides of the present invention can be used to treat disease. For example, patients can be administered a polypeptide of the present invention in an effort to replace absent or decreased levels of the polypeptide (e.g., insulin); to supplement absent or decreased levels of a different polypeptide (e.g., hemoglobin S for hemoglobin B); to inhibit the activity of a polypeptide (e.g., an oncogene); to activate the activity of a polypeptide (e.g., by binding to a receptor); to reduce the activity of a membrane bound receptor by competing with it for free ligand (e.g., soluble TNF receptors used in reducing inflammation); or to bring about a desired response (e.g., blood vessel growth).

Similarly, antibodies directed to a polypeptide of the present invention can also be used to treat disease. For example, administration of an antibody directed to a polypeptide of the present invention can bind and reduce overproduction of the polypeptide. Similarly, administration of an antibody can activate the polypeptide, such as by binding to a polypeptide bound to a membrane (receptor). Polypeptides can be used as antigens to trigger immune responses. For example, activated macrophages that are filled with lipid in atherosclerotic lesions may express genes unique to this activated state. Immunization against these markers may stimulate antibody responses or cellular immune responses that could eliminate the lipid-laden macrophages, and eliminate the atherosclerotic lesions.

At the very least, the polypeptides of the present invention can be used as molecular weight markers on SDS-PAGE gels or on molecular sieve gel filtration columns using methods well-known to those of skill in the art. Polypeptides can also be used to raise antibodies, which in turn are used to measure protein expression from a recombinant cell, as a way of assessing transformation of the host cell. Moreover, the polypeptides of the present invention can be used to test the following biological activities.

Biological Activities

The polynucleotides and polypeptides of the present invention can be used in assays to test for one or more biological activities. If these polynucleotides and polypeptides do exhibit activity in a particular assay, it is likely that these molecules may be involved in the diseases associated with the biological activity. Thus, the polynucleotides and polypeptides could be used to treat the associated disease.

Nervous System Activity

A polypeptide or polynucleotide of the present invention may be useful in treating deficiencies or disorders of the central nervous system or peripheral nervous system by activating or inhibiting the proliferation, differentiation, or mobilization (chemotaxis) of neuroblasts, stem cells, or glial cells. Also, a polypeptide or polynucleotide of the present invention may be useful in treating deficiencies or disorders of the central nervous system or peripheral nervous system by activating or inhibiting the mechanisms of synaptic transmission, synthesis, metabolism and inactivation of neural transmitters, neuromodulators and trophic factors, and by activating or inhibiting the expression and incorporation of enzymes, structural proteins, membrane channels, and receptors in neurons and glial cells.

The etiology of these deficiencies or disorders may be genetic, somatic (such as cancer or some autoimmune disorder), acquired (e.g., by chemotherapy or toxins), or infectious. Moreover, a polynucleotide or polypeptide of the present invention can be used as a marker or detector of a particular nervous system disease or disorder. The disorder or disease can be any of Alzheimer's disease, Pick's disease, Binswanger's disease, other senile dementia, Parkinson's disease, parkinsonism, obsessive compulsive disorders, epilepsy, encephaolopathy, ischemia, alcohol addiction, drug addiction, schizophrenia, amyotrophic lateral sclerosis, multiple sclerosis, depression, and bipolar manic-depressive disorder. Alternatively, the polypeptide or polynucleotide of the present invention can be used to study circadian variation, aging, or long-term potentiation, the latter affecting the hippocampus. Additionally, particularly with reference to mRNA species occurring in particular structures within the central nervous system, the polypeptide or polynucleotide of the present invention can be used to study brain regions that are known to be involved in complex behaviors, such as learning and memory, emotion, drug addiction, glutamate neurotoxicity, feeding behavior, olfaction, viral infection, vision, and movement disorders.

Immune Activity

A polypeptide or polynucleotide of the present invention may be useful in treating deficiencies or disorders of the immune system, by activating or inhibiting the proliferation, differentiation, or mobilization (chemotaxis) of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune deficiencies or disorders may be genetic, somatic (such as cancer or some autoimmune disorders) acquired (e.g., by chemotherapy or toxins), or infectious. Moreover, a polynucleotide or polypeptide of the present invention can be used as a marker or detector of a particular immune system disease or disorder.

A polynucleotide or polypeptide of the present invention may be useful in treating or detecting deficiencies or disorders of hematopoictic cells. A polypeptide or polynucleotide of the present invention could be used to increase differentiation and proliferation of hematopoietic cells, including the pluripotent stem cells, in an effort to treat those disorders associated with a decrease in certain (or many) types hematopoietic cells. Examples of immunologic deficiency syndromes include, but are not limited to: blood protein disorders (e.g. agammaglobulinemia, dysgammaglobulinemia), ataxia telangiectasia, common variable immunodeficiency, Di George's Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

Moreover, a polypeptide or polynucleotide of the present invention could also be used to modulate hemostatic (bleeding cessation) or thrombolytic activity (clot formation). For example, by increasing hemostatic or thrombolytic activity, a polynucleotide or polypeptide of the present invention could be used to treat blood coagulation disorders (e.g., afibrinogenemia, factor deficiencies), blood platelet disorders (e.g. thrombocytopenia), or wounds resulting from trauma, surgery, or other causes. Alternatively, a polynucleotide or polypeptide of the present invention that can decrease hemostatic or thrombolytic activity could be used to inhibit or dissolve clotting. These molecules could be important in the treatment of heart attacks (infarction), strokes, or scarring.

A polynucleotide or polypeptide of the present invention may also be useful in the treatment or detection of autoimmune disorders. Many autoimmune disorders result from inappropriate recognition of self as foreign material by immune cells. This inappropriate recognition results in an immune response leading to the destruction of the host tissue. Therefore, the administration of a polypeptide or polynucleotide of the present invention that inhibits an immune response, particularly the proliferation, differentiation, or chemotaxis of T-cells response or in some way results in the induction of tolerance, may be an effective therapy in preventing autoimmune disorders.

Examples of autoimmune disorders that can be treated or detected by the present invention include, but are not limited to: Addison's Disease, hemolytic anemia, antiphospbolipid syndrome, rheumatoid arthritis, dermatitis, allergic encephalomyelitis, glomerulonephritis, Goodpasture's Syndrome, Graves' Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

Similarly, allergic reactions and conditions, such as asthma (particularly allergic asthma) or other respiratory problems, may also be treated by a polypeptide or polynucleotide of the present invention. Moreover, these molecules can be used to treat anaphylaxis, hypersensitivity to an antigenic molecule, or blood group incompatibility.

A polynucleotide or polypeptide of the present invention may also be used to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of a polypeptide or polynucleotide of the present invention that inhibits an immune response, particularly the proliferation, differentiation, or chemotaxis of T-cells, may be an effective therapy in preventing organ rejection or GVHD.

Similarly, a polypeptide or polynucleotide of the present invention may also be used to modulate inflammation. For example, the polypeptide or polynucleotide may inhibit the proliferation and differentiation of cells involved in an inflammatory response. These molecules can be used to treat inflammatory conditions, both chronic and acute conditions, including inflammation associated with infection (e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease, Crohn's disease, or resulting from over production of cytokines (e.g., TNF or IL1.)

Hyperproliferative Disorders

A polypeptide or polynucleotide can be used to treat or detect hyperproliferative disorders, including neoplasms. A polypeptide or polynucleotide of the present invention may inhibit the proliferation of the disorder through direct or indirect interactions. Alternatively, a polypeptide or polynucleotide of the present invention may proliferate other cells which can inhibit the hyperproliferative disorder.

For example, by increasing an immune response, particularly increasing antigenic qualities of the hyperproliferative disorder or by inducing the proliferation, differentiation, or mobilization of T-cells, hyperproliferative disorders can be treated. This immune response may be increased by either enhancing an existing immune response, or by initiating a new immune response. Alternatively, decreasing an immune response may also be a method of treating hyperproliferative disorders, such as by administering the polypeptide or polynucleotide as a chemotherapeutic agent.

Examples of hyperproliferative disorders that can be treated or detected by a polynucleotide or polypeptide of the present invention include, but are not limited to neoplasms located in the abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic region, skin, soft tissue, spleen, thoracic region, and urogenital system.

Similarly, other hyperproliferative disorders can also be treated or detected by a polynucleotide or polypeptide of the present invention. Examples of such hyperproliferative disorders include, but are not limited to hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

Infectious Disease

A polypeptide or polynucleotide of the present invention can be used to treat or detect infectious agents. For example, by increasing the immune response, particularly increasing the proliferation and differentiation of B and/or T cells, infectious diseases may be treated. The immune response may be increased by either enhancing an existing immune response, or by initiating a new immune response. Alternatively, the polypeptide or polynucleotide of the present invention may also directly inhibit the infectious agent, without necessarily eliciting an immune response.

Viruses are one example of an infectious agent that can cause disease or symptoms that can be treated or detected by a polynucleotide or polypeptide of the present invention. Examples of viruses include, but are not limited to, the following DNA and RNA viral families: Arbovirus, Adenoviridae, Arenaviridae, Arterivirus, Birnviridae, Bunyaviridae, Caliciviridae, Circoviridae, Coronaviridae, Flaviviridae, Hepadnaviridae (Hepatitis), Herpesviridae (such as Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picomaviridae, Poxyiridae (such as Smallpox or Vaccinia), Reoviridae (e.g., Rotavirus), Retroviridae (HTLV-1, HTLV-II, Lentivirus), and Togaviridae (e.g., Rubivirus). Viruses falling within these families can cause a variety of diseases or symptoms, including, but not limited to, arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia. A polypeptide or polynucleotide of the present invention can be used to treat or detect any of these symptoms or diseases.

Similarly, bacterial or fungal agents that can cause disease or symptoms and that can be treated or detected by a polynucleotide or polypeptide of the present invention include, but are not limited to, the following Gram-Negative and Gram-positive bacterial families and fungi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsielia, Salmonella, Serratia, Yersinia), Erysipelothrix, Helicobacter, Legionellosis, Leptospirosis, Listeria, Mycoplasmatales, Neisseriaceae (e.g., Acinetobacter, Gonorrhea, Menigococcal), Pasteurellacea infections (e.g., Actinobacillus, Heamophilus, Pasteurella), Pseudomonas, Rickettsiaceae, Chlamydiaceae, Syphilis, and Staphylococcus. These bacterial or fungal families can cause numerous diseases or symptoms, including, but not limited to, bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections (such as whooping Cough or empyema), sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually-transmitted diseases, skin diseases (e.g., cellulitis, dermatocycoses), toxemia, urinary tract infections, and wound infections. A polypeptide or polynucleotide of the present invention can be used to treat or detect any of these symptoms or diseases.

Moreover, parasitic agents causing disease or symptoms that can be treated or detected by a polynucleotide or polypeptide of the present invention include, but are not limited to, the following families: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomoniasis. These parasites can cause a variety of diseases or symptoms, including, but not limited to, Scabies, Trombiculiasis, eye infections, intestinal disease (e.g., dysentery, giardiasis), liver disease, lung disease, opportunistic infections (e.g., AIDS related), Malaria, pregnancy complications, and toxoplasmosis. A polypeptide or polynucleotide of the present invention can be used to treat or detect any of these symptoms or diseases.

Preferably, treatment using a polypeptide or polynucleotide of the present invention could either be by administering an effective amount of a polypeptide to the patient, or by removing cells from the patient, supplying the cells with a polynucleotide of the present invention, and returning the engineered cells to the patient (ex vivo therapy). Moreover, the polypeptide or polynucleotide of the present invention can be used as an antigen in a vaccine to raise an immune response against infectious disease.

Regeneration

A polynucleotide or polypeptide of the present invention can be used to differentiate, proliferate, and attract cells, leading to the regeneration of tissues (see, Science, 276:59-87 (1997)). The regeneration of tissues could be used to repair, replace, or protect tissue damaged by congenital defects, trauma (wounds, burns, incisions, or ulcers), age, disease (e.g. osteoporosis, osteocarthritis, periodontal disease, liver failure), surgery (including cosmetic plastic surgery), fibrosis, reperfusion injury, or systemic cytokine damage.

Tissues that could be regenerated using the present invention include organs (e.g., pancreas, liver, intestine, kidney, skin, endothelium), muscle (smooth, skeletal or cardiac), vascular (including vascular endothelium), nervous, hematopoietic, and skeletal (bone, cartilage, tendon, ligament) tissue. Preferably, regeneration occurs without scarring or with minimal scarring. Regeneration also may include angiogenesis. In the case of atherosclerosis, improper healing of vascular endothelium lesions may be the primary trigger of atherosclerotic plaque formation. Molecules that may induce more efficient wound healing may prevent plaque formation.

Moreover, a polynucleotide or polypeptide of the present invention may increase regeneration of tissues difficult to heal. For example, increased tendon/ligament regeneration would quicken recovery time after damage. A polynucleotide or polypeptide of the present invention could also be used prophylactically in an effort to avoid damage. Specific diseases that could be treated include of tendinitis, carpal tunnel syndrome, and other tendon or ligament defects. A further example of tissue regeneration of non-healing wounds includes pressure ulcers, ulcers associated with vascular insufficiency, surgical, and traumatic wounds.

Similarly, nerve and brain tissue could also be regenerated by using a polynucleotide or polypeptide of the present invention to proliferate and differentiate nerve cells. Diseases that could be treated using this method include central and peripheral nervous system diseases, neuropathies, or mechanical and traumatic disorders (e.g., spinal cord disorders, head trauma, cerebrovascular disease, and stroke). Specifically, diseases associated with peripheral nerve injuries, peripheral neuropathy (e.g., resulting from chemotherapy or other medical therapies), localized neuropathies, and central nervous system diseases (e.g., Alzheimer's disease, Parkinson's disease, Huntington's disease, amyotrophic lateral sclerosis, and Shy-Drager syndrome), could all be treated using the polynucleotide or polypeptide of the present invention.

Chemotaxis

A polynucleotide or polypeptide of the present invention may have chemotaxis activity. A chemotaxic molecule attracts or mobilizes cells (e.g., monocytes, fibroblasts, neutrophils, T-cells, mast cells, eosinophils, epithelial and/or endothelial cells) to a particular site in the body, such as inflammation, infection, or site of hyperproliferation. The mobilized cells can then fight off and/or heal the particular trauma or abnormality.

A polynucleotide or polypeptide of the present invention may increase chemotaxic activity of particular cells. These chemotactic molecules can then be used to treat inflammation, infection, hyperproliferative disorders, or any immune system disorder by increasing the number of cells targeted to a particular location in the body. For example, chemotaxic molecules can be used to treat wounds and other trauma to tissues by attracting immune cells to the injured location. Chemotactic molecules of the present invention can also attract fibroblasts, which can be used to treat wounds.

It is also contemplated that a polynucleotide or polypeptide of the present invention may inhibit chemotactic activity. Such molecules could also be used to treat a variety of disorders. Thus, a polynucleotide or polypeptide of the present invention could be used as an inhibitor of chemotaxis. In atherosclerosis, the initial recruitament and activation of macrophages to vascular endothelium lesions may depend on chemotactic signals. Blockade of this step may prevent development of atherosclerotic plaques.

Atherosclerotic plaque develops over several decades and involves inflammatory cell infiltration, smooth muscle cell proliferation, accumulation of extracellular matrix, fibrous cap formation, and angiogenesis. (Bayes-Genis et al. Circ. Res. 86:125-130 (2000)). Chemotaxis is involved in the early development of atherosclerosis. Cell populations migrate toward the inner part of the vascular wall and originate the neointima, which leads to the formation of an atherosclerotic plaque. For example, monocyte chemotaxis is induced by monocyte chemoattractant protein 1 (MCP-1), which is expressed early in the development of atherosclerosis in the injured arterial wall. (Furukawa et al. Circ. Res. 84:306-314 (1999); Han et al. J. Lipid Res. 40:1053 (1999)). Additionally, insulin-like growth factors (IGF) have been shown to promote macrophage chemotaxis and also stimulate vascular smooth muscle proliferation and migration to form the neointima. (Bayes-Genis et al.). Activated platelets and C-reactive protein are important in inducing a significant increase in MCP-1 and recruiting monocytes, respectively. (Gawaz et al. Atherosclerosis, 148:75-85 (2000)); Torzewski et al. Arterioscler. Vasc. Biol. 20:2094-2099 (2000)). Furthermore, vascular endothelial growth factor (VEGF) has been shown to be a critical regulator of angiogenesis that stimulates proliferation, migration and proteolytic activity of endothelial cells. VEGF is able to stimulate chemotaxis in monocytes and can enhance matrix metalloproteinase expression and accelerate smooth muscle cell migration. (Wang & Keiser, Circ. Res. 83:832-840 (1998)). Blockade of one or more of these chemotaxic activities may prevent development of atherosclerotic plaques.

Binding Activity

A polypeptide of the present invention may be used to screen for molecules that bind to the polypeptide or for molecules to which the polypeptide binds. The binding of the polypeptide and the molecule may activate (i.e., an agonist), increase, inhibit (i.e., an antagonist), or decrease activity of the polypeptide or the molecule bound. Examples of such molecules include antibodies, oligonucleotides, proteins (e.g., receptors), or small molecules.

Preferably, the molecule is closely related to the natural ligand of the polypeptide, e.g., a fragment of the ligand, or a natural substrate, a ligand, a structural or functional mimetic (see, Coligan et al., Current Protocols in Immunology 1(2), Chapter 5 (1991)). Similarly, the molecule can be closely related to the natural receptor to which the polypeptide binds or, at least, related to a fragment of the receptor capable of being bound by the polypeptide (e.g., an active site). In either case, the molecule can be rationally designed using known techniques.

Preferably, the screening for these molecules involves producing appropriate cells which express the polypeptide, either as a secreted protein or on the cell membrane. Preferred cells include cells from mammals, yeast, Drosophila, or E. coli. Cells expressing the polypeptide (or cell membrane containing the expressed polypeptide) are then preferably contacted with a test compound potentially containing the molecule to observe binding, stimulation, or inhibition of activity of either the polypeptide or the molecule.

The assay may simply test binding of a candidate compound to the polypeptide, wherein binding is detected by a label, or in an assay involving competition with a labeled competitor. Further, the assay may test whether the candidate compound results in a signal generated by binding to the polypeptide.

Alternatively, the assay can be carried out using cell-free preparations, polypeptide/molecule affixed to a solid support, chemical libraries, or natural product mixtures. The assay may also simply comprise the steps of mixing a candidate compound with a solution containing a polypeptide, measuring polypeptide/molecule activity or binding, and comparing the polypeptide/molecule activity or binding to a standard.

Preferably, an ELISA assay can measure polypeptide level or activity in a sample (e.g., biological sample) using a monoclonal or polyclonal antibody. The antibody can measure polypeptide level or activity by either binding, directly or indirectly, to the polypeptide or by competing with the polypeptide for a substrate.

All of these above assays can be used as diagnostic or prognostic markers. The molecules discovered using these assays can be used to treat disease or to bring about a particular result in a patient (e.g., blood vessel growth) by activating or inhibiting the polypeptide/molecule. Moreover, the assays can discover agents which may inhibit or enhance the production of the polypeptide from suitably manipulated cells or tissues. At present, many of the diagnostic tools are only able to identify risk factors for atherosclerosis (e.g., hyper-lipidemia), and do not indicate the presence of actively developing atherosclerotic plaques. New assays using markers generated from materials of the present invention may provide some specific indicators of active disease.

Therefore, the invention includes a method of identifying compounds which bind to a polypeptide of the invention comprising the steps of: (a) incubating a candidate binding compound with a polypeptide of the invention; and (b) determining if binding has occurred. Moreover, the invention includes a method of identifying agonists/antagonists comprising the steps of: (a) incubating a candidate compound with a polypeptide of the invention, (b) assaying a biological activity, and (c) determining if a biological activity of the polypeptide has been altered.

Other Activities

A polypeptide or polynucleotide of the present invention may also increase or decrease the differentiation or proliferation of embryonic stem cells from a lineage other than the above-described hemopoietic lineage.

A polypeptide or polynucleotide of the present invention may also be used to modulate mammalian characteristics, such as body height, weight, hair color, eye color, skin, percentage of adipose tissue, pigmentation, size, and shape (e.g., cosmetic surgery). Similarly, a polypeptide or polynucleotide of the present invention may be used to modulate mammalian metabolism affecting catabolism, anabolism, processing, utilization, and storage of energy.

A polypeptide or polynucleotide of the present invention may be used to change a mammal's mental state or physical state by influencing biorhythms, circadian rhythms, depression (including depressive disorders), tendency for violence, tolerance for pain, the response to opiates and opioids, tolerance to opiates and opioids, withdrawal from opiates and opioids, reproductive capabilities (preferably by activin or inhibin-like activity), hormonal or endocrine levels, appetite, libido, memory, stress, or other cognitive qualities.

A polypeptide or polynucleotide of the present invention may also be used as a food additive or preservative, such as to increase or decrease storage capabilities, fat content, lipid, protein, carbohydrate, vitamins, minerals, cofactors, or other nutritional components.

Other Preferred Embodiments

Where a polynucleotide of the invention is down-regulated and exacerbates a pathological condition, such as atherosclerosis, the expression of the polynucleotide can be increased or the level of the intact polypeptide product can be increased in order to treat, prevent, ameliorate, or modulate the pathological condition. This can be accomplished by, for example, administering a polynucleotide or polypeptide of the invention to the mammalian subject.

A polynucleotide of the invention can be administered to a mammalian subject by a recombinant expression vector comprising the polynucleotide. A mammalian subject can be a human, baboon, chimpanzee, macaque, cow, horse, sheep, pig, horse, dog, cat, rabbit, guinea pig, rat or mouse. Preferably, the recombinant vector comprises a polynucleotide shown in SEQ ID NOs:1-55 or a polynucleotide which is at least 98% identical to a nucleic acid sequence shown in SEQ ID NOs:1-55. Also, preferably, the recombinant vector comprises a variant polynucleotide that is at least 80%, 90%, or 95% identical to a polynucleotide comprising SEQ ID NOs:1-55.

The administration of a polynucleotide or recombinant expression vector of the invention to a mammalian subject can be used to express a polynucleotide in said subject for the treatment of, for example, atherosclerosis. Expression of a polynucleotide in target cells, including but not limited to atherosclerosis cells, would effect greater production of the encoded polypeptide. In some cases where the encoded polypeptide is a nuclear protein, the regulation of other genes may be secondarily up- or down-regulated.

There are available to one skilled in the art multiple viral and non-viral methods suitable for introduction of a nucleic acid molecule into a target cell, as described above. In addition, a naked polynucleotide can be administered to target cells. Polynucleotides and recombinant expression vectors of the invention can be administered as a pharmaceutical composition. Such a composition comprises an effective amount of a polynucleotide or recombinant expression vector, and a pharmaceutically acceptable formulation agent selected for suitability with the mode of administration. Suitable formulation materials preferably are non-toxic to recipients at the concentrations employed and can modify, maintain, or preserve, for example, the pH, osmolarity, viscosity, clarity, color, isotonicity, odor, sterility, stability, rate of dissolution or release, adsorption, or penetration of the composition. See Remington 's Pharmaceutical Sciences (18^{th} Ed., A. R. Gennaro, ed., Mack Publishing Company 1990).

The pharmaceutically active compounds (i.e., a polynucleotide or a vector) can be processed in accordance with conventional methods of pharmacy to produce medicinal agents for administration to patients, including humans and other mammals. Thus, the pharmaceutical composition comprising a polynucleotide or a recombinant expression vector may be made up in a solid form (including granules, powders or suppositories) or in a liquid form (e.g., solutions, suspensions, or emulsions).

The dosage regimen for treating a disease with a composition comprising a polynucleotide or expression vector is based on a variety of factors, including the type or severity of the atherosclerosis, the age, weight, sex, medical condition of the patient, the route of administration, and the particular compound employed. Thus, the dosage regimen may vary widely, but can be determined routinely using standard methods. A typical dosage may range from about 0.1 mg/kg to about 100 mg/kg or more, depending on the factors mentioned above.

The frequency of dosing will depend upon the pharmacokinetic parameters of the polynucleotide or vector in the formulation being used. Typically, a clinician will administer the composition until a dosage is reached that achieves the desired effect. The composition may therefore be administered as a single dose, as two or more doses (which may or may not contain the same amount of the desired molecule) over time, or as a continuous infusion via implantation device or catheter. Further refinement of the appropriate dosage is routinely made by those of ordinary skill in the art and is within the ambit of tasks routinely performed by them. Appropriate dosages may be ascertained through use of appropriate dose-response data.

The cells of a mammalian subject may be transfected in vivo, ex vivo, or in vitro. Administration of a polynucleotide or a recombinant vector containing a polynucleotide to a target cell in vivo may be accomplished using any of a variety of techniques well known to those skilled in the art. For example, U.S. Pat. No. 5,672,344 describes an in vivo viral-mediated gene transfer system involving a recombinant neurotrophic HSV-1 vector. The above-described compositions of polynucleotides and recombinant vectors can be transfected in vivo by oral, buccal, parenteral, rectal, or topical administration as well as by inhalation spray. The term “parenteral” as used herein includes, subcutaneous, intravenous, intramuscular, intrastemal, infusion techniques or intraperitoneally.

While the nucleic acids and/or vectors of the invention can be administered as the sole active pharmaceutical agent, they can also be used in combination with one or more vectors of the invention or other agents. When administered as a combination, the therapeutic agents can be formulated as separate compositions that are given at the same time or different times, or the therapeutic agents can be given as a single composition.

Another delivery system for polynucleotides of the invention is a “non-viral” delivery system. Techniques that have been used or proposed for gene therapy include DNA-ligand complexes, adenovirus-ligand-DNA complexes, direct injection of DNA, CaPO₄ precipitation, gene gun techniques, electroporation, lipofection, and colloidal dispersion (Mulligan, R., (1993) Science, 260 (5110):926-32). Any of these methods are widely available to one skilled in the art and would be suitable for use in the present invention. Other suitable methods are available to one skilled in the art, and it is to be understood that the present invention may be accomplished using any of the available methods of transfection. Several such methodologies have been utilized by those skilled in the art with varying success (Mulligan, R., (1993) Science, 260 (5110):926-32).

Where a polynucleotide of the invention is up-regulated and exacerbates a pathological condition in a mammalian subject, such as atherosclerosis, the expression of the polynucleotide can be blocked or reduced or the level of the intact polypeptide product can be reduced in order to treat, prevent, ameliorate, or modulate the pathological condition. This can be accomplished by, for example, the use of antisense oligonucleotides or ribozymes. Alternatively, drugs or antibodies that bind to and inactivate the polypeptide product can be used.

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of gene products of the invention in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, (1994) Meth. Mol. Biol., 20:1-8; Sonveaux, (1994) Meth. Mol. Biol., 26:1-72; Uhlmann et al., (1990) Chem. Rev., 90:543-583.

Modifications of gene expression can be obtained by designing antisense oligonucleotides which will form duplexes to the control, 5′, or regulatory regions of a gene of the invention. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (e.g., Gee et al., in Huber & Carr, Molecular and Immunologic Approaches, Futura Publishing Co., Mt. Kisco, N.Y., 1994). An antisense oligonucleotide also can be designed to block translation of mRNA by preventing the transcript from binding to ribosomes.

Precise complementarity is not required for successful complex formation between an antisense oligonucleotide and the complementary sequence of a polynucleotide. Antisense oligonucleotides which comprise, for example, 2, 3, 4, or 5 or more stretches of contiguous nucleotides which are precisely complementary to a polynucleotide, each separated by a stretch of contiguous nucleotides which are not complementary to adjacent nucleotides, can provide sufficient targeting specificity for mRNA. Preferably, each stretch of complementary contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular polynucleotide sequence.

Antisense oligonucleotides can be modified without affecting their ability to hybridize to a polynucleotide of the invention. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′, 5′-substituted oligonucleotide in which the 3′ hydroxyl group or the 5′ phosphate group are substituted, also can be employed in a modified antisense oligonucleotide. These modified oligonucleotides can be prepared by methods well known in the art. See, e.g., Agrawal et al., (1992) Trends Biotechnol., 10:152-158; Uhlmann et al., (1990) Chem. Rev., 90:543-584; Uhlmann et al., (1987) Tetrahedron. Lett., 215:3539-3542.

Ribozymes are RNA molecules with catalytic activity. See, e.g., Cech, (1987) Science, 236:1532-1539; Cech, (1990) Ann. Rev. Biochem., 59:543-568; Cech, (1992) Curr. Opin. Struct. Biol., 2:605-609; Couture & Stinchcomb, (1996) Trends Genet., 12:510-515. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

The coding sequence of a polynucleotide of the invention can be used to generate ribozymes which will specifically bind to mRNA transcribed from the polynucleotide. Methods of designing and constructing ribozymes which can cleave RNA molecules in trans in a highly sequence specific manner have been developed and described in the art (see Haseloff et al. (1988) Nature, 334:585-591). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, e.g., Gerlach et al., EP 321,201).

Specific ribozyme cleavage sites within a RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate RNA targets also can be evaluated by testing accessibility to hybridization with complementary oligonucleotides using ribonuclease protection assays. The nucleotide sequences shown in SEQ ID NOs:1-55 and their complements provide sources of suitable hybridization region sequences. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease polynucleotide expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or UAS element, and a transcriptional terminator signal, for controlling transcription of ribozymes in the cells.

As taught in Haseloffet al., U.S. Pat. No. 5,641,673, ribozymes can be engineered so that ribozyme expression will occur in response to factors which induce expression of a target gene. Ribozymes also can be engineered to provide an additional level of regulation, so that destruction of mRNA occurs only when both a ribozyme and a target gene are induced in the cells.

Production of Diagnostic Tests

Pathological conditions or susceptibility to pathological conditions, such as atherosclerosis, can be diagnosed using methods of the invention. Testing for expression of a polynucleotide of the invention or for the presence of the polynucleotide product can correlate with the severity of the condition and can also indicate appropriate treatment. For example, the presence or absence of a mutation in a polynucleotide of the invention can be determined and a pathological condition or a susceptibility to a pathological condition is diagnosed based on the presence or absence of the mutation. Further, an alteration in expression of a polypeptide encoded by a polynucleotide of the invention can be detected, where the presence of an alteration in expression of the polypeptide is indicative of the pathological condition or susceptibility to the pathological condition. The alteration in expression can be an increase in the amount of expression or a decrease in the amount of expression.

As an additional method of diagnosis, a first biological sample from a patient suspected of having a pathological condition, such as atherosclerosis, is obtained along with a second sample from a suitable comparable control source. A biological sample can comprise saliva, blood, cerebrospinal fluid, amniotic fluid, urine, feces, or tissue, such as gastrointestinal tissue. A suitable control source can be obtained from one or more mammalian subjects that do not have the pathological condition. For example, the average concentrations and distribution of a polynucleotide or polypeptide of the invention can be determined from biological samples taken from a representative population of mammalian subjects, wherein the mammalian subjects are the same species as the subject from which the test sample was obtained. The amount of at least one polypeptide encoded by a polynucleotide of the invention is determined in the first and second sample. The amounts of the polypeptide in the first and second samples are compared. A patient is diagnosed as having a pathological condition if the amount of the polypeptide in the first sample is greater than or less than the amount of the polypeptide in the second sample. Preferably, the amount of polypeptide in the first sample falls in the range of samples taken from a representative group of patients with the pathological condition.

Other preferred embodiments of the claimed invention include an isolated nucleic acid molecule comprising a nucleotide sequence which is at least 80%, preferably at least 85%, more preferably at least 90%, most preferably at least 95% identical to a sequence of at least about 50 contiguous nucleotides in the nucleotide sequence of SEQ ID NOs:1-55.

Also preferred is a nucleic acid molecule wherein said sequence of contiguous nucleotides is included in the nucleotide sequence of SEQ ID NOs:1-55 in the range of positions beginning with the nucleotide at about the position of the 5′ nucleotide of the clone sequence and ending with the nucleotide at about the position of the 3′ nucleotide of the clone sequence.

Also preferred is a nucleic acid molecule wherein said sequence of contiguous nucleotides is included in the nucleotide sequence of SEQ ID NOs:1-55 in the range of positions beginning with the nucleotide at about the position of the 5′ nucleotide of the start codon and ending with the nucleotide at about the position of the 3′ nucleotide of the clone sequence as defined for SEQ ID NOs:1-55.

Similarly preferred is a nucleic acid molecule wherein said sequence of contiguous nucleotides is included in the nucleotide sequence of SEQ ID NOs:1-55 in the range of positions beginning with the nucleotide at about the position of the 5′ nucleotide of the first amino acid of the signal peptide and ending with the nucleotide at about the position of the 3′ nucleotide of the clone sequence as defined for SEQ ID NOs:1-55.

Also preferred is an isolated nucleic acid molecule comprising a nucleotide sequence which is at least 95% identical to a sequence of at least about 150 contiguous nucleotides in the nucleotide sequence of SEQ ID NOs:1-55.

Further preferred is an isolated nucleic acid molecule comprising a nucleotide sequence which is at least 95% identical to a sequence of at least about 500 contiguous nucleotides in the nucleotide sequence of SEQ ID NOs:1-55.

A further preferred embodiment is a nucleic acid molecule comprising a nucleotide sequence which is at least 95% identical to the nucleotide sequence of SEQ ID NOs:1-55 beginning with the nucleotide at about the position of the 5′ nucleotide of the first amino acid of the signal peptide and ending with the nucleotide at about the position of the 3′ nucleotide of the clone sequence as defined for SEQ ID NOs:1-55.

A further preferred embodiment is an isolated nucleic acid molecule comprising a nucleotide sequence which is at least 95% identical to the complete nucleotide sequence of SEQ ID NOs:1-55.

Also preferred is an isolated nucleic acid molecule which hybridizes under stringent hybridization conditions to a nucleic acid molecule, wherein said nucleic acid molecule which hybridizes does not hybridize under stringent hybridization conditions to a nucleic acid molecule having a nucleotide sequence consisting of only A residues or of only T residues.

A further preferred embodiment is a method for detecting in a biological sample a nucleic acid molecule comprising a nucleotide sequence which is at least 95% identical to a sequence of at least 35 contiguous nucleotides in a sequence selected from the group consisting of a nucleotide sequence of SEQ ID NOs:1-55, which method comprises a step of comparing a nucleotide sequence of at least one nucleic acid molecule in said sample with a sequence selected from said group and determining whether the sequence of said nucleic acid molecule in said sample is at least 95% identical to said selected sequence.

Also preferred is the above method wherein said step of comparing sequences comprises determining the extent of nucleic acid hybridization between nucleic acid molecules in said sample and a nucleic acid molecule comprising said sequence selected from said group. Similarly, also preferred is the above method wherein said step of comparing sequences is performed by comparing the nucleotide sequence determined from a nucleic acid molecule in said sample with said sequence selected from said group. The nucleic acid molecules can comprise DNA molecules or RNA molecules.

A further preferred embodiment is a method for identifying the species, tissue or cell type of a biological sample, which method comprises a step of detecting nucleic acid molecules in said sample, if any, comprising a nucleotide sequence that is at least 95% identical to a sequence of at least 35 contiguous nucleotides in a sequence selected from the group consisting of a nucleotide sequence of SEQ ID NOs:1-55.

Also preferred is a method for diagnosing in a subject a pathological condition associated with abnormal structure or expression of a gene, which method comprises a step of detecting in a biological sample obtained from said subject nucleic acid molecules, if any, comprising a nucleotide sequence that is at least 95% identical to a sequence of at least 35 contiguous nucleotides in a sequence selected from the group consisting of a nucleotide sequence of SEQ ID NOs:1-55.

The method for diagnosing a pathological condition can comprise a step of detecting nucleic acid molecules comprising a nucleotide sequence in a panel of at least two nucleotide sequences, wherein at least one sequence in said panel is at least 95% identical to a sequence of at least 35 contiguous nucleotides in a sequence selected from said group.

Also preferred is a composition of matter comprising isolated nucleic acid molecules wherein the nucleotide sequences of said nucleic acid molecules comprise a panel of at least two nucleotide sequences, wherein at least one sequence in said panel is at least 95% identical to a sequence of at least 35 contiguous nucleotides in a sequence selected from the group consisting of a nucleotide sequence of SEQ ID NOs:1-55. The nucleic acid molecules can comprise DNA molecules or RNA molecules.

Also preferred is an isolated polypeptide comprising an amino acid sequence at least 90% identical to a sequence of at least about 10 contiguous amino acids in an amino acid sequence translated from SEQ ID NOs:1-55.

Also preferred is a polypeptide, wherein said sequence of contiguous amino acids is included in amino acids in an amino acid sequence translated from SEQ ID NOs:1-55, in the range of positions beginning with the residue at about the position of the first amino acid of the secreted portion and ending with the residue at about the last amino acid of the open reading frame.

Also preferred is an isolated polypeptide comprising an amino acid sequence at least 95% identical to a sequence of at least about 30 contiguous amino acids in an amino acid sequence translated from SEQ ID NOs:1-55.

Further preferred is an isolated polypeptide comprising an amino acid sequence at least 95% identical to a sequence of at least about 100 contiguous amino acids in an amino acid sequence translated from SEQ ID NOs:1-55.

Further preferred is an isolated polypeptide comprising an amino acid sequence at least 95% identical to amino acids in an amino acid sequence translated from SEQ ID NOs:1-55.

Further preferred is a method for detecting in a biological sample a polypeptide comprising an amino acid sequence which is at least 90% identical to a sequence of at least 10 contiguous amino acids in a sequence selected from the group consisting of amino acid sequences translated from SEQ ID NOs:1-55, which method comprises a step of comparing an amino acid sequence of at least one polypeptide molecule in said sample with a sequence selected from said group and determining whether the sequence of said polypeptide molecule in said sample is at least 90% identical to said sequence of at least 10 contiguous amino acids.

Also preferred is the above method wherein said step of comparing an amino acid sequence of at least one polypeptide molecule in said sample with a sequence selected from said group comprises determining the extent of specific binding of polypeptides in said sample to an antibody which binds specifically to a polypeptide comprising an amino acid sequence that is at least 90% identical to a sequence of at least 10 contiguous amino acids in a sequence selected from the group consisting of amino acid sequences translated from SEQ ID NOs:1-55.

Also preferred is the above method wherein said step of comparing sequences is performed by comparing the amino acid sequence determined from a polypeptide molecule in said sample with said sequence selected from said group.

Also preferred is a method for identifying the species, tissue or cell type of a biological sample, which method comprises a step of detecting polypeptide molecules in said sample, if any, comprising an amino acid sequence that is at least 90% identical to a sequence of at least 10 contiguous amino acids in a sequence selected from the group consisting of amino acid sequences translated from SEQ ID NOs:1-55.

Also preferred is the above method for identifying the species, tissue or cell type of a biological sample, which method comprises a step of detecting polypeptide molecules comprising an amino acid sequence in a panel of at least two amino acid sequences, wherein at least one sequence in said panel is at least 90% identical to a sequence of at least 10 contiguous amino acids in a sequence selected from the above group.

Also preferred is a method for diagnosing in a subject a pathological condition associated with abnormal structure or expression of a gene, which method comprises a step of detecting in a biological sample obtained from said subject polypeptide molecules comprising an amino acid sequence in a panel of at least two amino acid sequences, wherein at least one sequence in said panel is at least 90% identical to a sequence of at least 10 contiguous amino acids in a sequence selected from the group consisting of amino acid sequences translated from SEQ ID NOs:1-55.

In any of these methods, the step of detecting said polypeptide molecules includes using an antibody.

Also preferred is an isolated nucleic acid molecule comprising a nucleotide sequence which is at least 95% identical to a nucleotide sequence encoding a polypeptide wherein said polypeptide comprises an amino acid sequence that is at least 90% identical to a sequence of at least 10 contiguous amino acids in a sequence selected from the group consisting of amino acid sequences translated from SEQ ID NOs:1-55.

Also preferred is an isolated nucleic acid molecule, wherein said nucleotide sequence encoding a polypeptide has been optimized for expression of said polypeptide in a prokaryotic host.

Also preferred is an isolated nucleic acid molecule, wherein said nucleotide sequence encodes a polypeptide comprising an amino acid sequence selected from the group consisting of amino acid sequences translated from SEQ ID NOs:1-55.

Further preferred is a method of making a recombinant vector comprising inserting any of the above isolated nucleic acid molecule into a vector. Also preferred is the recombinant vector produced by this method. Also preferred is a method of making a recombinant host cell comprising introducing the vector into a host cell, as well as the recombinant host cell produced by this method.

Also preferred is a method of making an isolated polypeptide comprising culturing this recombinant host cell under conditions such that said polypeptide is expressed and recovering said polypeptide. Also preferred is this method of making an isolated polypeptide, wherein said recombinant host cell is a eukaryotic cell and said polypeptide is a secreted portion of a human secreted protein comprising an amino acid sequence selected from the group consisting of amino acid sequences translated from SEQ ID NOs:1-55. The isolated polypeptide produced by this method is also preferred.

Also preferred is a method of treatment of an individual in need of an increased level of a secreted protein activity, which method comprises administering to such an individual a pharmaceutical composition comprising an amount of an isolated polypeptide, polynucleotide, or antibody of the claimed invention effective to increase the level of said protein activity in said individual.

The present invention also includes a diagnostic system, preferably in kit form, for assaying for the presence of the polypeptide of the present invention in a body sample, such as brain tissue, cell suspensions or tissue sections; or a body fluid sample, such as CSF, blood, plasma or serum, where it is desirable to detect the presence, and preferably the amount, of the polypeptide of this invention in the sample according to the diagnostic methods described herein.

In a related embodiment, a nucleic acid molecule can be used as a probe (i.e., an oligonucleotide) to detect the presence of a polynucleotide of the present invention, a gene corresponding to a polynucleotide of the present invention, or a mRNA in a cell that is diagnostic for the presence or expression of a polypeptide of the present invention in the cell. The nucleic acid molecule probes can be of a variety of lengths from at least about 10, suitably about 10 to about 5000 nucleotides long, although they will typically be about 20 to 500 nucleotides in length. Hybridization methods are extremely well known in the art and will not be described further here.

In a related embodiment, detection of genes corresponding to the polynucleotides of the present invention can be conducted by primer extension reactions such as the polymerase chain reaction (PCR). To that end, PCR primers are utilized in pairs, as is well known, based on the nucleotide sequence of the gene to be detected. Preferably, the nucleotide sequence is a portion of the nucleotide sequence of a polynucleotide of the present invention. Particularly preferred PCR primers can be derived from any portion of a DNA sequence encoding a polypeptide of the present invention, but are preferentially from regions which are not conserved in other cellular proteins.

Preferred PCR primer pairs useful for detecting the genes corresponding to the polynucleotides of the present invention and expression of these genes are described in the Examples, including the corresponding Tables. Nucleotide primers from the corresponding region of the polypeptides of the present invention described herein are readily prepared and used as PCR primers for detection of the presence or expression of the corresponding gene in any of a variety of tissues.

The diagnostic system includes, in an amount sufficient to perform at least one assay, a subject polypeptide of the present invention, a subject antibody or monoclonal antibody, and/or a subject nucleic acid molecule probe of the present invention, as a separately packaged reagent.

In another embodiment, a diagnostic system, preferably in kit form, is contemplated for assaying for the presence of the polypeptide of the present invention or an antibody immunoreactive with the polypeptide of the present invention in a body fluid sample. Such diagnostic kit would be useful for monitoring the fate of a therapeutically administered polypeptide of the present invention or an antibody immunoreactive with the polypeptide of the present invention. The system includes, in an amount sufficient for at least one assay, a polypeptide of the present invention and/or a subject antibody as a separately packaged immunochemical reagent.

Instructions for use of the packaged reagent(s) are also typically included.

As used herein, the term “package” refers to a solid matrix or material such as glass, plastic (e.g., polyethylene, polypropylene, or polycarbonate), paper, foil and the like capable of holding within fixed limits a polypeptide, polyclonal antibody, or monoclonal antibody of the present invention. Thus, for example, a package can be a glass vial used to contain milligram quantities of a contemplated polypeptide or antibody or it can be a microtiter plate well to which microgram quantities of a contemplated polypeptide or antibody have been operatively affixed (i.e., linked) so as to be capable of being immunologically bound by an antibody or antigen, respectively.

“Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like.

A diagnostic system of the present invention preferably also includes a label or indicating means capable of signaling the formation of an immunocomplex containing a polypeptide or antibody molecule of the present invention.

The word “complex” as used herein refers to the product of a specific binding reaction such as an antibody-antigen or receptor-ligand reaction. Exemplary complexes are immunoreaction products.

As used herein, the terms “label” and “indicating means” in their various grammatical forms refer to single atoms and molecules that are either directly or indirectly involved in the production of a detectable signal to indicate the presence of a complex. Any label or indicating means can be linked to or incorporated in an expressed protein, polypeptide, or antibody molecule that is part of an antibody or monoclonal antibody composition of the present invention or used separately, and those atoms or molecules can be used alone or in conjunction with additional reagents. Such labels are themselves well-known in clinical diagnostic chemistry and constitute a part of this invention only insofar as they are utilized with otherwise novel proteins methods and/or systems.

The labeling means can be a fluorescent labeling agent that chemically binds to antibodies or antigens without denaturing them to form a fluorochrome (dye) that is a useful immunofluorescent tracer. Suitable fluorescent labeling agents are fluorochromes such as fluorescein isocyanate (FIC), fluorescein isothiocyanate (FITC), 5-dimethylamine-1-naphthalenesulfonyl chloride (DANSC), tetramethylrhodamine isothiocyanate (TRITC), lissamine, rhodamine 8200 sulphonyl chloride (RB 200 SC) and the like. A description of immunofluorescence analysis techniques is found in DeLuca, “Immunofluoreseence Analysis”, in Antibody As a Tool, Marchalonis et al., Eds., John Wiley & Sons, Ltd., pp. 189-231 (1982), which is incorporated herein by reference. Other suitable labeling agents are known to those skilled in the art.

In preferred embodiments, the indicating group is an enzyme, such as horseradish peroxidase (HRP), glucose oxidase, or the like. In such cases where the principal indicating group is an enzyme such as HRP or glucose oxidase, additional reagents are required to visualize the fact that a receptor-ligand complex (immunoreactant) has formed. Such additional reagents for HRP include hydrogen peroxide and an oxidation dye precursor such as diaminobenzidine. An additional reagent useful with glucose oxidase is 2,2′-amino-di-(3-ethyl-benzthiazoline-G-sulfonic acid) (ABTS).

Radioactive elements are also useful labeling agents and are used illustratively herein. An exemplary radiolabeling agent is a radioactive element that produces gamma ray emissions. Elements which themselves emit gamma rays, such as ¹²⁴I, ¹²⁵I, ¹³²I and ⁵¹Cr represent one class of gamma ray emission-producing radioactive element indicating groups. Particularly preferred is ¹²⁵I. Another group of useful labeling means are those elements such as ¹¹C, ¹⁸F, ¹⁵O and ¹³N which themselves emit positrons. The positrons so emitted produce gamma rays upon encounters with electrons present in the animal's body. Also useful is a beta emitter, such ¹¹¹indium or ³H.

The linking of labels or labeling of polypeptides and proteins is well known in the art. For instance, antibody molecules produced by a hybridoma can be labeled by metabolic incorporation of radioisotope-containing amino acids provided as a component in the culture medium (see, e.g., Galfre et al., Meth. Enzymol., 73:3-46 (1981)). The techniques of protein conjugation or coupling through activated functional groups are particularly applicable (see, e.g., Aurameas, et al., Scand. J. Immunol., Vol. 8 Suppl. 7:7-23 (1978); Rodwell et al., Biotech., 3:889-894 (1984); and U.S. Pat. No. 4,493,795).

The diagnostic systems can also include, preferably as a separate package, a specific binding agent. A “specific binding agent” is a molecular entity capable of selectively binding a reagent species of the present invention or a complex containing such a species, but is not itself a polypeptide or antibody molecule composition of the present invention. Exemplary specific binding agents are second antibody molecules, complement proteins or fragments thereof, S. aureus protein A, and the like. Preferably the specific binding agent binds the reagent species when that species is present as part of a complex.

In preferred embodiments, the specific binding agent is labeled. However, when the diagnostic system includes a specific binding agent that is not labeled, the agent is typically used as an amplifying means or reagent. In these embodiments, the labeled specific binding agent is capable of specifically binding the amplifying means when the amplifying means is bound to a reagent species-containing complex.

The diagnostic kits of the present invention can be used in an “ELISA” format to detect the quantity of the polypeptide of the present invention in a sample. “ELISA” refers to an enzyme-linked immunosorbent assay that employs an antibody or antigen bound to a solid phase and an enzyme-antigen or enzyme-antibody conjugate to detect and quantify the amount of an antigen present in a sample. A description of the ELISA technique is found in Sites et al., Basic and Clinical Immunology, 4^{th} Ed., Chap. 22, Lange Medical Publications, Los Altos, Calif. (1982) and in U.S. Pat. No. 3,654,090; U.S. Pat. No. 3,850,752; and U.S. Pat. No. 4,016,043, which are all incorporated herein by reference.

Thus, in some embodiments, a polypeptide of the present invention, an antibody or a monoclonal antibody of the present invention can be affixed to a solid matrix to form a solid support that comprises a package in the subject diagnostic systems.

A reagent is typically affixed to a solid matrix by adsorption from an aqueous medium, although other modes of affixation applicable to proteins and polypeptides can be used that are well known to those skilled in the art. Exemplary adsorption methods are described herein.

Useful solid matrices are also well known in the art. Such materials are water insoluble and include the cross-linked dextran available under the trademark SEPHADEX from Pharmacia Fine Chemicals (Piscataway, N.J.), agarose, polystyrene beads of about 1 micron (μm) to about 5 millimeters (mm) in diameter available from several suppliers (e.g., Abbott Laboratories, Chicago, Ill.), polyvinyl chloride, polystyrene, cross-linked polyacrylamide, nitrocellulose- or nylon-based webs (sheets, strips or paddles) or tubes, plates or the wells of a microtiter plate, such as those made from polystyrene or polyvinylchloride.

The reagent species, labeled specific binding agent, or amplifying reagent of any diagnostic system described herein can be provided in solution, as a liquid dispersion or as a substantially dry power, e.g., in lyophilized form. Where the indicating means is an enzyme, the enzyme's substrate can also be provided in a separate package of a system. A solid support such as the before-described microtiter plate and one or more buffers can also be included as separately packaged elements in this diagnostic assay system.

The packaging materials discussed herein in relation to diagnostic systems are those customarily utilized in diagnostic systems.

Having generally described the invention, the same will be more readily understood by reference to the following examples, which are provided by way of illustration and are not intended as limiting.

### EXAMPLE 1

### Identification and Characterization of Polynucleotides Up-Regulated by Fatty Lesion Development

Methods

Studies were designed to identify aorta transcripts that are regulated by fatty lesion development caused by a high cholesterol diet and also to identify aorta transcripts responsive to lercanidipine treatment. The TOGA (Total Gene Analysis) method was used to identify digital sequence tags (DSTs) corresponding to mRNAs which expression is regulated by fatty lesion development caused by hypercholesterolemia, regulated by lercanidipine treatment, or regulated by hypercholesterolemia and reversed by lercanidipine treatment. In addition, mRNAs which expression is differentially regulated by lercanidipine racemate and the (R)-enantiomer of lercanidipine were identified.

To perform the studies, New Zealand male rabbits weighing 2.0-2.5 kg (Charles River, Calco, Italy) were used. The rabbits were divided into three groups and maintained in identical experimental conditions: (1) control group (n=15); rabbits fed a cholesterol-rich diet; (2) lercanidipine-treated group (n=15); rabbits fed a cholesterol-rich diet and also treated with lercanidipine (3 mg/kg/week); and (3) (R)-lercanidipine-treated group (n=5); rabbits fed a cholesterol-rich diet and also treated with the (R)-enantiomer of lercanidipine (3 mg/kg/week). After a 2 week period of subcutaneous, once a week, pretreatment with 3 mg/kg/week of lercanidipine or (R)-lercanidipine, the rabbits received a daily cholesterol supplement for up to 8 weeks or 2 weeks, respectively. In the following examples, use of the term “lercanidipine” refers to the racemic mixture of lercanidipine hydrochloride, whereas “(R)-lercanidipine” refers to the (R)-enantiomer of lercanidipine hydrochloride.

The mRNA from the above-described groups was isolated from aorta at different time points, resulting in the following mRNA samples: (1) Day 0 Control=no administered cholesterol, no lercanidipine; (2) Day 0+lercanidipine=no administered cholesterol, treatment with lercanipidine (2 weeks; 3 mg/kg/week); (3) Day 14 Control=cholesterol diet for two weeks, no lercanidipine; (4) Day 14+lercanidipine=cholesterol diet for two weeks, treatment with lercanidipine (four weeks; 3 mg/kg/week); (5) Week 8 Control=cholesterol diet for 8 weeks, no lercanidipine; and (6) Week 8+lercanidipine=cholesterol diet for 8 weeks, treatment with lercanidipine (10 weeks; 3 mg/kg/week). In addition, Day 14+(R)-lercanidipine mRNA samples were prepared from rabbits given a cholesterol diet for two weeks and treated with 3 mg/kg/week (R)-lercanidipine for four weeks.

The daily doses of cholesterol (1.6 g) were given in the morning (at 08.00 hours) each mixed in 20 g of food pellets. Normal chow, up to 150 g, was added after all the cholesterol-rich diet was eaten (usually within 30 minutes). The hydrochloride salt of lercanidipine or (R)-lercanidipine (Recordati, Milano, Italy) was administered subcutaneously as a solution in 50% propylene glycol. The doses of lercanidipine utilized here did not affect arterial blood pressure. Animals had free access to water and were kept in a 12 hour light-dark cycle. Blood was drawn from the central ear artery at day 29 after treatment, started in order to monitor the lercanidipine plasma level at 24 hour post-dose.

The doses of lercanidipine used were determined from preliminary kinetic studies. In rabbits, the subcutaneous administration of 3 and 1 mg/kg lercanidipine resulted in plasma levels of 3.2 and 0.5 mg/kg respectively, after 7 days from the administration.

Total serum cholesterol was measured at sacrifice by the enzymatic procedure described in Catapano et al., Ann. N.Y. Acad. Sci., 522:519-521 (1988). High density lipoprotein (HDL) cholesterol was determined by the same method after very low density and low density lipoproteins (LDL) precipitation with phosphotungstic acid (Catapano et al. (1988), supra). At the end of the treatment, the animals were sacrificed by an overdose of sodium pentobarbital (65 mg/kg) administered intravenously.

Fatty lesion formation caused by the high-cholesterol diet was evidenced by staining aortic lipids. The aortas were retrieved after sacrifice, cleaned from blood and adherent tissue, and fixed in buffered formaldehyde (10%) for 24 hours at 4° C. Aortic lipids were stained with Sudan IV according to the method described in (Catapano et al. (1988), supra). The extension of aortic atherosclerotic plaques, determined by Sudan IV stainable areas, was measured by planimetry and expressed as percent of aorta inner surface covered by plaques.

Aorta mRNAs were prepared in the following manner. First, the aortas from each experimental group were retrieved and shredded using a polytron homogenizer. The samples were further homogenized using a teflon pestle, after which the cellular debris, nuclei, and blood cells were pelleted by centrifugation. The supernatants were extracted twice with phenol-chloroform-isoamyl alcohol and once with chloroform-isoamyl alcohol. RNA was then precipitated from the aqueous phase with ethanol. The poly A+ mRNA was prepared using standard methods of polyA selection known in the art (Schriber et al., J. Mol. Biol., 142:93-116 (1980)).

The isolated mRNA was analyzed using a method of simultaneous sequence-specific identification of mRNAs known as TOGA, described in U.S. Pat. No. 5,459,037 and U.S. Pat. No. 5,807,680, hereby incorporated herein by reference. In a preferred embodiment, the TOGA method further comprised an additional PCR step performed using a mixture of four 5′ PCR primers and cDNA templates prepared from a population of antisense cRNAs. A final PCR step that used a mixture of 256 5′ PCR primers produced PCR products that were cDNA fragments that corresponded to the 3′-region of the starting mRNA population. The produced PCR products were then identified by: (a) the initial 5′ sequence comprising the sequence remainder of the recognition site of the restriction endonuclease used to cut and isolate the 3′ region plus the sequence of the preferably four parsing bases immediately 3′ to the remainder of the recognition site, preferably the sequence of the entire fragment; and (b) the length of the fragment. These two parameters, sequence and fragment length, were used to compare the obtained PCR products to a database of known polynucleotide sequences. Since the length of the obtained PCR products includes known vector sequences at the 5′ and 3′ ends of the insert, the sequence of the insert provided in the sequence listing is shorter than the fragment length that forms part of the digital address.

The method yields Digital Sequence Tags (DSTs), that is, polynucleotides that are expressed sequence tags of the 3′ end of mRNAs. DSTs that showed changes in relative levels during fatty lesion development or as a result of lercanidipine treatment were selected for further study. The intensities of the laser-induced fluorescence of the labeled PCR products were compared across aortic sample isolated from control (no lercanidipine) or lercanidipine-treated rabbits at day 0, Day 14, and Week 8 of cholesterol treatment.

In general, double-stranded cDNA is generated from poly(A)-enriched cytoplasmic RNA extracted from the tissue samples of interest using an equimolar mixture of all 48 5′-biotinylated anchor primers of a set to initiate reverse transcription. One such suitable set is G-A-A-T-T-C-A-A-C-T-G-G-A-A-G-C-G-G-C-C-G-C-A-G-G-A-A-T-T-T-T-T-T-T-T-T-T-T-T-T-T-T-T-T-T-V-N-N (SEQ ID NO: 56), where V is A, C or G and N is A, C, G or T. One member of this mixture of 48 anchor primers initiates synthesis at a fixed position at the 3′ end of all copies of each mRNA species in the sample, thereby defining a 3′ endpoint for each species, resulting in biotinylated double-stranded cDNA.

Each biotinylated double-stranded cDNA sample was cleaved with the restriction endonuclease MspI, which recognizes the sequence CCGG. The resulting fragments of cDNA corresponding to the 3′ region of the starting mRNA were then isolated by capture of the biotinylated cDNA fragments on a streptavidin-coated substrate. Suitable streptavidin-coated substrates include microtitre plates, PCR tubes, polystyrene beads, paramagnetic polymer beads and paramagnetic porous glass particles. A preferred streptavidin-coated substrate is a suspension of paramagnetic polymer beads (Dynal, Inc., Lake Success, N.Y.).

After washing the streptavidin-coated substrate and captured biotinylated cDNA fragments, the cDNA fragment product was released by digestion with NotI, which cleaves at an 8-nucleotide sequence within the anchor primers but rarely within the mRNA-derived portion of the cDNAs. The MspI-NotI fragments of cDNA corresponding to the 3′ region of the starting mRNA, which are of uniform length for each mRNA species, were directionally ligated into ClaI-NotI-cleaved plasmid pBC SK⁺ (Stratagene, La Jolla, Calif.) in an antisense orientation with respect to the vector's T3 promoter, and the product used to transform Escherichia coli SURE cells (Stratagene). The ligation regenerates the NotI site, but not the MspI site, leaving CGG as the first 3 bases of the 5′ end of all PCR products obtained. Each library contained in excess of 5×10⁵ recombinants to ensure a high likelihood that the 3′ ends of all mRNAs with concentrations of 0.001% or greater were multiply represented. Plasmid preps (Qiagen) were made from the cDNA library of each sample under study.

An aliquot of each library was digested with MspI, which effects linearization by cleavage at several sites within the parent vector while leaving the 3′ cDNA inserts and their flanking sequences, including the T3 promoter, intact. The product was incubated with T3 RNA polymerase (MEGAscript kit, Ambion) to generate antisense cRNA transcripts of the cloned inserts containing known vector sequences abutting the MspI and NotI sites from the original cDNAs.

At this stage, each of the cRNA preparations was processed in a three-step fashion. In step one, 250 ng of cRNA was converted to first-strand cDNA using the 5′ RT primer (A-G-G-T-C-G-A-C-G-G-T-A-T-C-G-G, (SEQ ID NO: 57). In step two, 400 pg of cDNA product was used as PCR template in four separate reactions with each of the four 5′ PCR primers of the form G-G-T-C-G-A-C-G-G-T-A-T-C-G-G-N (SEQ ID NO: 58), each paired with a “universal” 3′ PCR primer G-A-G-C-T-C-C-A-C-C-G-C-G-G-T (SEQ ID NO: 59).

In step three, the product of each subpool was further divided into 64 subsubpools (2 ng in 20 μl) for the second PCR reaction, with 100 ng each of the fluoresceinated “universal” 3′ PCR primer, the oligonucleotide (SEQ ID NO: 59) conjugated to 6-FAM and the appropriate 5′ PCR primer of the form C-G-A-C-G-G-T-A-T-C-G-G-N-N-N-N (SEQ ID NO: 60), using a program that included an annealing step at a temperature X slightly above the Tm of each 5′ PCR primer to minimize artifactual mispriming and promote high fidelity copying. Each polymerase chain reaction step was performed in the presence of TaqStart antibody (Clontech).

The products from the final polymerase chain reaction step for each of the tissue samples were resolved on a series of denaturing DNA sequencing gels using the automated ABI Prizm 377 sequencer. Data were collected using the GeneScan software package (ABI) and normalized for amplitude and migration. Complete execution of this series of reactions generated 64 product subpools for each of the four pools established by the 5′ PCR primers of the first PCR reaction, for a total of 256 product subpools for the entire 5′ PCR primer set of the second PCR reaction.

The mRNA samples from rabbit aorta prepared as described above were analyzed. Table 1 is a summary of the expression levels of 594 mRNAs determined from cDNA. These cDNA molecules are identified by their digital address, that is, a partial 5′ terminus nucleotide sequence comprising the remainder of the MspI site and the four parsing bases for the 5′ PCR primer of each subsubpool coupled with the length of the molecule, as well as the relative amount of the molecule produced in control and lercanidipine-treated animals at different time intervals after cholesterol treatment. The 5′ terminus partial nucleotide sequence is determined by the recognition site for MspI and the nucleotide sequence of the parsing bases of the 5′ PCR primer used in the final PCR step. The length of the fragment was determined by interpolation on a standard curve.

For example, the entry in Table 1 that describes a DNA molecule identified by the digital address MspI AAGC 288 is further characterized as having a 5′ terminus partial nucleotide sequence of CGGAAGC and a digital address length of 288 b.p. The DNA molecule identified as MspI AAGC 288 is further described as being expressed at low levels in normal or control aorta at day 0 (no cholesterol, no lercanidipine) (251) and lercanidipine-treated aorta at day (no cholesterol, 3 mg/kg/week lercanidipine for 2 weeks) (215). The same DNA molecule is expressed at increasing levels after 14 days of cholesterol treatment (1008), which increase may be unaffected or slightly decreased by lercanidipine treatment (753) or R-lercanipidine treatment (872). The expression of this molecule is even greater at 8 weeks of cholesterol administration (1673) and does not appear to be affected by lercanidipine treatment (1733). Thus, a DNA molecule whose expression is up-regulated in a sustained manner by fatty lesion development induced by hypercholesterolemia has been identified. Additionally, the DNA molecule identified as MspI AAGC 288 (REC1_{—}1) is described by its nucleotide sequence which corresponds with SEQ ID NO: 1.

Similarly, the other DNA molecules identified in Table 1 by their MspI digital addresses are further characterized by: (1) the level of gene expression in control rabbit aorta (no cholesterol, no lercanidipine); (2) the level of gene expression in aorta of lercanidipine-treated rabbits (no cholesterol, 3 mg/kg/week lercanidipine for 2 weeks); (3) the level of gene expression in control rabbit aorta at day 14 (14 day cholesterol diet, no lercanidipine); (4) the level of gene expression in aorta of lercanidine-treated rabbits at day 14 (14 day cholesterol diet, 3 mg/kg/week lercanidipine for 4 weeks); (5) the level of gene expression in aorta of (R)-lercanidipine-treated rabbits at day 14 (14 day cholesterol diet, 3 mg/kg/week (R)-lercanidipine for 4 weeks); (6) the level of gene expression in control rabbit aorta at week 8 (8 week cholesterol diet, no lercanidipine); and (7) the level of gene expression in aorta of lercanidine-treated rabbits at week 8 (8 week cholesterol diet, 3 mg/kg/week lercanidipine for 10 weeks).

Additionally, several of the isolated clones were further characterized as shown in Tables 2-4, and their nucleotide sequences are provided as SEQ ID NO: 1-55 in the Sequence Listing below.

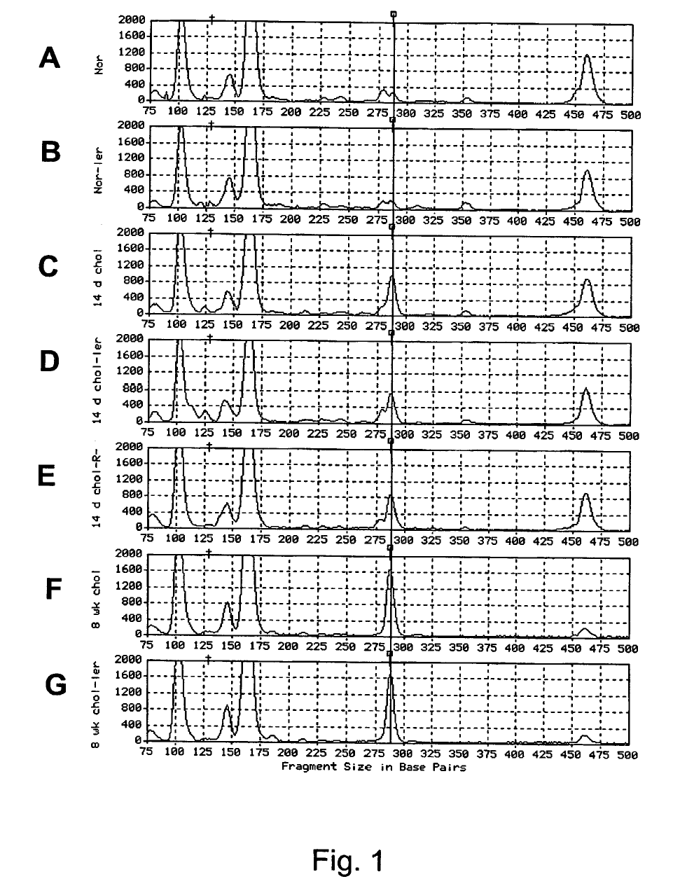

The data shown in FIG. 1 were generated with a 5′-PCR primer (C-G-A-C-G-G-T-A-T-C-G-G-A-A-G-C, SEQ ID NO: 61) paired with the “universal” 3′ primer (SEQ ID NO: 59) labeled with 6-carboxyfluorescein (6FAM, ABI) at the 5′ terminus. PCR reaction products were resolved by gel electrophoresis on 4.5% acrylamide gels and fluorescence data acquired on ABI377 automated sequencers. Data were analyzed using GeneScan software (Perkin-Elmer).

The results of TOGA analysis using a 5′ PCR primer with parsing bases AAGC (SEQ ID NO: 61) are shown in FIG. 1, which shows the PCR products produced from mRNA extracted from (A) normal control aorta (no cholesterol, no lercanidipine); (B) normal lercanidipine-treated aorta (no cholesterol, 3 mg/kg/week lercanidipine for 2 weeks); (C) control aorta at day 14 of cholesterol administration (no lercanidipine); (D) lercanidipine-treated aorta at day 14 of cholesterol administration (3 mg/kg/week lercanidipine for 4 weeks); (E) (R)-lercanidipine-treated aorta at day 14 of cholesterol administration (3 mg/kg/week (R)-lercanidipine for 4 weeks); (F) control aorta at week 8 of cholesterol administration (no lercanidipine); and (G) lercanidipine-treated aorta at week 8 of cholesterol administration (3 mg/kg/week lercanidipine for 10 weeks). The vertical index line indicates a PCR product of about 288 b.p. that shows greater expression in the aorta of rabbits fed a high cholesterol diet than those fed a normal diet.

In other cases, the TOGA PCR product was sequenced using a modification of a direct sequencing methodology (Innis et al., Proc. Nat'l. Acad. Sci., 85: 9436-9440 (1988)). PCR products corresponding to DSTs were gel purified and PCR amplified again to incorporate sequencing primers at the 5′- and 3′- ends. The sequence addition was accomplished through 5′ and 3′ ds-primers containing M13 sequencing primer sequences (M13 forward and M13 reverse respectively) at their 5′ ends, followed by a linker sequence and a sequence complementary to the DST ends. Using the Clontech Taq Start antibody system, a master mix containing all components except the gel purified PCR product template was prepared, which contained sterile H₂O, 10×PCR II buffer, 10 mM dNTP, 25 mM MgCl₂, AmpliTaq/Antibody mix (1.1 μg/μl Taq antibody, 5 U/μl AmpliTaq), 100 ng/μl of 5′ ds-primer (5′ TCC CAG TCA CGA CGT TGT AAA ACG ACG GCT CAT ATG AAT TAG GTG ACC GAC GGT ATC GG 3′, SEQ ID NO:193), and 100 ng/μl of 3′ ds-primer (5′CAG CGG ATA ACA ATT TCA CAC AGG GAG CTC CAC CGC GGT GGC GGC C3′, SEQ ID NO:194). After addition of the PCR product template, PCR was performed using the following program: 94° C., 4 minutes and 25 cycles of 94° C., 20 seconds; 65° C., 20 seconds; 72° C., 20 seconds; and 72° C. 4 minutes. The resulting amplified PCR product was gel purified.

The purified PCR product was sequenced using a standard protocol for ABI 3700 sequencing. Briefly, triplicate reactions in forward and reverse orientation (6 total reactions) were prepared, each reaction containing 5 μl of gel purified PCR product as template. In addition, the sequencing reactions contained 2 μl 2.5×sequencing buffer, 2 μl Big Dye Terminator mix, 1 μl of either the 5′ sequencing primer (5′CCC AGT CAC GAC GTT GTA AAA CG 3′, SEQ ID NO:195), or the 3′ sequencing primer (5′ TTT TTT TTT TTT TTT TTT V 3′, where V=A, C, or G, SEQ ID NO: 196) in a total volume of 10 μl.

In an alternate embodiment, the 3′ sequencing primer was the sequence 5′ GGT GGC GGC CGC AGG AAT TTT TTT TTT TTT TTT TT 3′, (SEQ ID NO: 197). PCR was performed using the following thermal cycling program: 96° C., 2 minutes and 29 cycles of 96° C., 15 seconds; 50° C., 15 seconds; 60° C., 4 minutes.

The sequence for REC1_{—}53 (SEQ ID NO:46) was determined by this method. Table 2 indicates that database searches indicated that this is a novel sequence.

In order to verify that the sequences determined by direct sequencing derived from the PCR product of interest, PCR primers were designed based on the sequences determined by direct sequencing, and PCR reactions were performed using the Ni TOGA PCR reaction products as substrate, as described above for the sequences cloned into the TOPO vector. In short, oligonucleotides were synthesized with the sequence G-A-T-C-G-A-A-T-C extended at the 3′ end with a partial MsI site (C-G-G) and an additional 18 nucleotides adjacent to the partial MspI site from the sequence determined by direct sequencing. The 5′ PCR primer (SEQ ID NO: 145) was paired with the fluorescent labeled universal 3′ PCR primer (SEQ ID NO:59) in PCR reactions with the NI TOGA PCR reaction product as template(see Table 3).

Some products, which were also differentially represented, appeared to migrate in positions that suggested that the products are novel based on sequence comparison data extracted from GenBank. In these cases, the PCR product was isolated, cloned into a TOPO vector (Invitrogen) and sequenced on both strands. In order to verify that the cloned product corresponds to the TOGA peak of interest, PCR primers were designed based on the determined sequence and PCR was performed using the cDNA produced in the first PCR reaction as substrate. Oligonucleotides were synthesized with the sequence G-A-T-C-G-A-A-T-C extended at the 3′ end with a partial MspI site (C-G-G), and an additional 18 adjacent nucleotides from the cloned PCR product or DST. For example, for the 288 b.p. product disclosed above, the 5′ PCR primer was G-A-T-C-G-A-A-T-C-C-G-G-A-A-G-C—C-G-C-G-C-A-T-C-A-C-T-G-A-G (SEQ ID NO: 86). This 5′ PCR primer was paired with the fluorescent labeled 3′ PCR primer (SEQ ID NO: 59) in PCRs using the cDNA produced in the first PCR reaction as substrate. The primers for these studies are shown in Table 3, below. In addition, Table 4 shows differentially expressed products having the partial sequence expected from the indicated corresponding sequence from GenBank as determined by the production of a PCR product using an extended 5′ PCR primer.

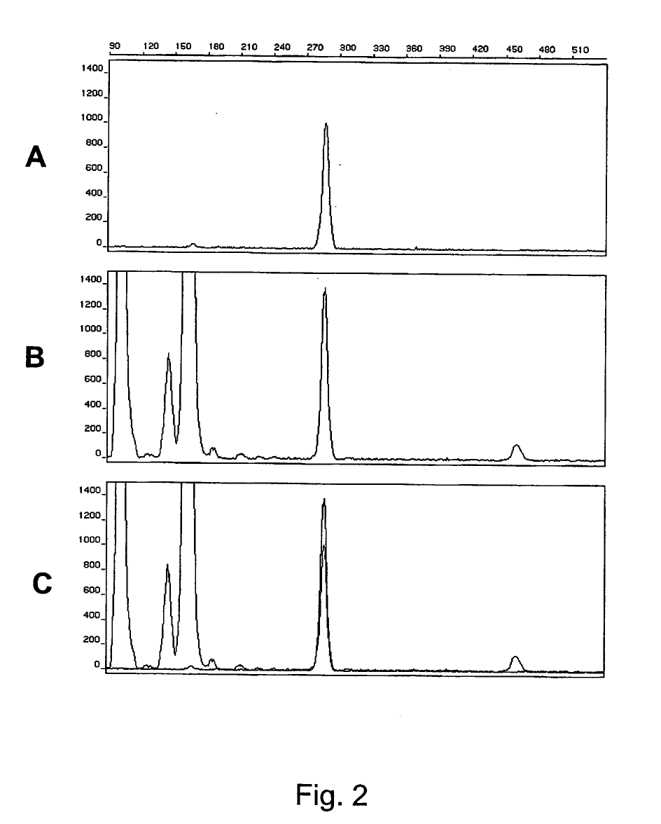

The products were separated by electrophoresis and the length of the clone was compared to the length of the original PCR product as shown in FIG. 2. The middle panel (B) shows the PCR products produced using the original PCR primers, SEQ ID NO: 61 and SEQ ID NO: 59 (compare to the panel in FIG. 1F). In FIG. 2A, the upper panel shows the length (as peak position) of the PCR product derived from the isolated clone as described above using the PCR primers SEQ ID NO: 86 and SEQ ID NO: 59. In the bottom panel, FIG. 2C, the traces from the top and middle panels are overlaid, demonstrating that the PCR product of the isolated and sequenced novel clone is the same length as the original PCR product.

As indicated by Table 1, the expression of REC1_{—}1 is up-regulated in a sustained manner in the aorta of rabbits with fatty atherosclerotic lesion development caused by hypercholesterolemia. As shown in Tables 2 and 3, REC1_{—}1 corresponds to a gene that encodes a tyrosine kinase binding protein. The REC1_{—}1 molecule may be useful as a diagnostic marker to indicate a predisposition for altered lipid or cholesterol transport (hypercholesterolemia) which could lead to the development of atherosclerosis. The results of these analyses also indicate that REC1_{—}1 may be a target substrate for the development of therapeutic agents.

In addition, several other molecules are up-regulated by the development of fatty atherosclerotic lesions. As shown in Table 1, clones REC1_{—}2, REC1_{—}8 and REC1_{—}1 are also up-regulated in the aorta of hypercholesterolemic rabbits in a sustained manner over the course of 8 weeks. For example, Table 1 shows AATC 108 (REC1^{—}2) is expressed at low levels on day 0 (control=177), which expression increases at day 14 (control=791) and remains elevated at 8 weeks (control=917). Similarly, CCCG 243 (REC1_{—}8) is expressed at low levels on day 0 (control=87) and shows increased expression at day 14 (control=566) and at week 8 (control=1370). GCAT 82 (REC1_{—}11) shows a similar pattern of expression in which expression is low at day 0 (control=319) and is increased at day 14 (control=135I) and at week 8 (control=3663).

Table 1 also shows that the expression of REC1_{—}3 and REC1_{—}10 is up-regulated early (at 14 days), but that the increased expression is not sustained. For example, ACGG 162 (REC1₁₃ 3) is expressed at a low level on day 0 (control=80), which expression increases at day 14 (control=688) and returns to a minimal level by week 8 (control=53). CTGA 390 (REC1_{—}10) shows a similar pattern of early up-regulated expression.

In addition, Table 1 shows that the expression of several clones, REC1_{—}6, REC1_{—}13, and REC1_{—}18, are up-regulated after a longer period of hypercholesterolemia. For example, ATCC 176 (REC1_{—}6) is up-regulated at 8 weeks, but not at two weeks. As shown in Table 1, REC1_{—}6 has a lower level of expression at day 0 (control=148) and day 14 (control=303) than at 8 weeks (control=1020). Similarly, GCCC 216 (REC1_{—}13) has a lower level of expression at day 0 (control=705) and day 14 (control=509) than at 0.8 weeks (control=4181). Likewise, TGGT 241 (REC1_{—}18) is expressed at a lower level on day 0 (control=188) and at day 14 (control=224) than at 8 weeks (control=799).

The above DST clones and at least part of the longer molecule corresponding to the DST clones can be useful in diagnosing or montoring the presence of, or the development of, atherosclerosis in hypercholesterolemia In one embodiment, the method of diagnosing or montoring the presence or development of atherosclerosis in hypercholesterolemia in a subject comprising detecting an alteration in expression of at least one polypeptide encoded by at least one polynucleotide selected from the group consisting of SEQ ID NO:1, SEQ ID NO:2, SEQ ID NO:3, SEQ ID NO:5, SEQ ID NO:7, SEQ ID NO:8, SEQ ID NO:9, SEQ ID NO:11 and SEQ ID NO:21.

### EXAMPLE 2

### RT-PCR Analysis of Polynucleotides Up-Regulated During Hypercholesterolemia

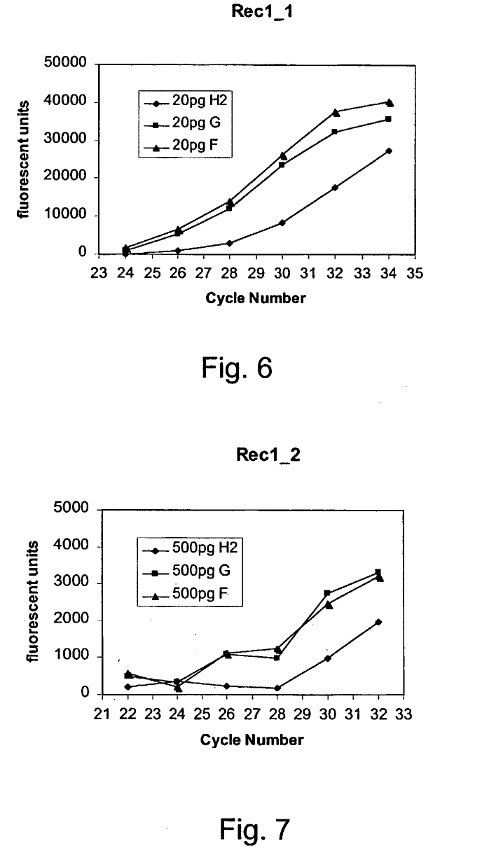

Nineteen of the isolated DST clones were further validated using RT-PCR analyses; the data are presented in FIGS. 6-24 and in Tables 5-23, below. The DST clones are summarized in Table 24. The primers used for RT-PCR are listed in Table 25. For each DST examined, the optimal annealing temperature and reagent conditions were determined for the corresponding set of primers (see Table 25) based on results from a preliminary experiment. In eight separate reactions, each set of primers was assayed to find the optimal conditions by adjusting the following four parameters: primer concentration, dNTP concentration, MgCl₂ concentration, and number of Taq polymerase units. Once optimal conditions were determined, each DST was run in duplicate multiple simultaneous reactions which usually included at least four dilutions of template (cDNA library), plus control reactions lacking template, and six sequential data points for numbers of cycles.

Reactions were performed using “Hot Start” PCR with the Clontech TaqStart antibody system (Cat. #5400-1). Each reaction contained 1 ml of the cDNA library dilution as template, determined amounts of AmpliTaq DNA polymerase (cat. #N808-0156), MgCl₂, dNTPs (GibcoBRL cat. #10297-018), primer, and Clontech TaqStart Antibody in a 20 ml final reaction volume using 10×Taq buffer II (without MgCl₂). Typically, a master mix containing all components except the template was prepared and aliquoted. Various templates were then added to these master mix samples and 20 ml volumes were subsequently dispensed into individual reaction tubes. At various times during the PCR run, tubes were removed sequentially on a predetermined schedule in order to quantitate expression of the target DST over a “window” of cycle times. After amplification, the samples were quantified via fluorimetry.

PCR was performed at annealing temperatures that were five degrees above the average melting temperature of each primer pair. For primers greater than 10 bases (in a 0.05M salt solution), melting temperature was calculated using the following formula: Tm=59.9+41[% GC]−[675/Primer Length], where %GC is the decimal value. Following determination of the average melting temperature of the primer pair, the annealing temperature was determined by adding five degrees to the average melting temperature. PCR was performed using the following program: 1) 95 degrees Celsius, 3 minutes; 2) 95 degrees Celsius, 30 seconds; 3) TM+5 degrees Celsius, 30 seconds; 4) 72 degrees Celsius, for a time dependent on target length at 16 bp/second; 5) repeat steps 2-4 33 more cycles; 6) 72 degrees Celsius, 3 minutes; 7) 14 degrees Celsius, forever.

Following temperature cycling, 2 ml of the PCR reaction was added to 140 ml of a 1:280 dilution of PicoGreen (Molecular Probes cat. #P-11495 (10×100 ml)) in TE pH 7.5 in a 96-well Costar UV microtiter plate (Fisher cat. #07-200623). The samples were mixed gently for 1.5 minutes and allowed to equilibrate at room temperature in the dark for 15 minutes. The concentration of the PCR products was quantified by fluorimetry using a PerSeptive Biosystems CytoFluor series 4000 multi-well plate reader.

Background fluorescence was determined by using duplicate control samples that were cycled with all reaction components except the template. The mean value from these duplicate background control samples was subtracted from the corresponding experimental values prior to analyzing results. The sensitivity of the PicoGreen dsDNA assay is reported to be 250 pg/ml (50 pg dsDNA in a 200 ml assay volume) using a fluorescence microplate reader such as was used in these measurements.

DST clone Rec1_{—}1 was one of the clones further evaluated by RT-PCR. The TOGA analysis of clone Rec1_{—}1, identified as MspI AAGC 288, is discussed above and illustrated in FIGS. 1 and 2. The results of the quantified RT-PCR for Rec1_{—}1 are shown in FIG. 6 and in Table 5 (normalized to the normocholesterolemic control value (“H2”) at each time point). FIG. 6 is a graphical representation of the results of RT-PCR using 20 pg of clone REC1_{—}1 template, in which the amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

TABLE 5
Relative Abundance Rec1_1
20 pg template

| Cycle | 20 pg H2 | 20 pg G | 20 pg F |
|---|---|---|---|
| 24 | 1.0 | 920.5 | 1622 |
| 26 | 1.0 | 6.3 | 7.7 |
| 28 | 1.0 | 4.3 | 4.9 |
| 30 | 1.0 | 2.8 | 3.2 |
| 32 | 1.0 | 1.8 | 2.1 |
| 34 | 1.0 | 1.3 | 1.5 |
| TOGA | 1.0 | 6.7 | 6.9 |

As noted above, TOGA analysis showed that the expression of REC1_{—}1 is up-regulated in a sustained manner in the aorta of rabbits with fatty atherosclerotic lesion development caused by hypercholesterolemia. The expression pattern was verified in the RT-PCR study, which showed that the expression of Rec1_{—}1 at 8 weeks is elevated both in hypercholesterolemic animals and in hypercholesterolemic animals receiving lercanidipine racemate

TOGA analysis of DST clone Rec1_{—}2, identified as MspI AATC 108 showed expression at low levels on day 0 followed by increased expression at day 14 and at 8 weeks (Table 1). The results of the quantified RT-PCR for Rec1_{—}2 are shown in FIG. 7 and in Table 6. FIG. 7 is a graphical representation of the results of RT-PCR using 500 pg of clone REC1_{—}2 template, in which the amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

TABLE 6
Relative Abundance Rec1_2
500 pg template

| Cycle | 500 pg H2 | 500 pg G | 500 pg F |
|---|---|---|---|
| 22 | 1.0 | 2.4 | 2.7 |
| 24 | 1.0 | 0.9 | 0.6 |
| 26 | 1.0 | 4.9 | 5.0 |
| 28 | 1.0 | 5.2 | 6.6 |
| 30 | 1.0 | 2.8 | 2.5 |
| 32 | 1.0 | 1.7 | 1.6 |
| TOGA | 1.0 | 5.2 | 6.5 |

As noted above, TOGA analysis showed that the expression of REC1_{—}2 is up-regulated in a sustained manner in the aorta of rabbits with fatty atherosclerotic lesion development caused by hypercholesterolemia. The expression pattern was verified in the RT-PCR study, which showed that the expression of Rec1_{—}2 at 8 weeks is elevated both in hypercholesterolemic animals and in hypercholesterolemic animals receiving lercanidipine racemate

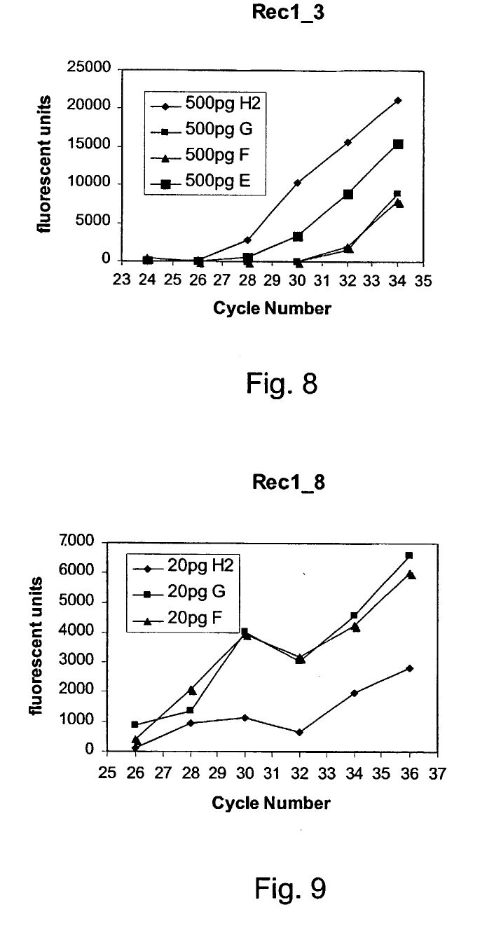

TOGA analysis of DST clone Rec1_{—}3, identified as MspI ACGG 162, showed expression at low levels on day 0 followed by increased expression at day 14 and at 8 weeks (Table 1). The results of the quantified RT-PCR for Rec1_{—}3 are shown in FIG. 8 and in Table 7. FIG. 8 is a graphical representation of the results of RT-PCR using 500 pg of clone REC1_{—}3 template, in which the amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic (large filled squares, “E”), hypercholesterolemic, eight weeks (filled squares, “G”) and hypercholesterolemic plus lercanidipine racemate, eight weeks (filled triangles, “F”).

TABLE 7
Relative Abundance Rec1_3
500 pg template

| Cycle | 500 pg H2 | 500 pg G | 500 pg F | 500 pg E |
|---|---|---|---|---|
| 26 | 1.0 | 0.0 | 0.0 | 0.0 |
| 28 | 1.0 | 0.0 | 0.0 | 0.2 |
| 30 | 1.0 | 0.0 | 0.0 | 0.3 |
| 32 | 1.0 | 0.1 | 0.1 | 0.6 |
| 34 | 1.0 | 0.4 | 0.4 | 0.7 |
| TOGA | 1.0 | 0.7 | 0.8 | 8.6 |

TOGA analysis showed that the expression of REC1_{—}3 is up-regulated in a transient manner in the aorta of rabbits with fatty atherosclerotic lesion development caused by hypercholesterolemia. The expression pattern was verified in the RT-PCR study, which showed that the expression of Rec1_{—}3 at 8 weeks is lower both in hypercholesterolemic animals and hypercholesterolemic animals receiving lercanidipine racemate than in hypercholesterolemic animals at 14 days.

TOGA analysis of DST clone Rec1_{—}8, identified as MspI CCCG 243, showed expression at low levels on day 0 followed by increased expression at day 14 and at 8 weeks (Table 1). The results of the quantified RT-PCR for Rec1_{—}3 are shown in FIG. 9 and in Table 8. FIG. 9 is a graphical representation of the results of RT-PCR using 20 pg of clone REC1_{—}8 template, in which the amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

TABLE 8
Relative Abundance Rec1_8
20 pg template

| Cycle | 20 pg H2 | 20 pg G | 20 pg F |
|---|---|---|---|
| 26 | 1.0 | 9.5 | 4.6 |
| 28 | 1.0 | 1.5 | 2.2 |
| 30 | 1.0 | 3.6 | 3.5 |
| 32 | 1.0 | 4.5 | 4.8 |
| 34 | 1.0 | 2.3 | 2.1 |
| 36 | 1.0 | 2.3 | 2.1 |
| TOGA | 1.0 | 15.7 | 13.1 |

TOGA analysis showed that the expression of REC1_{—}8 is up-regulated in a sustained manner in the aorta of rabbits with fatty atherosclerotic lesion development caused by hypercholesterolemia. The expression pattern was verified in the RT-PCR study, which showed that the expression of Rec1_{—}8 at 8 weeks is elevated both in hypercholesterolemic animals and in hypercholesterolemic animals receiving lercanidipine racemate.

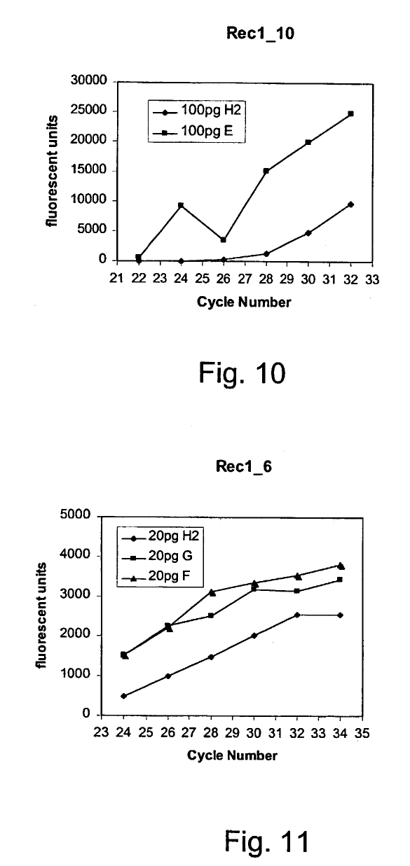

TOGA analysis of DST clone Rec1_{—}10, identified as MspI CTGA 390, showed expression at low levels on day 0 followed by increased expression at day 14 that was less elevated at 8 weeks (Table 1). The results of the quantified RT-PCR for Rec1_{—}10 are shown in FIG. 10 and in Table 9. FIG. 10 is a graphical representation of the results of RT-PCR using 100 pg of clone REC1_{—}10 template, in which the amount of PCR product (measured in arbitrary fluorescence units) is plotted against number of cycles, where polyA enriched mRNA was extracted from aortas of rabbits that were normocholesterolemic (filled diamonds, “H2”) and hypercholesterolemic (filled squares, “E”).

TABLE 9
Relative Abundance Rec1_10
100 pg template

| Cycle | 100 pg H2 | 100 pg E |
|---|---|---|
| 22 | 1.0 | 558.0 |
| 24 | 1.0 | 9300.0 |
| 26 | 1.0 | 12.4 |
| 28 | 1.0 | 11.3 |
| 30 | 1.0 | 4.2 |
| 32 | 1.0 | 2.6 |
| TOGA | 1.0 | 14.9 |

TOGA analysis showed that the expression of REC1_{—}10 is up-regulated several-fold at 14 days in the aorta of rabbits with fatty atherosclerotic lesion development caused by hypercholesterolemia. A consistent expression pattern was found in the RT-PCR study, which showed that the expression of Rec1_{—}10 was elevated at 14 days.

TOGA analysis of DST clone Rec1_{—}6, identified as MspI ATCC 176, showed expression at low levels on day 0 followed by increased expression at day 14 and further elevated expression at 8 weeks (Table 1). The results of the quantified RT-PCR for Rec1_{—}6 are shown in FIG. 11 and in Table 10. FIG. 11 is a graphical representation of the results of RT-PCR using 20 pg of clone REC1_{—}6 template, in which the amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

TABLE 10
Relative Abundance Rec1_6
20 pg template

| Cycle | 20 pg H2 | 20 pg G | 20 pg F |
|---|---|---|---|
| 24 | 1.0 | 3.1 | 3.1 |
| 26 | 1.0 | 2.3 | 2.2 |
| 28 | 1.0 | 1.7 | 2.1 |
| 30 | 1.0 | 1.6 | 1.7 |
| 32 | 1.0 | 1.2 | 1.4 |
| 34 | 1.0 | 1.3 | 1.5 |
| TOGA | 1.0 | 6.9 | 8.8 |

TOGA analysis showed that the expression of Rec1_{—}6 is up-regulated in a sustained manner in the aorta of rabbits with fatty atherosclerotic lesion development caused by hypercholesterolemia. The expression pattern was verified in the RT-PCR study, which showed that the expression of Rec1_{—}6 at 8 weeks is elevated in hypercholesterolemic animals and somewhat more elevated in hypercholesterolemic animals receiving lercanidipine racemate.

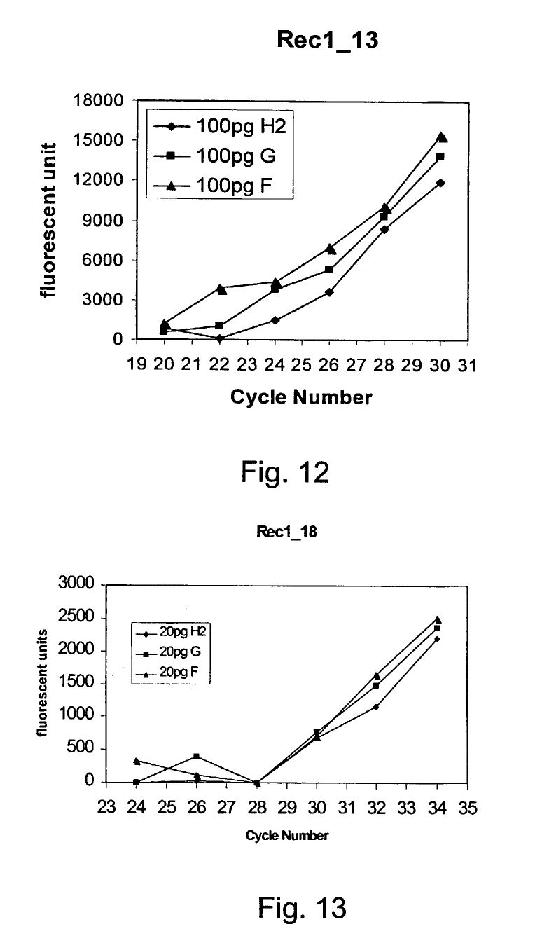

TOGA analysis of DST clone Rec1_{—}13, identified as MspI GCCC 216, showed expression at moderate levels on day 0 followed by somewhat decreased expression at day 14 and substantially elevated expression at 8 weeks (Table 1). The results of the quantified RT-PCR for Rec1_{—}13 are shown in FIG. 12 and in Table 11. FIG. 12 is a graphical representation of the results of RT-PCR using 100 pg of clone REC1_{—}13 template, in which the amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

TABLE 11
Relative Abundance Rec1_13
100 pg template

| Cycle | 100 pg H2 | 100 pg G | 100 pg F |
|---|---|---|---|
| 20 | 1.0 | 0.7 | 1.4 |
| 22 | 1.0 | 9.3 | 33.7 |
| 24 | 1.0 | 2.6 | 3.0 |
| 26 | 1.0 | 1.5 | 1.9 |
| 28 | 1.0 | 1.1 | 1.2 |
| 30 | 1.0 | 1.2 | 1.3 |
| TOGA | 1.0 | 5.9 | 5.9 |

TOGA analysis showed that the expression of Rec1_{—}13 is up-regulated at 8 weeks in the aorta of rabbits with the fatty atherosclerotic lesion development caused by hypercholesterolemia. The expression pattern was verified in the RT-PCR study, which shown that the expression of Rec1_{—}13 at 8 weeks is elevated both in hypercholesterolemic animals and in hypercholesterolemic animals receiving lercanidipine racemate.

TOGA analysis of DST clone Rec1_{—}18, identified as MspI TGGT 241, showed expression at moderate levels on day 0 followed by somewhat increased expression at day 14 and substantially elevated expression at 8 weeks (Table 1). The results of the quantified RT-PCR for Rec1_{—}18 are shown in FIG. 13 and in Table 12. FIG. 13 is a graphical representation of the results of RT-PCR using 20 pg of clone REC1_{—}18 template, in which the amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

TABLE 12
Relative Abundance Rec1_18
20 pg template

| Cycle | 20 pg H2 | 20 pg G | 20 pg F |
|---|---|---|---|
| 24 | 1.0 | 0.5 | 169.0 |
| 26 | 1.0 | 18.8 | 5.8 |
| 28 | 1.0 | 1.0 | 1.0 |
| 30 | 1.0 | 1.1 | 1.0 |
| 32 | 1.0 | 1.3 | 1.4 |
| 34 | 1.0 | 1.1 | 1.1 |
| TOGA | 1.0 | 4.3 | 5.0 |

TOGA analysis showed that the expression of Rec1_{—}18 is up-regulated at 8 weeks in the aorta of rabbits with fatty atherosclerotic lesion development caused by hypercholesterolemia. The RT-PCR study showed that the expression of Rec1_{—}18 at 8 weeks is somewhat elevated both in hypercholesterolemic animals and in hypercholesterolemic animals receiving lercanidipine racemate.

### EXAMPLE 3

### Identification and Characterization of Polynucleotides Down-Regulated by Fatty Lesion Development

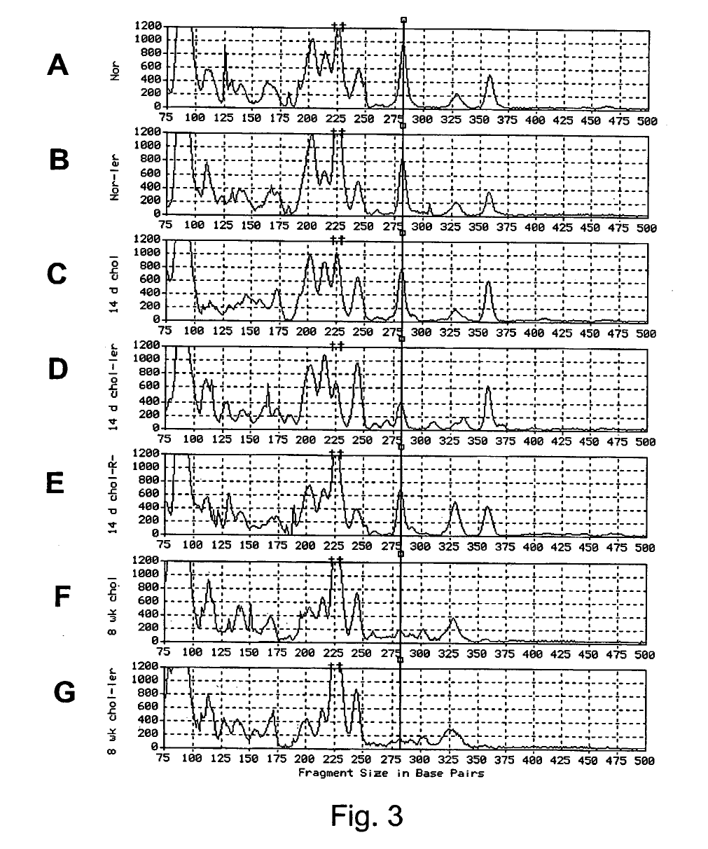

An example of a polynucleotide that is down-regulated by fatty atherosclerotic lesion development is shown in FIGS. 3-5. In FIG. 3, a peak at about 282 is indicated, identified by digital address MspI CACA 282 when a 5′ PCR primer (SEQ ID NO: 66) was paired with SEQ ID NO: 59 to produce the panel of PCR products. The PCR product was cloned and sequenced as described in Example 1. To verify that the isolated clone (SEQ ID NO: 6) corresponds to the TOGA peak of interest, oligonucleotides were synthesized with the sequence G-A-T-C-G-A-A-T-C extended at the 3′ end with a partial MspI site (C-G-G), and an additional 18 adjacent nucleotides from the cloned PCR product or DST. In this case, the 5′ PCR primer was G-A-T-C-G-A-A-T-C-C-G-G-C-A-C-A-C-G-G-G-C-G-C-A-A-G-A-A-G-A (SEQ ID NO: 91). This 5′ PCR primer was paired with the fluorescently labeled 3′ PCR primer (SEQ ID NO: 59) in PCRs using the cDNA produced in the first PCR reaction as substrate.

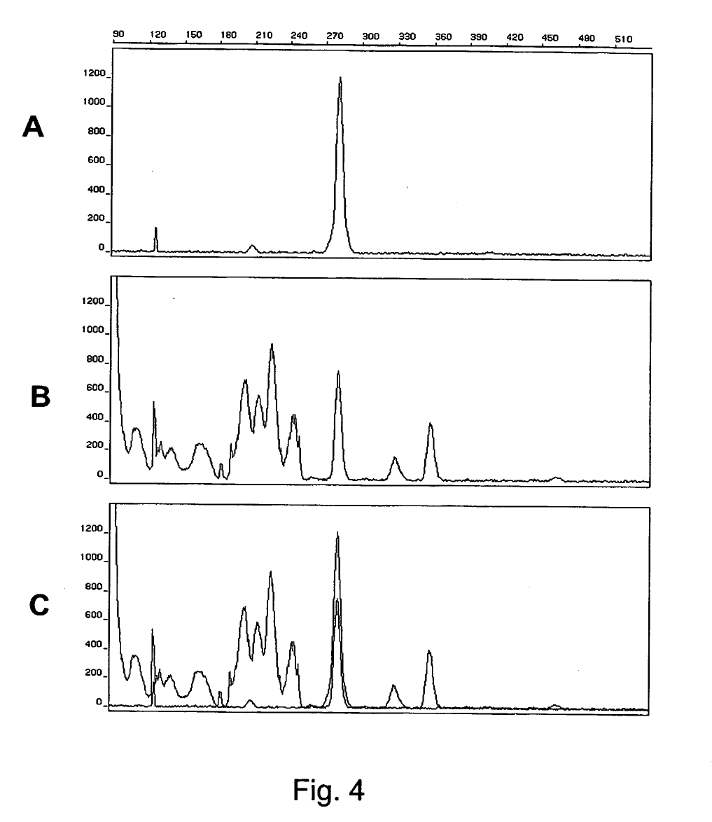

In FIG. 4, the upper panel (4A) shows the PCR product produced using the original PCR primers, SEQ ID NO: 66 and SEQ ID NO: 59. In FIG. 4B, the middle panel shows the length (as peak position) of the PCR product derived from the isolated clone as described in Example 1 (using SEQ ID NO: 91 and SEQ ID NO: 59). In the bottom panel FIG. 4C, the traces from the top and middle panels are overlaid, demonstrating that the PCR product of the isolated and sequenced novel clone is the same length as the original PCR product.

As shown in Table 1, the DNA molecule identified by the digital address MspI CACA 282 (clone REC1_{—}7), is further characterized as having a 5′ terminus partial nucleotide sequence of CGGCACA and a digital address length of 282 b.p. REC1_{—}7 is further described as being down-regulated during fatty lesion development. REC1_{—}7 is expressed at higher levels in control aorta at day 0 (976, FIG. 3A) and lercanidipine-treated aorta at day 0 (863, FIG. 3B) than in control aorta at day 14 of cholesterol administration (787, FIG. 3C), lercanidipine-treated aorta at day 14 of cholesterol administration (417, FIG. 3D), and (R)-lercanidipine-treated aorta at day 14 (683, FIG. 3E), as well as control aorta at week 8 of cholesterol administration (199, FIG. 3F) and lercanidipine-treated aorta at week 8 of cholesterol administration (159, FIG. 3G). Thus, the vertical index line indicates a PCR product of about 282 b.p. that shows greater expression in the aorta of rabbits fed a normal diet than those fed a high cholesterol diet.

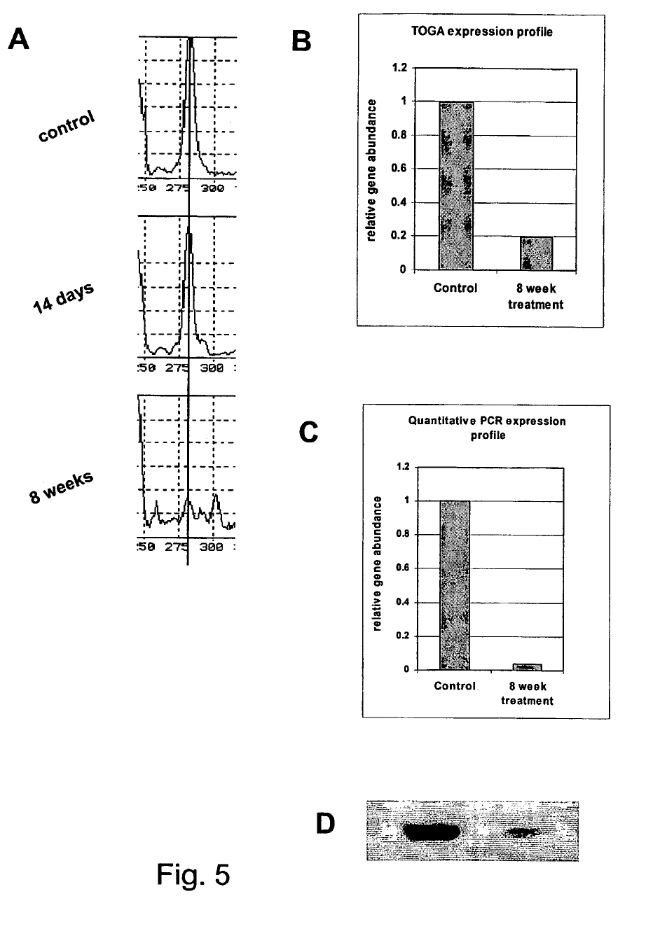

The differential gene expression of REC1_{—}7 is confirmed by the data shown in FIGS. 5A-D. FIG. 5A shows the TOGA analysis presented in FIG. 3 for control aorta (no lercanidipine treatment) at day 0, day 14, and week 8 of cholesterol administration. FIG. 5B shows the relative abundance of the REC1_{—}7 product found in control aorta at day 0 and week 8 as determined from the TOGA graphical user interface (GUI) intensities. FIG. 5C shows the relative abundance of the REC1_{—}7 product found in control aorta at day 0 and week 8, as determined by quantitative PCR performed from sample cDNA using internal primers of known REC1_{—}7 sequence (SEQ ID NO:124 and 125) to generate a gene-specific PCR fragment. The gel image below the graph visually depicts the REC1_{1—}products formed in the quantitative PCR reaction.

The full-length gene comprising REC1_{—}7 is presently unidentified. Interestingly, REC1_{—}7 is down-regulated in the aorta during fatty lesion development.

In addition, several other molecules are also down-regulated by the development of fatty lesions. As shown in Table 1, REC1_{—}5, REC1_{—}16, REC1_{—}17, REC1_{—}19, REC1_{—}20, and REC1_{—}21 are down-regulated in the aorta of hypercholesterolemic rabbits in a sustained manner over the course of 8 weeks. For example, Table 1 shows that AGTG 184 (REC1_{—}5) is expressed at higher levels on day 0 (control=1086) than on day 14 (control=433) and that its expression continues to decrease at week 8 (control=285). Similarly, the expression of TCGG 195 (REC1_{—}16) decreases over time under conditions of fatty lesion development induced by hypercholesterolemia. The expression is higher at day 0 (control=2497) than at day 14 (control=1247) or week 8 (control=479). Likewise, the expression of TCGT 199 (REC1_{—}17) is higher at day 0 (control=1782) than at day 14 (control=832) or week 8 (control=272). For TGTT 393 (REC1_{—}19), the expression is higher at day 0 (control=687) than at day 14 (control=322) or week 8 (control=96). Also, for TTAC 210 (REC1_{—}20) and TTCC 165 (REC1_{—}121), the expression is higher at day 0 (control 874; 3144) than at day 14 (control=393; 1203) or week 8 (control=210; 636).

Table 1 also shows that the expression of REC1_{—}15 is down-regulated early after fatty lesion development (at 14 days), but that the decreased expression is not sustained. For example, the level of expression of TCAG 76 (REC1_{—}15) is greatly decreased at day 14 of hypercholesterolemia (control, day 0=904; control, day 14=77) and then increased at 8 weeks of cholesterol administration (control=356).

The above DST clones and at least part of the longer molecule corresponding to the DST clones can be useful in diagnosing or montoring the presence of, or the development of, atherosclerosis in hypercholesterolemia In one embodiment, the method of diagnosing or montoring the presence or development of atherosclerosis in hypercholesterolemia in a subject comprising detecting an alteration in expression of at least one polypeptide encoded by at least one polynucleotide selected from the group consisting of SEQ ID NO:4, SEQ ID NO:6, SEQ ID NO:13, SEQ ID NO:14, SEQ ID NO:16, SEQ ID NO:17 and SEQ ID NO:18.

In another embodiment, resolution or accuracy can be impoved by comparing the alteration in expression of more than one gene. In one embodiment, the present invention provides a method of diagnosing or montoring the presence of development of atherosclerosis in hypercholesterolemia in a subject comprising comparing an alteration in expression of at least one polypeptide encoded by at least one polynucleotide selected from the group consisting of SEQ ID NO:1, SEQ ID NO:2, SEQ ID NO:3, SEQ ID NO:5, SEQ ID NO:7, SEQ ID NO:8, SEQ ID NO:9, SEQ ID NO:11 and SEQ ID NO:21 to an alteration in expression of at least one polypeptide encoded by at least one polynucleotide selected from the group consisting of SEQ ID NO:4, SEQ ID NO:6, SEQ ID NO:13, SEQ ID NO:14, SEQ ID NO:16, SEQ ID NO:17 and SEQ ID NO:18.

### EXAMPLE 4

### RT-PCR Analysis of Polynucleotides Down-Regulated During Hypercholesterolemia

Several DST clones that showed down-regulation in the TOGA analysis described in Example 3, above, were studied further using the RT-PCR techniques and analysis of Example 2.

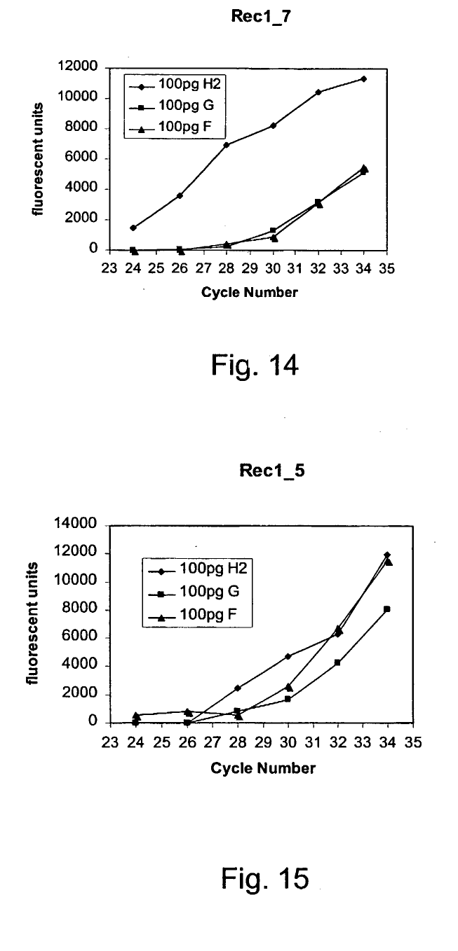

TOGA analysis of DST clone Rec1_{—}7, identified as CACA 282, was described in detail in Example 3 and FIGS. 3-5, showed expression at substantial levels on day 0 followed by decreased expression at day 14 and further decreased expression at 8 weeks (Table 1). The results of the quantified RT-PCR for Rec1_{—}7 are shown in FIG. 14 and in Table 13. FIG. 14 is a graphical representation of the results of RT-PCR using 100 pg of clone REC1_{—}7 template, in which the amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

TABLE 13
Relative Abundance Rec1_7
100 pg template

| Cycle | 100 pg H2 | 100 pg G | 100 pg F |
|---|---|---|---|
| 24 | 1.0 | 0.00 | 0.00 |
| 26 | 1.0 | 0.02 | 0.00 |
| 28 | 1.0 | 0.04 | 0.06 |
| 30 | 1.0 | 0.15 | 0.10 |
| 32 | 1.0 | 0.3 | 0.3 |
| 34 | 1.0 | 0.5 | 0.5 |
| TOGA | 1.0 | 0.2 | 0.2 |

TOGA analysis showed that the expression of Rec1_{—}7 is down-regulated at 8 weeks in the aorta of rabbits with fatty atherosclerotic lesion development caused by hyper-cholesterolemia. The expression pattern was verified in the RT-PCR study, which showed that the expression of Rec1_{—}7 at 8 weeks is down-regulated both in hypercholesterolemic animals and in hypercholesterolemic animals receiving lercanidipine racemate.

TOGA analysis of DST clone Rec1_{—}5, identified as AGTG 184, showed expression at high levels on day 0 followed by decreased expression at day 14 and further decreased expression at 8 weeks (Table 1). The results of the quantified RT-PCR for Rec1_{—}5 are shown in FIG. 15 and in Table 14. FIG. 15 is a graphical representation of the results of RT-PCR using 100 pg of clone REC1_{—}5 template, in which the amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

TABLE 14
Relative Abundance Rec1_5
100 pg template

| Cycle | 100 pg H2 | 100 pg G | 100 pg F |
|---|---|---|---|
| 24 | 1.0 | 1.0 | 512.5 |
| 26 | 1.0 | 1.0 | 805.0 |
| 28 | 1.0 | 0.3 | 0.2 |
| 30 | 1.0 | 0.3 | 0.6 |
| 32 | 1.0 | 0.7 | 1.1 |
| 34 | 1.0 | 0.7 | 1.0 |
| TOGA | 1.0 | 0.3 | 0.2 |

TOGA analysis showed that the expression of Rec1_{—}5 is down-regulated at 8 weeks in the aorta of rabbits with fatty atherosclerotic lesion development caused by hyper-cholesterolemia. The expression pattern was verified in the RT-PCR study, which showed that the expression of Rec1_{—}5 at 8 weeks is decreased in hypercholesterolemic animals and in hypercholesterolemic animals receiving lercanidipine racemate at 28-30 cycles.

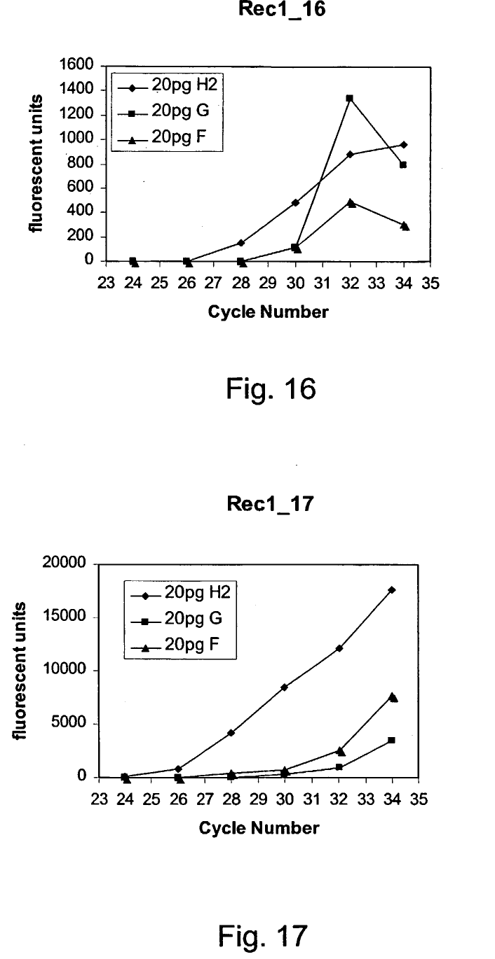

TOGA analysis of DST clone Rec1_{—}16, identified as TCGG 195, showed expression at high levels on day 0 followed by decreased expression at day 14 and further decreased expression at 8 weeks (Table 1). The results of the quantified RT-PCR for Rec1_{—}16 are shown in FIG. 16 and in Table 15. FIG. 16 is a graphical representation of the results of RT-PCR using 20 pg of clone REC1_{—}16 template, in which the amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

TABLE 15
Relative Abundance Rec1_16
20 pg template

| Cycle | 20 pg H2 | 20 pg G | 20 pg F |
|---|---|---|---|
| 28 | 1.0 | 0.0 | 0.0 |
| 30 | 1.0 | 0.2 | 0.2 |
| 32 | 1.0 | 1.5 | 0.6 |
| 34 | 1.0 | 0.8 | 0.3 |
| TOGA | 1.0 | 0.2 | 0.2 |

TOGA analysis showed that the expression of Rec1_{—}16 is down-regulated at 8 weeks in the aorta of rabbits with fatty atherosclerotic lesion development caused by hyper-on cholesterolemia. The expression pattern was verified in the RT-PCR study, which showed that the expression of Rec1_{—}16 at 8 weeks is decreased in hypercholesterolemic animals and in hypercholeserolemic animals receiving lercanidipine.

TOGA analysis of DST clone Rec1_{—}17, identified as TCGT 199, showed expression at high levels on day 0 followed by decreased expression at day 14 and further decreased expression at 8 weeks (Table 1). The results of the quantified RT-PCR for Rec1_{—}17 are shown in FIG. 17 and in Table 16. FIG. 17 is a graphical representation of the results of RT-PCR using 20 pg of clone REC1_{—}17 template, in which the amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

TABLE 16
Relative Abundance Rec1_17
20 pg template

| Cycle | 20 pg H2 | 20 pg G | 20 pg F |
|---|---|---|---|
| 24 | 1.0 | 0.16 | 0.01 |
| 26 | 1.0 | 0.00 | 0.00 |
| 28 | 1.0 | 0.00 | 0.09 |
| 30 | 1.0 | 0.03 | 0.08 |
| 32 | 1.0 | 0.08 | 0.21 |
| 34 | 1.0 | 0.20 | 0.43 |
| TOGA | 1.0 | 0.15 | 0.15 |

TOGA analysis showed that the expression of Rec1_{—}17 is down-regulated at 8 weeks in the aorta of rabbits with fatty atherosclerotic lesion development caused by hyper-cholesterolemia. The expression pattern was verified in the RT-PCR study, which showed that the expression of Rec1_{—}17 at 8 weeks is decreased both in hypercholesterolemic animals and in hypercholesterolemic animals receiving lercanidipine.

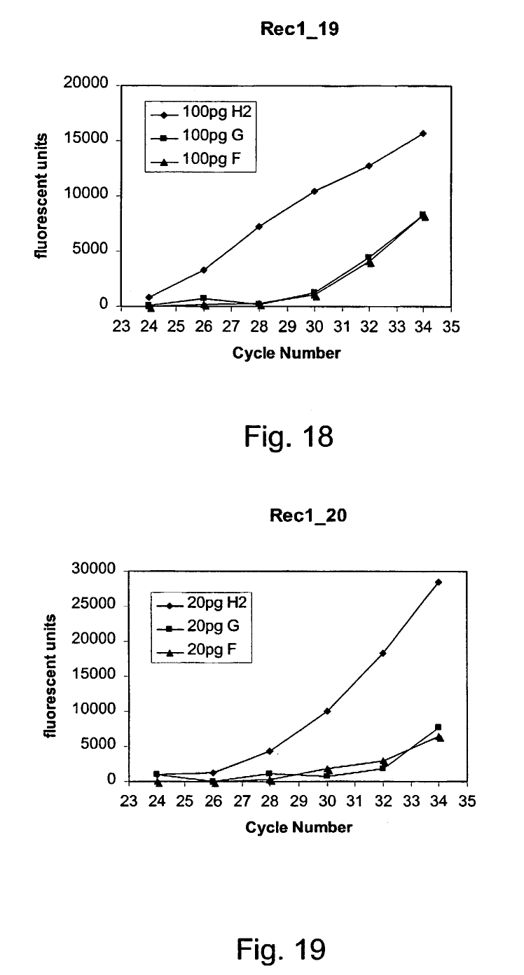

TOGA analysis of DST clone Rec1_{—}19 identified as TGTT 393, showed expression at moderate levels on day 0 followed by decreased expression at day 14 and further decreased expression at 8 weeks (Table 1). The results of the quantified RT-PCR for Rec1_{—}19 are shown in FIG. 18 and in Table 17. FIG. 18 is a graphical representation of the results of RT-PCR using 100 pg of clone REC1_{—}19 template, in which the amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lereanidipine racemate, eight weeks (filled triangles, “F”).

TABLE 17
Relative Abundance Rec1_19
1000 pg template

| Cycle | 100 pg H2 | 100 pg G | 100 pg F |
|---|---|---|---|
| 24 | 1.0 | 0.2 | 0.0 |
| 26 | 1.0 | 0.2 | 0.0 |
| 28 | 1.0 | 0.0 | 0.0 |
| 30 | 1.0 | 0.1 | 0.1 |
| 32 | 1.0 | 0.3 | 0.3 |
| 34 | 1.0 | 0.5 | 0.5 |
| TOGA | 1.0 | 0.1 | 0.2 |

TOGA analysis showed that the expression of Rec1_{—}19 is substantially down-regulated at 8 weeks in the aorta of rabbits with fatty atherosclerotic lesion development caused by hyper-cholesterolemia. The expression pattern was verified in the RT-PCR study, which showed that the expression of Rec1_{—}19 at 8 weeks is decreased both in hypercholesterolemic animals and in hypercholesterolemic animals receiving lercanidipine.

TOGA analysis of DST clone Rec1_20, identified as TTAC 210, showed expression at moderate levels on day 0 followed by decreased expression at day 14 and further decreased expression at 8 weeks (Table 1). The results of the quantified RT-PCR for Rec1_{—}20 are shown in FIG. 19 and in Table 18. FIG. 19 is a graphical representation of the results of RT-PCR using 20 pg of clone REC1_{—}20 template, in which the amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

TABLE 18
Relative Abundance Rec1_20
20 pg template

| Cycle | 20 pg H2 | 20 pg G | 20 pg F |
|---|---|---|---|
| 26 | 1.0 | 0.0 | 0.0 |
| 28 | 1.0 | 0.3 | 0.1 |
| 30 | 1.0 | 0.1 | 0.2 |
| 32 | 1.0 | 0.1 | 0.2 |
| 34 | 1.0 | 0.3 | 0.2 |
| TOGA | 1.0 | 0.2 | 0.3 |

TOGA analysis showed that the expression of Rec1_{—}20 is substantially down-regulated at 8 weeks in the aorta of rabbits with fatty atherosclerotic lesion development caused by hyper-cholesterolemia. The expression pattern was verified in the RT-PCR study, which showed that the expression of Rec1_{—}20 at 8 weeks is decreased both in hypercholesterolemic animals and in hypercholesterolemic animals receiving lercanidipine.

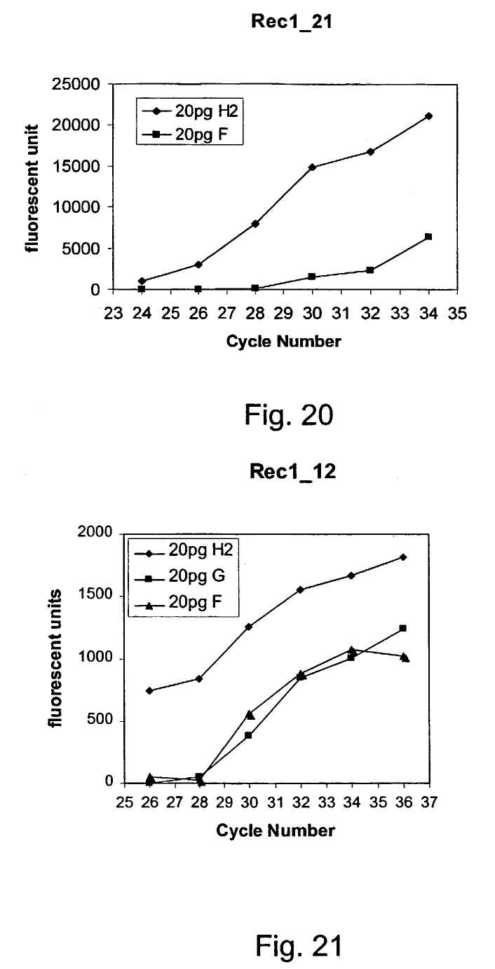

TOGA analysis of DST clone Rec_{—}21, identified as TTCC 165, showed expression at high levels on day 0 followed by decreased expression at day 14 and further decreased expression at 8 weeks (Table 1). The results of the quantified RT-PCR for Rec1_{—}21 are shown in FIG. 20 and in Table 19. FIG. 20 is a graphical representation of the results of RT-PCR using 20 pg of clone REC1_{—}21 template, in which the amount of PCR product (measured in arbitrary fluorescence units) is plotted against number of cycles, where polyA enriched mRNA was extracted from aortas or rabbits that were normocholesterolemic (filled diamonds, “H2”) and hypercholestrolemic plus lercanidipine racemate, eight weeks (filled squares, “F”).

TABLE 19
Relative Abundance Rec1_21
20 pg template

| Cycle | 20 pg H2 | 20 pg F |
|---|---|---|
| 24 | 1.0 | 0.0 |
| 26 | 1.0 | 0.0 |
| 28 | 1.0 | 0.0 |
| 30 | 1.0 | 0.1 |
| 32 | 1.0 | 0.1 |
| 34 | 1.0 | 0.3 |
| TOGA | 1.0 | 0.2 |

TOGA analysis showed that the expression of Rec1_{—}21 is down-regulated by about ten-rabbits fold at 8 weeks in the aorta of rabbits with fatty atherosclerotic lesion development caused by hyper-cholestrolemic. The expression pattern was verified in the RT-PCR study, which showed that the expression of Rec1_{—}21 at 8 weeks is substantially decreased in hypercholestrolemic animals receiving lercanidipine.

TOGA analysis of DST clone Rec1_{—}12, identified as GCCC 232, showed expression at high levels on day 0 followed by somewhat decreased expression at day 14 and further substantially dereased expression at 8 weeks (Table 1). The results of the quantified RT-PCR for Rec1_{—}12 are shown in FIG. 21 and in Table 20. FIG. 21 is a graphical representation of the results of RT-PCR using 20 pg of clone REC1_{—}12 template, in which the amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

TABLE 20
Relative Abundance Rec1_12
20 pg template

| Cycle | 20 pg H2 | 20 pg G | 20 pg F |
|---|---|---|---|
| 26 | 1.0 | 0.0 | 0.1 |
| 28 | 1.0 | 0.1 | 0.0 |
| 30 | 1.0 | 0.3 | 0.4 |
| 32 | 1.0 | 0.5 | 0.6 |
| 34 | 1.0 | 0.6 | 0.6 |
| 36 | 1.0 | 0.7 | 0.6 |
| TOGA | 1.0 | 0.2 | 0.1 |

TOGA analysis showed that the expression of Rec1_{—}12 is substantially down-regulated at 8 weeks in the aorta of rabbits with fatty atherosclerotic lesion development caused by hyper-cholesterolemia. The expression pattern was verified in the RT-PCR study, which showed that the expression of Rec1_{—}12 at 8 weeks is decreased both in hypercholesterolemic animals and in hypercholesterolemic animals receiving lercanidipine.

### EXAMPLE 5

### Identification and Characterization of Polynucleotides Regulated by Lercanidipine in Aorta

TOGA analysis further identified several clones whose expression is affected by the administration of a racemic mixture of lercanidipine. For example, the expression REC1_{—}22 and REC1_{—}24 is down-regulated in the aorta of normal and hypercholesterolemic rabbits treated with lercanidipine compared with untreated aorta.

Clone REC1_{—}22 (digital address TGGG 164) was obtained using the above-described TOGA analysis methods. The TOGA data was generated with a 5′-PCR primer (C-G-A-C-G-G-T-A-T-C-G-G-T-G-G-G, SEQ ID NO: 78) labeled with 6-carboxyfluorescein (6FAM, ABI) at the 5′ terminus. PCR products were resolved by gel electrophoresis on 4.5% acrylamide gels and the fluorescence data acquired on ABI377 automated sequencers. Data were analyzed using GeneScan software.

As shown in Table 1, the results of TOGA analysis indicate that the expression of REC1_{—}22 is down-regulated by lercanidipine treatment. In normal day 0 aorta, the expression of REC1_{—}22 is greater (430) than the expression in lercanidipine-treated day 0 aorta (87). Similarly, at day 14 of cholesterol administration, the expression is greater in normal aorta (381) than in lercanidipine-treated aorta (194).

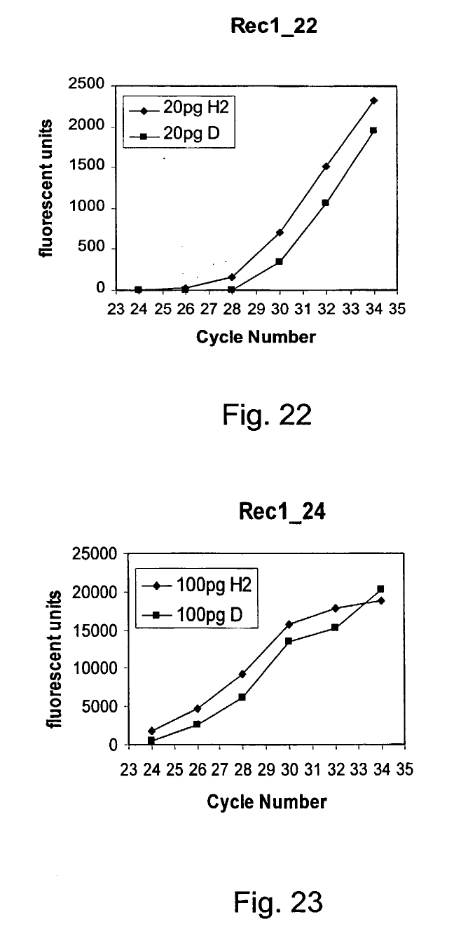

The results of the quantified RT-PCR for Rec1_{—}22 are shown in FIG. 22 and in Table 21. FIG. 22 is a graphical representation of the results of RT-PCR using 20 pg of clone REC1_{—}22 template, in which the amount of PCR product (measured in arbitrary fluorescence units) is plotted against number of cycles, where polyA enriched mRNA was extracted from aortas of rabbits that were normocholesterolemic (filled diamonds, “H2”) and hypercholesterolemic plus lercanidipine R (−), eight weeks (filled squares, “D”).

TABLE 21
Relative Abundance Rec1_22
20 pg template

| Cycle | 20 pg H2 | 20 pg D |
|---|---|---|
| 26 | 1.0 | 0.0 |
| 28 | 1.0 | 0.0 |
| 30 | 1.0 | 0.5 |
| 32 | 1.0 | 0.7 |
| 34 | 1.0 | 0.8 |
| TOGA | 1.0 | 0.1 |

TOGA analysis showed that the expression of Rec1_{—}22 is substantially down-regulated at 8 weeks in the aorta of rabbits with fatty atherosclerotic lesion development caused by hyper-cholesterolemia. The expression pattern was verified in the RT-PCR study, which showed that the expression of Rec1_{—}22 at 8 weeks is decreased in hypercholesterolemic animals receiving lercanidipine R (−) compared to normocholesterolemic animals.

Clone REC1_{—}24 (digital address CCGG 232) was obtained using the above-described TOGA analysis methods. The TOGA data was generated with a 5′-PCR primer (C-G-A-C-G-G-T-A-T-C-G-G-C-C-G-G, SEQ ID NO: 79) labeled with 6-carboxyfluorescein (6FAM, ABI) at the 5′ terminus. Table 1 indicates that the expression of REC1_{—}24 is down-regulated by lercanidipine treatment. In normal day 0 aorta, the expression of REC1_{—}24 is greater (449) than the expression in lercanidipine-treated day 0 aorta (122). Similarly, at day 14 of cholesterol diet, the expression is greater in normal aorta (273) than in lercanidipine-treated aorta (104).

The results of the quantified RT-PCR for Rec1_{—}24 are shown in FIG. 23 and in Table 22. FIG. 23 is a graphical representation of the results of RT-PCR using 100 pg of clone REC1_{—}24 template, in which the amount of PCR product (measured in arbitrary fluorescence units) is plotted against number of cycles, where polyA enriched mRNA was extracted from aortas of rabbits that were normocholesterolemic (filled diamonds, “H2”) and hypercholesterolemic plus lercanidipine R (−), eight weeks (filled squares, “D”).

TABLE 22
Relative Abundance Rec1_24
100 pg template

| Cycle | 100 pg H2 | 100 pg D |
|---|---|---|
| 24 | 1.0 | 0.3 |
| 26 | 1.0 | 0.5 |
| 28 | 1.0 | 0.7 |
| 30 | 1.0 | 0.9 |
| 32 | 1.0 | 0.9 |
| 34 | 1.0 | 1.1 |
| TOGA | 1.0 | 0.3 |

TOGA analysis showed that the expression of Rec1_{—}24 is substantially down-regulated at 8 weeks in the aorta of rabbits with fatty atherosclerotic lesion development caused by hyper-cholesterolemia. The expression pattern was verified in the RT-PCR study, which showed that the expression of Rec1_{—}24 at 8 weeks is generally somewhat decreased in hypercholesterolemic animals receiving lercanidipine R (−) compared to normocholesterolemic animals.

### EXAMPLE 6

### Identification and Characterization of Polynucleotides Differentially Regulated by Lercanidipine and (R)-Lercanidipine in Aorta

TOGA analysis further identified several clones whose expression is affected by the administration of lercanidipine, but not (R)-lercanidipine. As discussed previously, lercanidipine has a chiral center that produces two enantiomers. Since the (R)-enantiomer is approximately 2-3 orders of magnitude less effective as a ligand to the calcium channel, comparing the level of gene expression induced or suppressed by the (R)-enantiomer with the level induced or suppressed by the racemic mixture is useful to evaluate whether calcium antagonism plays a role in the anti-atherosclerotic activity of lercanidipine. Gene expression modulated by both the racemate and the (R)-enantiomer suggest that the modulation may not involve calcium channels. In contrast, gene expression induced or suppressed by the racemic mixture, but not the (R)-enantiomer, suggests that the differential expression may be due to calcium antagonist activity.

For example, Table 1 shows that the expression of REC1_{—}28 is down-regulated in the aorta of rabbits treated with the racemic form of lercanidipine, but not with the (R)-enantiomer of lercanidipine. Clone REC1_{—}28 (digital address CGGT 101) was obtained using the above-described TOGA analysis methods. As shown in Table 1, the results of TOGA analysis indicate that the expression of REC1_{—}28 is down-regulated by lercanidipine treatment. At day 0, the expression of REC1_{—}28 is greater in control aorta (1028) than in lercanidipine-treated aorta (229). Similarly, at day 14 of cholesterol diet, the expression is greater in control aorta (516) than in lercanidipine-treated aorta (189). However, the expression of REC1_{—}28 is not down-regulated in the aorta of hypercholesterolemic rabbits treated with (R)-lercanidipine, suggesting that the observed down-regulation with lercanidipine treatment may involve calcium antagonist activity.

In contrast, the expression of clones REC1_{—}31, REC1_{—}33, and REC1_{—}34 are up-regulated in the aorta of normal and hypercholesterolemic rabbits treated with lercanidipine compared with untreated aorta. However, the up-regulation is not observed in the aorta of rabbits treated with (R)-lercanidipine. Clones REC1_{—}31 (digital address TGCA 210), REC1_{—}33 (digital address CCGA 96), and REC1_{—}34 (digital address CGGT 209) were obtained using the above-described TOGA analysis methods. As shown in Table 1, the results of TOGA analysis indicate that the expression of REC1_{—}31 is up-regulated by lercanidipine treatment. At day 0, the expression of REC1_{—}31 in control aorta is lower (237) than the expression in lercanidipine-treated aorta (464). Similarly, at day 14 of cholesterol administration, the expression is lower in control aorta (134) than in lercanidipine-treated aorta (619) but not in (R)-lercanidipine-treated aorta (28). These results suggest that the up-regulation of REC1_{—}31 observed with lercanidipine treatment may involve calcium antagonist activity.

Similarly, at day 0, the expression of clone REC1_{—}33 is lower in control aorta (282) than in lercanidipine-treated aorta (1094). At day 14 of cholesterol administration, the expression is also lower in control aorta than in lercanidipine-treated aorta (842), but not in (R)-lercanidipine-treated aorta (170), suggesting that the up-regulation of REC1_{—}33 may involve calcium antagonist activity.

Also, at day 0, the expression of clone REC1_{—}34 is lower in control aorta (20) than in lercanidipine-treated aorta (100) and at day 14 is lower in control aorta (40) than in lercanidipine-treated aorta (331) but not in (R)-lercanidipine-treated aorta (58). Likewise, the data suggests the up-regulation of REC1_{—}34 observed with lercanidipine treatment involves calcium antagonist activity.

The above DST clones and at least part of the longer molecule corresponding to the DST clones can be useful in diagnosing or monitoring the effects of treating a subject with a dihydropyridine calcium antagonist. In one embodiment, the invention provides a method of diagnosing or montoring the effects of treating a subject with a dihydropyridine calcium antagonist comprising detecting an alteration in expression of at least one polypeptide encoded by at least one polynucleotide selected from the group consisting of SEQ ID NO:19, SEQ ID NO:20, SEQ ID NO:21, SEQ ID NO:22, SEQ ID NO:23 and SEQ ID NO:24.

### EXAMPLE 7

### Identification and Characterization of Polynucleotides Which Cholesterol Effect is Reversed by Lercanidimine in Aorta

In addition, TOGA analysis identified clones whose expression affected by the development of atherosclerotic fatty lesions is reversed by the treatment of lercanidipine. For example, the expression REC1_{—}36 is down-regulated during fatty lesion development. However, treatment with lercanidipine reverses the observed down-regulation in REC1_{—}36 expression. In contrast, the expression of clone REC1_{—}37 is up-regulated during fatty lesion development, which up-regulation is reversed by lercanidipine treatment.

Clones REC1_{—}36 (digital address TTAG 155) and REC1_{—}37 (digital address TTCA 264) were obtained using the above-described TOGA analysis methods. As shown in Table 1, the results of TOGA analysis indicate that the expression of REC1_{—}36 is down-regulated by fatty lesion development in untreated aorta. At day 0, the expression of REC1_{—}36 in control aorta (no cholesterol, no lercanidipine) is higher (736) than the expression in control aorta exposed to cholesterol for 14 days (104) and 8 weeks (206). However, this down-regulation is partially reversed with lercanidipine treatment. At day 14 of cholesterol administration, the expression of clone REC1_{—}36 was increased in aorta treated with lercanidipine (683) compared to control aorta (104). Similarly, at week 8 of cholesterol administration, the expression of clone REC1_{—}36 was increased in aorta treated with lercanidipine (400) compared to control aorta (206). Thus, the down-regulation of this clone induced by fatty lesion development is partially reversed with lercanidine treatment.

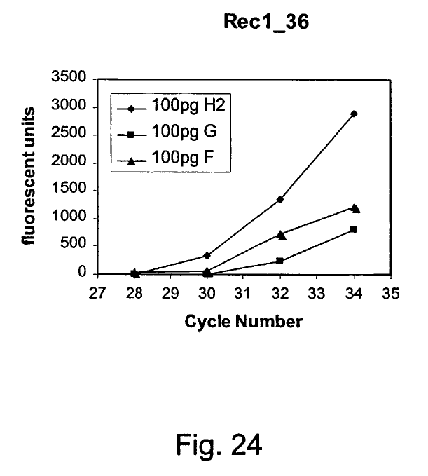

The results of the quantified RT-PCR for Rec1_{—}36 are shown in FIG. 24 and in Table 23. FIG. 24 is a graphical representation of the results of RT-PCR using 100 pg of clone REC1_{—}36 template, in which the amount of PCR product (measured in arbitrary fluorescence units) is plotted against number of cycles, where polyA enriched mRNA was extracted from aortas of rabbits that were normocholesterolemic (filled diamonds, “H2”), hypercholesterolemic, eight weeks (filled squares, “G”) and hypercholesterolemic plus lercanidipine racemate, eight weeks (filled triangles, “F”).

TABLE 23
Relative Abundance Rec1_36
100 pg template

| Cycle | 100 pg H2 | 100 pg G | 100 pg F |
|---|---|---|---|
| 30 | 1.0 | 0.0 | 0.2 |
| 32 | 1.0 | 0.2 | 0.5 |
| 34 | 1.0 | 0.3 | 0.4 |
| TOGA | 1.0 | 0.3 | 0.5 |

The results in Table 1 show that the expression of REC1137 is up-regulated by fatty lesion development in untreated aorta. At day 0, the expression of REC1_{—}37 in control aorta (no cholesterol, no lercanidipine) is lower (722) than the expression in control aorta exposed to cholesterol for 14 days (870) and 8 weeks (1462). At day 14, the observed up-regulation in expression (870) is not affected by lercanidipine treatment (915), but is affected by treatment with the (R)-enantiomer of lercanidipine (42). In addition, at week 8, the up-regulation (1462) is reversed by lercanidipine treatment (136). These results suggest that the up-regulation of REC1_{—}37 induced by fatty lesion development may be reversed by lercanidipine early on via an alternative mechanism that does not involve calcium channel blockage. At week 8, the effect of lercanidipine on REC1_{—}37 up-regulation may be mediated, in part, through calcium antagonist activity.

TABLE 1

| | | | | | | | | 14 Day | | |
|---|---|---|---|---|---|---|---|---|---|---|
| | | | | | | | 14 Day | hyper- | | 8 Week |
| | | Digital | | | | 14 Day | hyper- | cholesterol + | 8 Week | hyper- |
| Seq | | Address | | | Normal + | hyper- | cholesterol + | Lercanidipine | hyper- | cholesterol + |
| ID | Clone ID | (Msp1) | | Normal | Lercanidipine | cholesterol | Lercanidipine | (R) | cholesterol | Lercanidipine |
| | | AAAC | 187 | 83 | 313 | 271 | 338 | 295 | 537 | 347 |
| | | AACC | 86 | 389 | 289 | 804 | 146 | 357 | 448 | 489 |
| | | AACG | 94 | 159 | 324 | 582 | 73 | 332 | 518 | 465 |
| | | AACT | 130 | 647 | 487 | 334 | 574 | 156 | 591 | 504 |
| | | AAGC | 125 | 97 | 48 | 170 | 291 | 95 | 101 | 71 |
| 1 | REC1_1 | AAGC | 288 | 251 | 215 | 1008 | 753 | 872 | 1673 | 1733 |
| | | AAGC | 460 | 1252 | 1001 | 958 | 904 | 951 | 247 | 218 |
| | | AAGG | 105 | 498 | 274 | 522 | 127 | 392 | 205 | 252 |
| | | AAGG | 146 | 322 | 384 | 265 | 636 | 156 | 202 | 224 |
| | | AAGG | 205 | 346 | 42 | 646 | 233 | 53 | 112 | 98 |
| | | AATA | 88 | 489 | 104 | 296 | 387 | 113 | 762 | 1140 |
| | | AATA | 141 | 552 | 310 | 362 | 160 | 114 | 155 | 117 |
| | | AATA | 234 | 79 | 193 | 606 | 398 | 269 | 321 | 394 |
| 2 | REC1_2 | AATC | 108 | 177 | 301 | 791 | 545 | 1250 | 917 | 1143 |
| | | AATC | 141 | 656 | 454 | 385 | 278 | 658 | 222 | 193 |
| | | AATC | 191 | 187 | 142 | 87 | 487 | 148 | 337 | 407 |
| | | AATC | 208 | 459 | 495 | 995 | 302 | 265 | 572 | 418 |
| | | AATC | 227 | 363 | 223 | 225 | 220 | 294 | 106 | 104 |
| | | AATC | 367 | 149 | 89 | 74 | 302 | 168 | 129 | 203 |
| | | AATG | 211 | 82 | 175 | 73 | 305 | 55 | 217 | 119 |
| | | ACAA | 168 | 243 | 135 | 135 | 114 | 669 | 133 | 128 |
| | | ACAA | 212 | 535 | 585 | 1632 | 108 | 203 | 271 | 469 |
| | | ACAA | 240 | 357 | 414 | 652 | 185 | 205 | 680 | 477 |
| | | ACAA | 260 | 68 | 75 | 54 | 55 | 376 | 39 | 34 |
| | | ACAA | 265 | 54 | 82 | 13 | 304 | 60 | 63 | 88 |
| | | ACAA | 336 | 1055 | 194 | 272 | 235 | 508 | 188 | 131 |
| | | ACAA | 356 | 326 | 334 | 382 | 929 | 182 | 173 | 383 |
| | | ACAC | 170 | 733 | 1047 | 698 | 757 | 1027 | 2256 | 1806 |
| | | ACAC | 215 | 841 | 138 | 277 | 218 | 215 | 28 | 33 |
| 35 | REC1_42 | ACAC | 268 | 129 | 139 | 324 | 24 | 405 | 224 | 467 |
| | | ACAT | 91 | 737 | 1101 | 429 | 366 | 626 | 60 | 127 |
| | | ACAT | 117 | 1180 | 1594 | 906 | 4078 | 1914 | 6524 | 6880 |
| | | ACAT | 230 | 465 | 578 | 136 | 185 | 78 | 415 | 289 |
| | | ACAT | 252 | 605 | 622 | 353 | 191 | 865 | 465 | 732 |
| | | ACCA | 119 | 230 | 1072 | 502 | 431 | 996 | 465 | 784 |
| | | ACCA | 127 | 1866 | 523 | 706 | 627 | 1582 | 1043 | 1440 |
| | | ACCA | 137 | 179 | 167 | 493 | 597 | 85 | 325 | 352 |
| | | ACCA | 156 | 1113 | 2951 | 446 | 4203 | 2246 | 3124 | 2731 |
| 54 | REC1_61 | ACCA | 174 | 4035 | 3959 | 4230 | 2574 | 2287 | 1131 | 962 |
| | | ACCA | 287 | 120 | 99 | 292 | 56 | 387 | 191 | 269 |
| | | ACCC | 78 | 167 | 377 | 788 | 623 | 263 | 433 | 386 |
| | | ACCC | 148 | 1557 | 1573 | 2827 | 1373 | 1091 | 5906 | 6061 |
| | | ACCC | 177 | 478 | 318 | 218 | 1389 | 385 | 546 | 569 |
| | | ACCC | 219 | 623 | 738 | 576 | 1090 | 295 | 488 | 452 |
| | | ACCC | 389 | 211 | 344 | 807 | 1133 | 436 | 418 | 437 |
| | | ACCG | 83 | 96 | 40 | 100 | 293 | 61 | 364 | 68 |
| | | ACCG | 92 | 183 | 107 | 29 | 1254 | 25 | 288 | 121 |
| | | ACCG | 142 | 164 | 41 | 544 | 1073 | 363 | 985 | 1142 |
| | | ACCG | 295 | 44 | 96 | 87 | 647 | 5 | 49 | 75 |
| | | ACCT | 112 | 283 | 419 | 322 | 2228 | 497 | 1261 | 1076 |
| | | ACCT | 123 | 382 | 230 | 104 | 173 | 164 | 708 | 1057 |
| | | ACCT | 143 | 3710 | 2353 | 3395 | 1234 | 1867 | 767 | 796 |
| | | ACCT | 219 | 80 | 28 | 31 | 34 | 349 | 58 | 102 |
| | | ACCT | 273 | 221 | 207 | 21 | 56 | 28 | 141 | 121 |
| | | ACGA | 86 | 131 | 105 | 424 | 154 | 450 | 230 | 222 |
| | | ACGC | 144 | 283 | 243 | 287 | 417 | 338 | 1108 | 991 |
| | | ACGG | 79 | 139 | 124 | 1098 | 94 | 132 | 81 | 118 |
| 3 | REC1_3 | ACGG | 162 | 80 | 54 | 688 | 68 | 197 | 53 | 65 |
| | | ACTC | 82 | 66 | 173 | 112 | 187 | 726 | 164 | 243 |
| | | ACTC | 142 | 403 | 813 | 112 | 580 | 483 | 359 | 454 |
| | | ACTC | 154 | 102 | 89 | 214 | 79 | 107 | 132 | 501 |
| | | ACTC | 250 | 88 | 143 | 131 | 71 | 197 | 675 | 515 |
| | | ACTC | 292 | 699 | 396 | 347 | 390 | 389 | 151 | 181 |
| | | ACTC | 344 | 452 | 215 | 236 | 503 | 390 | 210 | 115 |
| | | ACTC | 365 | 246 | 461 | 275 | 41 | 491 | 732 | 1340 |
| | | ACTG | 212 | 1233 | 1239 | 1584 | 1099 | 1123 | 1301 | 1352 |
| | | ACTT | 203 | 402 | 234 | 99 | 167 | 333 | 87 | 140 |
| | | AGAG | 210 | 44 | 82 | 53 | 270 | 62 | 139 | 177 |
| | | AGAG | 222 | 1457 | 793 | 739 | 435 | 899 | 648 | 358 |
| | | AGAT | 112 | 1003 | 907 | 1090 | 350 | 912 | 772 | 674 |
| | | AGCA | 92 | 377 | 181 | 498 | 757 | 667 | 1739 | 1605 |
| | | AGCA | 160 | 551 | 187 | 470 | 81 | 297 | 153 | 462 |
| | | AGCA | 186 | 111 | 170 | 71 | 579 | 496 | 203 | 123 |
| | | AGCA | 196 | 346 | 297 | 310 | 155 | 607 | 540 | 722 |
| | | AGCA | 212 | 452 | 409 | 281 | 883 | 148 | 1128 | 1864 |
| | | AGCA | 285 | 855 | 228 | 375 | 235 | 315 | 642 | 493 |
| | | AGCC | 175 | 2665 | 2716 | 2186 | 2258 | 2392 | 6914 | 4221 |
| | | AGCC | 179 | 1939 | 2716 | 2142 | 2258 | 2392 | 6920 | 4221 |
| | | AGCC | 280 | 351 | 62 | 65 | 38 | 87 | 53 | 85 |
| | | AGCG | 90 | 901 | 186 | 243 | 1540 | 270 | 379 | 446 |
| | | AGCG | 237 | 67 | 15 | 78 | 529 | 52 | 79 | 31 |
| | | AGCG | 278 | 60 | 270 | 210 | 553 | 157 | 81 | 125 |
| | | AGCG | 295 | 316 | 73 | 248 | 218 | 110 | 982 | 1011 |
| | | AGCT | 121 | 282 | 392 | 56 | 1148 | 118 | 188 | 285 |
| | | AGCT | 225 | 106 | 93 | 1701 | 520 | 194 | 250 | 224 |
| | | AGCT | 328 | 194 | 138 | 570 | 301 | 120 | 104 | 148 |
| | | AGGA | 131 | 207 | 321 | 507 | 355 | 960 | 140 | 203 |
| | | AGGA | 139 | 2167 | 1911 | 1092 | 682 | 1771 | 1933 | 1822 |
| | | AGGA | 157 | 267 | 134 | 400 | 38 | 91 | 88 | 100 |
| 43 | REC1_50 | AGGA | 205 | 1558 | 772 | 1099 | 868 | 816 | 499 | 379 |
| 36 | REC1_43 | AGGC | 164 | 284 | 268 | 496 | 798 | 535 | 971 | 1172 |
| | | AGGC | 223 | 835 | 876 | 212 | 641 | 716 | 810 | 753 |
| | | AGGG | 125 | 537 | 247 | 186 | 552 | 492 | 520 | 671 |
| | | AGGG | 176 | 1309 | 1042 | 526 | 1002 | 1218 | 135 | 98 |
| | | AGGG | 227 | 78 | 111 | 295 | 274 | 375 | 82 | 112 |
| | | AGGT | 120 | 344 | 513 | 598 | 161 | 284 | 115 | 85 |
| 37 | REC1_44 | AGGT | 157 | 231 | 258 | 1195 | 220 | 706 | 2317 | 2025 |
| | | AGGT | 168 | 580 | 587 | 116 | 171 | 147 | 204 | 287 |
| | | AGGT | 223 | 120 | 108 | 139 | 97 | 385 | 107 | 39 |
| 27 | REC1_4 | AGTA | 147 | 1413 | 1450 | 864 | 1219 | 1431 | 147 | 201 |
| | | AGTA | 228 | 483 | 308 | 175 | 113 | 403 | 290 | 232 |
| | | AGTC | 166 | 3290 | 3085 | 2333 | 1238 | 2857 | 1037 | 772 |
| | | AGTC | 219 | 263 | 167 | 278 | 527 | 294 | 1178 | 1194 |
| | | AGTG | 110 | 305 | 691 | 470 | 157 | 220 | 170 | 1250 |
| 4 | REC1_5 | AGTG | 184 | 1086 | 1034 | 433 | 699 | 643 | 285 | 188 |
| 51 | REC1_58 | AGTT | 124 | 2410 | 2143 | 1682 | 1975 | 1907 | 518 | 664 |
| | | AGTT | 136 | 213 | 203 | 155 | 653 | 144 | 315 | 407 |
| | | ATAA | 114 | 144 | 220 | 1005 | 324 | 210 | 99 | 326 |
| | | ATAC | 176 | 191 | 290 | 441 | 181 | 217 | 521 | 311 |
| | | ATAT | 94 | 245 | 86 | 607 | 42 | 1550 | 38 | 47 |
| | | ATAT | 102 | 411 | 275 | 130 | 717 | 153 | 271 | 484 |
| | | ATAT | 156 | 347 | 95 | 457 | 127 | 204 | 75 | 96 |
| | | ATAT | 188 | 496 | 1143 | 1181 | 1660 | 1433 | 2338 | 2750 |
| | | ATAT | 209 | 56 | 51 | 321 | 27 | 8 | 26 | 66 |
| | | ATAT | 215 | 104 | 183 | 228 | 322 | 67 | 29 | 27 |
| | | ATCA | 160 | 447 | 749 | 575 | 859 | 383 | 2625 | 1477 |
| | | ATCA | 263 | 61 | 215 | 72 | 354 | 176 | 129 | 124 |
| | | ATCC | 164 | 1302 | 419 | 586 | 147 | 732 | 1116 | 944 |
| 5 | REC1_6 | ATCC | 176 | 148 | 120 | 303 | 399 | 365 | 1020 | 1302 |
| | | ATCC | 214 | 485 | 472 | 295 | 376 | 122 | 213 | 160 |
| | | ATCC | 359 | 1000 | 798 | 702 | 583 | 738 | 247 | 155 |
| | | ATCG | 190 | 1793 | 526 | 703 | 376 | 610 | 437 | 591 |
| | | ATCG | 216 | 116 | 76 | 707 | 325 | 13 | 308 | 202 |
| | | ATTA | 105 | 96 | 344 | 113 | 202 | 97 | 420 | 308 |
| 52 | REC1_59 | ATTA | 147 | 2034 | 2200 | 1609 | 1095 | 2023 | 562 | 582 |
| 48 | REC1_55 | ATTT | 289 | 830 | 1194 | 957 | 1580 | 775 | 267 | 172 |
| | | CAAA | 141 | 71 | 350 | 50 | 46 | 17 | 82 | 57 |
| | | CAAC | 81 | 683 | 54 | 74 | 105 | 552 | 544 | 505 |
| | | CAAC | 110 | 341 | 562 | 1659 | 815 | 281 | 179 | 153 |
| | | CAAC | 144 | 2161 | 1080 | 1114 | 427 | 298 | 1245 | 1385 |
| | | CAAC | 159 | 590 | 446 | 720 | 786 | 600 | 1928 | 1686 |
| | | CAAC | 167 | 419 | 144 | 252 | 2563 | 501 | 796 | 689 |
| | | CAAC | 180 | 676 | 673 | 385 | 188 | 550 | 556 | 581 |
| | | CAAC | 189 | 956 | 1171 | 2598 | 570 | 478 | 1502 | 1172 |
| | | CAAC | 224 | 84 | 35 | 237 | 881 | 99 | 227 | 428 |
| | | CAAC | 230 | 729 | 367 | 141 | 159 | 185 | 104 | 336 |
| | | CAAC | 251 | 72 | 55 | 127 | 471 | 23 | 119 | 134 |
| | | CAAC | 294 | 178 | 198 | 405 | 109 | 245 | 139 | 125 |
| | | CAAC | 405 | 71 | 109 | 89 | 292 | 89 | 91 | 75 |
| | | CAAC | 444 | 758 | 532 | 593 | 222 | 78 | 81 | 122 |
| | | CAAC | 449 | 468 | 400 | 402 | 211 | 99 | 85 | 103 |
| | | CAAT | 162 | 332 | 406 | 825 | 231 | 340 | 198 | 162 |
| | | CAAT | 273 | 232 | 538 | 190 | 794 | 115 | 577 | 300 |
| | | CACA | 88 | 6150 | 5543 | 6016 | 3392 | 5529 | 4638 | 5392 |
| 6 | REC1_7 | CACA | 282 | 976 | 863 | 787 | 417 | 683 | 199 | 159 |
| | | CACG | 184 | 655 | 652 | 353 | 498 | 453 | 328 | 255 |
| | | CACT | 178 | 1836 | 1058 | 1284 | 431 | 727 | 771 | 717 |
| | | CACT | 319 | 320 | 88 | 242 | 437 | 178 | 123 | 195 |
| | | CAGA | 106 | 88 | 145 | 308 | 546 | 235 | 156 | 246 |
| | | CAGA | 112 | 733 | 685 | 275 | 1009 | 1175 | 1178 | 996 |
| | | CAGC | 97 | 3410 | 3092 | 3283 | 622 | 3294 | 2571 | 2376 |
| | | CAGC | 144 | 829 | 960 | 935 | 1396 | 1761 | 2015 | 2875 |
| 38 | REC1_45 | CAGT | 143 | 383 | 323 | 470 | 705 | 638 | 1517 | 1541 |
| | | CAGT | 169 | 1403 | 1442 | 1021 | 743 | 1015 | 543 | 449 |
| | | CAGT | 170 | 1398 | 1384 | 1013 | 743 | 938 | 484 | 432 |
| | | CATA | 298 | 55 | 52 | 191 | 26 | 25 | 58 | 104 |
| | | CATG | 166 | 264 | 259 | 939 | 67 | 132 | 457 | 394 |
| | | CATG | 258 | 1303 | 2475 | 350 | 735 | 1214 | 742 | 658 |
| | | CATT | 109 | 100 | 533 | 650 | 111 | 26 | 141 | 162 |
| | | CCAA | 143 | 427 | 1210 | 194 | 402 | 445 | 960 | 1126 |
| | | CCAC | 180 | 460 | 483 | 116 | 261 | 284 | 91 | 41 |
| | | CCAC | 192 | 16 | 7 | 1773 | 110 | 30 | 18 | 8 |
| | | CCAG | 86 | 136 | 456 | 535 | 3555 | 200 | 475 | 228 |
| | | CCAG | 173 | 234 | 201 | 199 | 666 | 160 | 74 | 111 |
| | | CCAG | 232 | 870 | 641 | 590 | 200 | 607 | 344 | 310 |
| | | CCAG | 243 | 711 | 577 | 674 | 197 | 42 | 26 | 18 |
| | | CCAT | 110 | 391 | 269 | 104 | 331 | 344 | 281 | 306 |
| | | CCAT | 181 | 1174 | 788 | 1299 | 671 | 648 | 201 | 234 |
| | | CCAT | 249 | 347 | 358 | 216 | 77 | 199 | 135 | 130 |
| | | CCCA | 149 | 450 | 478 | 289 | 4423 | 661 | 264 | 159 |
| | | CCCA | 191 | 330 | 90 | 310 | 213 | 275 | 579 | 545 |
| 46 | REC1_53 | CCCC | 194 | 558 | 600 | 1034 | 449 | 991 | 872 | 433 |
| | | CCCG | 93 | 414 | 175 | 243 | 357 | 498 | 233 | 287 |
| 7 | REC1_8 | CCCG | 243 | 87 | 163 | 566 | 373 | 539 | 1370 | 1136 |
| | | CCCT | 166 | 586 | 233 | 482 | 143 | 393 | 263 | 166 |
| | | CCCT | 240 | 295 | 615 | 287 | 813 | 206 | 175 | 344 |
| 23 | REC1_33 | CCGA | 96 | 282 | 1094 | 114 | 842 | 170 | 419 | 156 |
| | | CCGA | 155 | 145 | 675 | 721 | 235 | 169 | 373 | 329 |
| | | CCGA | 216 | 818 | 636 | 332 | 407 | 353 | 169 | 129 |
| | | CCGA | 259 | 768 | 726 | 445 | 169 | 277 | 1287 | 1985 |
| | | CCGA | 260 | 770 | 812 | 459 | 170 | 303 | 1326 | 2082 |
| | | CCGA | 384 | 203 | 330 | 311 | 394 | 99 | 87 | 210 |
| | | CCGG | 195 | 44 | 60 | 85 | 314 | 140 | 348 | 232 |
| 20 | REC1_24 | CCGG | 232 | 449 | 122 | 273 | 104 | 139 | 177 | 117 |
| | | CCGG | 294 | 166 | 869 | 727 | 538 | 846 | 214 | 223 |
| | | CCGT | 110 | 16 | 17 | 197 | 434 | 284 | 167 | 75 |
| | | CCGT | 200 | 71 | 54 | 125 | 108 | 328 | 290 | 30 |
| | | CCTA | 76 | 1722 | 99 | 93 | 73 | 499 | 584 | 1534 |
| | | CCTA | 98 | 1993 | 4943 | 246 | 4667 | 400 | 3871 | 4587 |
| | | CCTA | 151 | 3017 | 2884 | 3486 | 292 | 1985 | 4405 | 5506 |
| | | CCTA | 209 | 1644 | 266 | 295 | 1296 | 538 | 606 | 279 |
| | | CCTA | 282 | 246 | 253 | 140 | 539 | 220 | 214 | 115 |
| | | CCTA | 288 | 221 | 755 | 91 | 186 | 102 | 202 | 269 |
| | | CCTC | 158 | 308 | 652 | 399 | 3956 | 3164 | 622 | 492 |
| | | CCTC | 226 | 186 | 83 | 304 | 160 | 232 | 555 | 812 |
| | | CCTG | 169 | 629 | 399 | 575 | 603 | 444 | 1351 | 1351 |
| | | CCTG | 172 | 300 | 136 | 284 | 129 | 173 | 647 | 523 |
| | | CCTT | 116 | 453 | 141 | 167 | 873 | 381 | 336 | 348 |
| | | CCTT | 254 | 906 | 553 | 252 | 401 | 749 | 342 | 481 |
| | | CGAA | 166 | 165 | 314 | 112 | 243 | 123 | 26 | 19 |
| | | CGAC | 85 | 424 | 498 | 999 | 120 | 887 | 271 | 433 |
| | | CGAG | 152 | 550 | 508 | 230 | 244 | 825 | 2161 | 2044 |
| | | CGAT | 262 | 979 | 1032 | 999 | 951 | 1166 | 242 | 266 |
| | | CGAT | 345 | 493 | 735 | 297 | 470 | 500 | 118 | 102 |
| | | CGCA | 101 | 497 | 518 | 1937 | 233 | 365 | 329 | 372 |
| | | CGCA | 149 | 144 | 180 | 88 | 2469 | 159 | 216 | 257 |
| | | CGCG | 138 | 184 | 248 | 1638 | 412 | 169 | 327 | 371 |
| | | CGCG | 145 | 307 | 315 | 221 | 470 | 263 | 447 | 601 |
| | | CGCG | 157 | 61 | 42 | 95 | 268 | 78 | 128 | 120 |
| | | CGCG | 192 | 268 | 460 | 226 | 94 | 374 | 302 | 337 |
| | | CGCT | 150 | 738 | 743 | 4142 | 534 | 570 | 611 | 601 |
| 39 | REC1_46 | CGCT | 219 | 432 | 367 | 552 | 385 | 668 | 1457 | 1787 |
| | | CGCT | 253 | 367 | 252 | 252 | 296 | 88 | 32 | 31 |
| | | CGGA | 289 | 439 | 19 | 154 | 59 | 86 | 25 | 42 |
| | | CGGA | 423 | 320 | 475 | 323 | 74 | 344 | 66 | 114 |
| | | CGGC | 212 | 357 | 725 | 243 | 2634 | 290 | 642 | 898 |
| | | CGGC | 260 | 544 | 126 | 287 | 10 | 234 | 55 | 81 |
| | | CGGG | 228 | 106 | 147 | 678 | 693 | 550 | 468 | 456 |
| 45 | REC1_52 | CGGG | 293 | 1747 | 677 | 893 | 469 | 617 | 151 | 257 |
| | | CGGT | 83 | 2469 | 816 | 2136 | 600 | 1941 | 418 | 632 |
| 21 | REC1_28 | CGGT | 101 | 1028 | 229 | 516 | 189 | 864 | 168 | 176 |
| | | CGGT | 112 | 77 | 100 | 30 | 695 | 245 | 522 | 409 |
| | | CGGT | 116 | 196 | 434 | 161 | 564 | 534 | 771 | 613 |
| 24 | REC1_34 | CGGT | 209 | 20 | 100 | 40 | 331 | 58 | 121 | 203 |
| | | CGGT | 277 | 417 | 244 | 2425 | 218 | 411 | 338 | 284 |
| | | CGTG | 143 | 212 | 252 | 109 | 433 | 195 | 310 | 432 |
| | | CGTG | 194 | 755 | 347 | 158 | 658 | 405 | 164 | 157 |
| | | CGTT | 106 | 67 | 429 | 323 | 112 | 334 | 463 | 346 |
| | | CTAA | 204 | 170 | 287 | 351 | 608 | 342 | 600 | 642 |
| | | CTAT | 90 | 173 | 104 | 895 | 255 | 667 | 353 | 275 |
| | | CTCC | 195 | 191 | 144 | 79 | 615 | 189 | 330 | 265 |
| | | CTCG | 152 | 203 | 299 | 268 | 466 | 290 | 565 | 843 |
| | | CTCT | 175 | 514 | 190 | 299 | 126 | 137 | 70 | 79 |
| | | CTGA | 144 | 160 | 234 | 250 | 838 | 164 | 1100 | 1130 |
| | | CTGA | 157 | 919 | 1258 | 340 | 764 | 597 | 1053 | 944 |
| | | CTGA | 311 | 497 | 338 | 303 | 168 | 262 | 111 | 126 |
| 8 | REC1_10 | CTGA | 390 | 54 | 45 | 802 | 670 | 906 | 293 | 399 |
| | | CTGC | 127 | 6054 | 5373 | 4121 | 6760 | 4482 | 2266 | 1864 |
| 34 | REC1_40 | CTGC | 130 | 5677 | 4608 | 3867 | 5781 | 4237 | 1808 | 1413 |
| | | CTGG | 293 | 455 | 419 | 271 | 289 | 388 | 117 | 99 |
| 33 | REC1_39 | CTGG | 429 | 1098 | 1384 | 1625 | 1452 | 2171 | 323 | 247 |
| | | CTTA | 95 | 495 | 587 | 159 | 2951 | 679 | 849 | 791 |
| | | CTTA | 136 | 240 | 239 | 221 | 1176 | 379 | 500 | 148 |
| | | CTTA | 161 | 465 | 290 | 1740 | 307 | 720 | 898 | 1031 |
| 31 | REC1_35 | CTTA | 195 | 165 | 399 | 41 | 382 | 78 | 103 | 230 |
| | | CTTA | 197 | 150 | 340 | 44 | 405 | 120 | 146 | 246 |
| | | CTTA | 405 | 196 | 130 | 92 | 472 | 107 | 215 | 284 |
| | | CTTC | 200 | 146 | 21 | 69 | 261 | 199 | 384 | 543 |
| | | CTTG | 169 | 292 | 480 | 511 | 88 | 85 | 335 | 474 |
| | | CTTG | 188 | 118 | 108 | 61 | 245 | 89 | 62 | 39 |
| | | CTTG | 251 | 330 | 146 | 48 | 291 | 9 | 80 | 55 |
| | | CTTT | 84 | 289 | 248 | 398 | 123 | 78 | 93 | 140 |
| | | CTTT | 231 | 111 | 48 | 265 | 45 | 64 | 100 | 37 |
| | | GAAA | 97 | 127 | 136 | 247 | 1191 | 199 | 232 | 168 |
| | | GAAC | 120 | 2240 | 1403 | 828 | 571 | 1366 | 1450 | 926 |
| | | GAAC | 285 | 250 | 339 | 413 | 433 | 687 | 754 | 933 |
| | | GAAT | 115 | 107 | 137 | 218 | 531 | 188 | 282 | 243 |
| | | GAAT | 225 | 449 | 262 | 124 | 475 | 441 | 297 | 322 |
| | | GAAT | 256 | 167 | 217 | 244 | 94 | 384 | 489 | 598 |
| | | GACA | 146 | 825 | 341 | 1164 | 278 | 1841 | 383 | 356 |
| | | GACC | 104 | 737 | 160 | 709 | 182 | 250 | 295 | 310 |
| | | GACC | 178 | 289 | 376 | 270 | 271 | 911 | 380 | 550 |
| | | GACC | 285 | 53 | 787 | 658 | 115 | 1426 | 107 | 118 |
| | | GACC | 377 | 540 | 38 | 58 | 82 | 70 | 253 | 260 |
| | | GACG | 96 | 1011 | 940 | 1015 | 137 | 692 | 1226 | 1501 |
| | | GACG | 155 | 666 | 634 | 386 | 1369 | 771 | 675 | 580 |
| | | GACG | 302 | 115 | 205 | 254 | 216 | 53 | 310 | 314 |
| 44 | REC1_51 | GACT | 89 | 271 | 216 | 451 | 269 | 244 | 853 | 876 |
| 55 | REC1_62 | GAGA | 168 | 3947 | 4060 | 2689 | 2574 | 2120 | 911 | 823 |
| | | GAGA | 223 | 573 | 528 | 458 | 166 | 238 | 158 | 106 |
| | | GAGC | 197 | 310 | 185 | 301 | 314 | 186 | 554 | 1025 |
| | | GATA | 95 | 287 | 459 | 2620 | 1130 | 441 | 791 | 1194 |
| | | GATA | 108 | 409 | 80 | 55 | 149 | 215 | 180 | 430 |
| | | GATA | 166 | 588 | 572 | 578 | 183 | 648 | 174 | 175 |
| | | GATA | 194 | 625 | 147 | 93 | 133 | 431 | 507 | 717 |
| 30 | REC1_32 | GATA | 202 | 97 | 252 | 118 | 258 | 97 | 699 | 786 |
| | | GATA | 225 | 138 | 133 | 128 | 41 | 551 | 187 | 124 |
| | | GATA | 250 | 58 | 65 | 74 | 450 | 57 | 86 | 52 |
| | | GATA | 300 | 676 | 736 | 744 | 93 | 591 | 289 | 79 |
| 40 | REC1_47 | GATC | 225 | 347 | 468 | 940 | 942 | 799 | 1176 | 1500 |
| | | GATC | 312 | 197 | 566 | 386 | 768 | 346 | 184 | 305 |
| | | GATG | 251 | 163 | 332 | 253 | 698 | 466 | 124 | 172 |
| | | GATT | 181 | 348 | 230 | 204 | 92 | 257 | 46 | 89 |
| | | GCAA | 80 | 287 | 670 | 250 | 4170 | 685 | 1131 | 1410 |
| | | GCAA | 84 | 465 | 605 | 422 | 564 | 816 | 1140 | 1474 |
| | | GCAA | 105 | 372 | 133 | 93 | 256 | 459 | 288 | 220 |
| | | GCAA | 125 | 660 | 332 | 604 | 75 | 351 | 274 | 185 |
| | | GCAA | 210 | 297 | 326 | 576 | 125 | 305 | 329 | 344 |
| | | GCAA | 244 | 220 | 377 | 62 | 93 | 374 | 145 | 66 |
| | | GCAA | 274 | 378 | 167 | 189 | 912 | 147 | 118 | 144 |
| | | GCAC | 126 | 6780 | 5598 | 3488 | 5264 | 394 | 4374 | 3927 |
| | | GCAG | 190 | 1058 | 1026 | 761 | 319 | 731 | 221 | 214 |
| | | GCAG | 271 | 528 | 703 | 1134 | 653 | 699 | 1705 | 2024 |
| 9 | REC1_11 | GCAT | 82 | 319 | 283 | 1351 | 778 | 1672 | 3663 | 3684 |
| | | GCCA | 116 | 583 | 766 | 283 | 1220 | 396 | 543 | 690 |
| | | GCCA | 188 | 191 | 258 | 145 | 642 | 162 | 187 | 296 |
| 11 | REC1_13 | GCCC | 216 | 705 | 575 | 509 | 542 | 404 | 4181 | 4186 |
| 10 | REC1_12 | GCCC | 232 | 5670 | 4929 | 4986 | 5657 | 3993 | 987 | 622 |
| | | GCCG | 96 | 1605 | 543 | 3049 | 312 | 2004 | 52 | 68 |
| | | GCCG | 159 | 601 | 466 | 103 | 1124 | 539 | 447 | 255 |
| | | GCGA | 101 | 1168 | 1212 | 769 | 413 | 2922 | 1478 | 1505 |
| | | GCGC | 124 | 247 | 392 | 601 | 900 | 204 | 322 | 390 |
| | | GCGC | 201 | 239 | 261 | 470 | 517 | 382 | 1383 | 1606 |
| | | GCGG | 188 | 1200 | 862 | 785 | 288 | 700 | 849 | 926 |
| | | GCGT | 101 | 1031 | 772 | 5290 | 533 | 4072 | 1566 | 1936 |
| | | GCGT | 140 | 311 | 497 | 605 | 913 | 104 | 1477 | 1240 |
| | | GCGT | 143 | 311 | 497 | 605 | 630 | 291 | 1380 | 1313 |
| | | GCGT | 159 | 642 | 1025 | 395 | 94 | 581 | 608 | 791 |
| | | GCGT | 169 | 91 | 119 | 17 | 342 | 226 | 563 | 148 |
| 53 | REC1_60 | GCGT | 178 | 2633 | 2299 | 1981 | 2285 | 1466 | 357 | 212 |
| | | GCTA | 220 | 1391 | 1244 | 995 | 1009 | 1053 | 344 | 394 |
| | | GCTC | 282 | 103 | 63 | 42 | 270 | 59 | 86 | 143 |
| | | GCTG | 302 | 53 | 237 | 67 | 96 | 99 | 69 | 85 |
| | | GGAA | 379 | 381 | 264 | 146 | 66 | 268 | 243 | 421 |
| | | GGAC | 86 | 279 | 85 | 433 | 1540 | 447 | 1624 | 1533 |
| | | GGAC | 225 | 1628 | 649 | 770 | 1113 | 698 | 388 | 426 |
| | | GGAT | 337 | 924 | 502 | 314 | 543 | 481 | 167 | 94 |
| | | GGCC | 87 | 94 | 150 | 107 | 83 | 3560 | 205 | 97 |
| | | GGCC | 232 | 616 | 766 | 411 | 644 | 863 | 97 | 125 |
| | | GGCC | 294 | 457 | 469 | 637 | 147 | 946 | 273 | 255 |
| | | GGCT | 84 | 211 | 204 | 136 | 716 | 229 | 914 | 1092 |
| | | GGCT | 150 | 451 | 269 | 565 | 329 | 382 | 75 | 64 |
| | | GGCT | 219 | 268 | 177 | 74 | 124 | 222 | 104 | 99 |
| | | GGCT | 397 | 403 | 411 | 418 | 444 | 355 | 81 | 88 |
| | | GGGA | 99 | 811 | 233 | 573 | 513 | 982 | 251 | 238 |
| | | GGGA | 167 | 96 | 411 | 173 | 20 | 76 | 75 | 63 |
| | | GGGA | 372 | 65 | 58 | 45 | 58 | 246 | 66 | 60 |
| | | GGGA | 474 | 66 | 68 | 320 | 68 | 219 | 58 | 74 |
| | | GGGC | 168 | 133 | 61 | 75 | 2940 | 207 | 76 | 110 |
| | | GGGG | 182 | 571 | 640 | 916 | 119 | 484 | 1112 | 1326 |
| | | GGGG | 204 | 58 | 230 | 34 | 55 | 26 | 104 | 80 |
| | | GGTA | 123 | 197 | 235 | 475 | 382 | 1830 | 435 | 258 |
| | | GGTA | 216 | 80 | 85 | 134 | 109 | 415 | 120 | 112 |
| | | GGTC | 229 | 200 | 96 | 271 | 64 | 125 | 66 | 68 |
| | | GGTG | 109 | 123 | 278 | 623 | 193 | 29 | 103 | 144 |
| | | GGTG | 144 | 510 | 843 | 798 | 2942 | 1030 | 796 | 777 |
| | | GGTG | 170 | 1294 | 1314 | 1184 | 366 | 521 | 1090 | 1019 |
| | | GGTG | 281 | 273 | 269 | 274 | 185 | 80 | 197 | 119 |
| | | GGTT | 190 | 248 | 267 | 496 | 203 | 414 | 789 | 838 |
| | | GTAA | 148 | 107 | 112 | 82 | 477 | 37 | 163 | 123 |
| | | GTAC | 225 | 461 | 284 | 83 | 270 | 143 | 113 | 135 |
| | | GTAG | 162 | 288 | 438 | 488 | 736 | 200 | 944 | 899 |
| | | GTAG | 272 | 120 | 243 | 403 | 117 | 208 | 282 | 498 |
| | | GTAT | 82 | 99 | 129 | 462 | 275 | 336 | 653 | 651 |
| | | GTCA | 182 | 528 | 211 | 290 | 109 | 182 | 238 | 177 |
| | | GTCC | 80 | 165 | 53 | 2181 | 1796 | 97 | 308 | 106 |
| | | GTCC | 111 | 2169 | 3440 | 1955 | 1317 | 2257 | 3631 | 4050 |
| | | GTCC | 204 | 87 | 502 | 311 | 87 | 155 | 661 | 503 |
| | | GTCC | 220 | 329 | 499 | 268 | 1960 | 215 | 962 | 690 |
| | | GTCC | 233 | 1627 | 302 | 989 | 1312 | 1012 | 61 | 76 |
| | | GTCC | 303 | 116 | 48 | 124 | 86 | 645 | 72 | 62 |
| | | GTGA | 168 | 1113 | 1638 | 674 | 545 | 571 | 324 | 157 |
| | | GTGA | 252 | 247 | 65 | 254 | 638 | 260 | 413 | 300 |
| 50 | REC1_57 | GTGA | 274 | 2143 | 1894 | 1151 | 1361 | 1407 | 577 | 1320 |
| 32 | REC1_38 | GTGC | 100 | 2625 | 2963 | 3402 | 2472 | 2498 | 672 | 857 |
| | | GTGC | 114 | 1853 | 1610 | 937 | 473 | 1075 | 2795 | 2719 |
| | | GTGC | 134 | 89 | 47 | 157 | 390 | 76 | 202 | 167 |
| | | GTGC | 229 | 92 | 289 | 69 | 199 | 272 | 490 | 623 |
| | | GTGC | 248 | 747 | 246 | 330 | 55 | 296 | 199 | 52 |
| | | GTGC | 307 | 193 | 257 | 225 | 378 | 107 | 379 | 308 |
| | | GTGG | 167 | 2787 | 1404 | 1328 | 1652 | 1211 | 444 | 309 |
| | | GTGG | 184 | 646 | 1251 | 1504 | 755 | 402 | 1025 | 906 |
| | | GTGG | 228 | 74 | 30 | 34 | 111 | 401 | 43 | 36 |
| | | GTGG | 436 | 86 | 172 | 109 | 743 | 130 | 60 | 43 |
| | | GTGT | 100 | 190 | 184 | 1782 | 184 | 1798 | 160 | 405 |
| | | GTTA | 115 | 280 | 111 | 183 | 744 | 125 | 529 | 482 |
| | | GTTC | 162 | 169 | 208 | 401 | 296 | 238 | 1346 | 2033 |
| | | GTTG | 116 | 107 | 234 | 282 | 327 | 195 | 506 | 289 |
| | | TAAA | 127 | 378 | 373 | 89 | 152 | 258 | 314 | 294 |
| | | TAAG | 137 | 172 | 24 | 50 | 524 | 133 | 190 | 116 |
| | | TAAG | 218 | 1298 | 605 | 2367 | 328 | 1029 | 365 | 359 |
| | | TAAT | 100 | 1208 | 899 | 706 | 309 | 725 | 573 | 640 |
| | | TAAT | 110 | 668 | 921 | 281 | 771 | 778 | 1155 | 228 |
| | | TAAT | 125 | 127 | 440 | 109 | 180 | 317 | 242 | 170 |
| | | TAAT | 133 | 78 | 238 | 41 | 358 | 479 | 328 | 241 |
| 29 | REC1_29 | TAAT | 162 | 157 | 233 | 85 | 572 | 30 | 142 | 258 |
| | | TAAT | 405 | 600 | 553 | 642 | 574 | 435 | 119 | 86 |
| | | TACC | 89 | 149 | 207 | 732 | 1062 | 310 | 748 | 864 |
| | | TACC | 97 | 681 | 2485 | 717 | 254 | 116 | 736 | 799 |
| | | TACC | 146 | 504 | 409 | 438 | 257 | 3566 | 379 | 529 |
| | | TACC | 178 | 3048 | 1307 | 3016 | 808 | 1515 | 79 | 236 |
| | | TACC | 197 | 198 | 290 | 554 | 594 | 394 | 1932 | 2159 |
| | | TACC | 214 | 829 | 536 | 527 | 548 | 562 | 163 | 68 |
| | | TACC | 259 | 327 | 92 | 191 | 212 | 51 | 234 | 255 |
| | | TACG | 103 | 81 | 639 | 78 | 105 | 1241 | 158 | 116 |
| | | TACG | 108 | 322 | 1951 | 582 | 155 | 297 | 1059 | 1276 |
| | | TACG | 138 | 1188 | 1172 | 485 | 1287 | 2594 | 711 | 705 |
| | | TACG | 150 | 1114 | 1129 | 2642 | 635 | 638 | 513 | 595 |
| | | TACG | 153 | 600 | 636 | 3801 | 358 | 345 | 286 | 416 |
| | | TACG | 187 | 295 | 255 | 118 | 2353 | 1877 | 688 | 1005 |
| | | TACG | 193 | 557 | 135 | 79 | 339 | 358 | 1007 | 746 |
| | | TACG | 197 | 172 | 318 | 38 | 307 | 343 | 645 | 406 |
| | | TACG | 247 | 132 | 432 | 327 | 82 | 40 | 153 | 31 |
| | | TACG | 261 | 117 | 67 | 276 | 727 | 98 | 38 | 35 |
| | | TACG | 264 | 106 | 97 | 528 | 410 | 43 | 46 | 63 |
| | | TACG | 298 | 165 | 57 | 27 | 159 | 895 | 382 | 180 |
| | | TACG | 429 | 126 | 142 | 248 | 100 | 62 | 37 | 41 |
| | | TACT | 116 | 187 | 345 | 308 | 978 | 566 | 169 | 150 |
| | | TACT | 122 | 116 | 481 | 121 | 453 | 423 | 822 | 663 |
| | | TACT | 138 | 277 | 281 | 787 | 117 | 237 | 181 | 154 |
| | | TACT | 156 | 539 | 78 | 298 | 53 | 134 | 94 | 238 |
| | | TACT | 289 | 379 | 66 | 165 | 75 | 147 | 22 | 44 |
| | | TAGA | 254 | 239 | 151 | 364 | 511 | 115 | 459 | 307 |
| | | TAGC | 145 | 1005 | 901 | 2372 | 911 | 516 | 1869 | 1298 |
| | | TAGC | 163 | 299 | 563 | 80 | 165 | 1054 | 958 | 1201 |
| 28 | REC1_25 | TAGC | 172 | 225 | 67 | 319 | 94 | 75 | 13 | 36 |
| | | TAGC | 229 | 990 | 720 | 594 | 869 | 752 | 513 | 261 |
| | | TAGC | 281 | 165 | 253 | 150 | 447 | 184 | 950 | 943 |
| | | TAGC | 325 | 340 | 117 | 279 | 127 | 571 | 247 | 129 |
| | | TAGC | 334 | 359 | 496 | 350 | 100 | 291 | 117 | 77 |
| | | TAGG | 141 | 1316 | 1445 | 1600 | 673 | 2985 | 2419 | 3024 |
| | | TAGG | 207 | 2580 | 1576 | 1329 | 767 | 1323 | 136 | 68 |
| | | TAGG | 265 | 84 | 68 | 75 | 59 | 489 | 286 | 129 |
| | | TAGT | 94 | 634 | 69 | 122 | 66 | 309 | 177 | 282 |
| | | TAGT | 146 | 180 | 411 | 82 | 152 | 605 | 401 | 328 |
| | | TAGT | 244 | 228 | 783 | 330 | 106 | 491 | 707 | 745 |
| | | TAGT | 264 | 286 | 74 | 130 | 122 | 576 | 30 | 103 |
| | | TATA | 96 | 1194 | 1229 | 1185 | 302 | 963 | 1407 | 1056 |
| | | TATC | 116 | 212 | 1048 | 79 | 173 | 37 | 290 | 274 |
| | | TATC | 119 | 454 | 667 | 266 | 413 | 80 | 512 | 504 |
| | | TATC | 132 | 264 | 281 | 867 | 566 | 2286 | 802 | 565 |
| | | TATC | 135 | 264 | 281 | 1904 | 566 | 1176 | 605 | 445 |
| | | TATC | 149 | 1443 | 701 | 183 | 641 | 466 | 226 | 351 |
| | | TATC | 163 | 164 | 33 | 2980 | 114 | 1833 | 201 | 274 |
| | | TATC | 180 | 2087 | 2184 | 170 | 892 | 623 | 1843 | 1253 |
| | | TATC | 215 | 220 | 67 | 1030 | 39 | 32 | 174 | 321 |
| | | TATC | 310 | 171 | 24 | 168 | 939 | 185 | 330 | 376 |
| | | TATC | 328 | 76 | 399 | 38 | 27 | 104 | 222 | 209 |
| | | TATG | 124 | 2490 | 1295 | 1981 | 1033 | 1490 | 822 | 983 |
| | | TATG | 145 | 380 | 44 | 25 | 199 | 1006 | 144 | 91 |
| | | TATG | 174 | 469 | 518 | 728 | 285 | 50 | 1568 | 963 |
| | | TATG | 179 | 311 | 307 | 238 | 1290 | 441 | 621 | 623 |
| | | TATT | 155 | 246 | 92 | 453 | 171 | 104 | 239 | 269 |
| | | TATT | 315 | 238 | 233 | 224 | 438 | 218 | 81 | 58 |
| | | TCAA | 162 | 218 | 891 | 163 | 783 | 347 | 273 | 169 |
| | | TCAA | 169 | 894 | 367 | 330 | 193 | 491 | 241 | 179 |
| | | TCAA | 181 | 202 | 475 | 57 | 425 | 65 | 182 | 585 |
| | | TCAA | 230 | 107 | 341 | 26 | 12 | 51 | 88 | 36 |
| | | TCAA | 235 | 78 | 184 | 33 | 113 | 286 | 286 | 75 |
| | | TCAA | 284 | 77 | 345 | 17 | 179 | 56 | 51 | 89 |
| | | TCAC | 92 | 1160 | 1119 | 965 | 320 | 945 | 2028 | 1834 |
| 12 | REC1_15 | TCAG | 76 | 904 | 1098 | 77 | 4118 | 278 | 356 | 444 |
| | | TCAG | 88 | 3167 | 1441 | 1974 | 1813 | 129 | 1110 | 560 |
| | | TCAG | 136 | 76 | 39 | 120 | 106 | 2237 | 186 | 157 |
| | | TCAG | 172 | 360 | 500 | 906 | 223 | 230 | 215 | 248 |
| | | TCAG | 269 | 427 | 116 | 252 | 157 | 267 | 149 | 63 |
| | | TCAG | 303 | 232 | 428 | 92 | 224 | 480 | 1141 | 1046 |
| | | TCAG | 316 | 105 | 148 | 589 | 188 | 323 | 249 | 304 |
| | | TCAT | 239 | 1716 | 553 | 674 | 613 | 488 | 166 | 268 |
| | | TCAT | 288 | 194 | 332 | 152 | 107 | 419 | 116 | 102 |
| | | TCCA | 79 | 617 | 155 | 75 | 216 | 641 | 431 | 361 |
| | | TCCA | 126 | 446 | 496 | 490 | 817 | 918 | 1744 | 1585 |
| | | TCCA | 135 | 354 | 427 | 626 | 232 | 129 | 475 | 370 |
| | | TCCA | 191 | 35 | 17 | 49 | 52 | 127 | 90 | 389 |
| | | TCCA | 233 | 144 | 75 | 36 | 768 | 132 | 36 | 169 |
| | | TCCA | 264 | 2768 | 1700 | 2193 | 1451 | 114 | 3720 | 893 |
| | | TCCA | 480 | 79 | 169 | 108 | 323 | 23 | 35 | 25 |
| | | TCCC | 140 | 573 | 78 | 196 | 402 | 107 | 129 | 217 |
| | | TCCG | 81 | 114 | 280 | 59 | 112 | 147 | 717 | 709 |
| | | TCCG | 86 | 146 | 1494 | 37 | 63 | 38 | 182 | 211 |
| | | TCCG | 93 | 589 | 252 | 89 | 1383 | 253 | 131 | 308 |
| 42 | REC1_49 | TCCG | 167 | 378 | 154 | 1584 | 6510 | 1080 | 297 | 224 |
| | | TCCG | 183 | 763 | 713 | 307 | 907 | 166 | 614 | 240 |
| | | TCCG | 186 | 1155 | 1077 | 307 | 692 | 101 | 894 | 354 |
| | | TCCG | 252 | 298 | 164 | 592 | 108 | 232 | 151 | 138 |
| | | TCCT | 154 | 165 | 385 | 291 | 1266 | 289 | 275 | 269 |
| | | TCCT | 211 | 834 | 539 | 629 | 436 | 304 | 393 | 185 |
| | | TCCT | 271 | 102 | 37 | 32 | 129 | 846 | 96 | 173 |
| | | TCGA | 129 | 115 | 119 | 396 | 111 | 812 | 178 | 130 |
| | | TCGA | 306 | 69 | 87 | 81 | 69 | 248 | 71 | 50 |
| | | TCGG | 84 | 120 | 484 | 35 | 287 | 50 | 295 | 86 |
| | | TCGG | 155 | 167 | 170 | 162 | 749 | 63 | 249 | 257 |
| 13 | REC1_16 | TCGG | 195 | 2497 | 2028 | 1247 | 1052 | 1554 | 479 | 530 |
| | | TCGG | 250 | 157 | 141 | 17 | 136 | 511 | 37 | 42 |
| | | TCGT | 84 | 462 | 266 | 957 | 449 | 923 | 127 | 271 |
| 14 | REC1_17 | TCGT | 199 | 1782 | 1434 | 832 | 713 | 1117 | 272 | 262 |
| | | TCTA | 111 | 2507 | 3950 | 615 | 2938 | 1673 | 2370 | 1904 |
| | | TCTA | 131 | 227 | 362 | 256 | 422 | 114 | 662 | 639 |
| | | TCTA | 150 | 589 | 510 | 1679 | 142 | 1450 | 409 | 418 |
| | | TCTA | 164 | 121 | 149 | 371 | 715 | 86 | 500 | 273 |
| | | TCTA | 167 | 74 | 131 | 755 | 474 | 101 | 288 | 138 |
| | | TCTA | 179 | 416 | 36 | 11 | 169 | 684 | 221 | 93 |
| | | TCTA | 194 | 163 | 283 | 16 | 44 | 24 | 403 | 55 |
| | | TCTA | 217 | 104 | 588 | 25 | 20 | 504 | 148 | 95 |
| | | TCTA | 228 | 1691 | 959 | 1024 | 684 | 1210 | 402 | 509 |
| | | TCTA | 264 | 891 | 800 | 659 | 380 | 55 | 750 | 466 |
| | | TCTA | 295 | 73 | 35 | 107 | 206 | 583 | 116 | 48 |
| | | TCTC | 152 | 114 | 274 | 303 | 1150 | 1319 | 568 | 405 |
| | | TCTC | 247 | 837 | 477 | 608 | 209 | 219 | 495 | 1078 |
| | | TCTC | 275 | 263 | 212 | 537 | 218 | 811 | 276 | 592 |
| | | TCTC | 419 | 444 | 517 | 501 | 467 | 289 | 104 | 107 |
| | | TCTG | 103 | 328 | 686 | 753 | 772 | 366 | 1599 | 1749 |
| | | TCTG | 115 | 593 | 776 | 981 | 238 | 684 | 1056 | 1052 |
| | | TCTG | 207 | 521 | 186 | 295 | 1089 | 855 | 607 | 532 |
| | | TCTT | 84 | 5835 | 94 | 3499 | 3032 | 3647 | 1836 | 2391 |
| | | TCTT | 354 | 173 | 541 | 521 | 470 | 706 | 587 | 608 |
| | | TGAA | 142 | 187 | 346 | 137 | 912 | 106 | 443 | 552 |
| | | TGAA | 187 | 1010 | 358 | 367 | 306 | 421 | 293 | 351 |
| | | TGAC | 94 | 345 | 360 | 275 | 267 | 940 | 307 | 338 |
| | | TGAC | 163 | 384 | 303 | 398 | 713 | 206 | 345 | 387 |
| | | TGAC | 191 | 281 | 100 | 44 | 1110 | 73 | 148 | 143 |
| 49 | REC1_56 | TGAC | 197 | 725 | 1212 | 849 | 744 | 645 | 163 | 130 |
| | | TGAC | 288 | 962 | 1214 | 584 | 77 | 603 | 349 | 599 |
| | | TGAC | 313 | 137 | 69 | 79 | 317 | 140 | 175 | 228 |
| | | TGAC | 343 | 57 | 159 | 115 | 35 | 334 | 90 | 96 |
| | | TGAC | 352 | 32 | 80 | 10 | 413 | 56 | 45 | 94 |
| | | TGAC | 392 | 66 | 80 | 114 | 288 | 63 | 64 | 57 |
| | | TGAG | 172 | 474 | 227 | 506 | 976 | 204 | 170 | 85 |
| | | TGAG | 184 | 135 | 137 | 79 | 99 | 277 | 268 | 468 |
| | | TGAG | 289 | 50 | 17 | 29 | 359 | 20 | 31 | 83 |
| | | TGAT | 124 | 532 | 625 | 1083 | 181 | 749 | 689 | 504 |
| | | TGAT | 163 | 108 | 359 | 181 | 721 | 71 | 158 | 121 |
| | | TGAT | 234 | 1231 | 467 | 102 | 324 | 314 | 708 | 624 |
| | | TGAT | 296 | 59 | 63 | 95 | 592 | 92 | 41 | 34 |
| | | TGCA | 174 | 92 | 222 | 110 | 212 | 400 | 298 | 258 |
| 22 | REC1_31 | TGCA | 210 | 237 | 464 | 134 | 619 | 28 | 122 | 245 |
| | | TGCA | 242 | 1745 | 1106 | 1051 | 630 | 938 | 321 | 216 |
| | | TGCA | 333 | 175 | 256 | 359 | 345 | 63 | 119 | 140 |
| | | TGCC | 185 | 316 | 133 | 140 | 283 | 121 | 1329 | 599 |
| | | TGCG | 78 | 1602 | 489 | 2897 | 286 | 2033 | 557 | 1025 |
| | | TGCG | 123 | 293 | 126 | 127 | 1033 | 565 | 246 | 149 |
| | | TGCG | 179 | 1263 | 282 | 129 | 834 | 101 | 43 | 23 |
| | | TGCG | 190 | 278 | 217 | 137 | 1035 | 54 | 217 | 253 |
| | | TGCG | 218 | 89 | 93 | 261 | 57 | 25 | 120 | 628 |
| | | TGCG | 232 | 37 | 156 | 169 | 451 | 37 | 190 | 68 |
| | | TGCG | 270 | 110 | 169 | 325 | 96 | 358 | 213 | 75 |
| | | TGCT | 187 | 124 | 150 | 103 | 960 | 26 | 306 | 307 |
| | | TGCT | 223 | 39 | 240 | 115 | 195 | 59 | 84 | 164 |
| | | TGCT | 235 | 900 | 610 | 131 | 927 | 1126 | 384 | 394 |
| 47 | REC1_54 | TGCT | 452 | 1107 | 1553 | 984 | 871 | 1130 | 118 | 108 |
| | | TGGA | 130 | 198 | 322 | 627 | 501 | 878 | 852 | 971 |
| | | TGGC | 122 | 396 | 261 | 742 | 260 | 5463 | 756 | 702 |
| | | TGGC | 173 | 231 | 577 | 100 | 22 | 474 | 121 | 185 |
| | | TGGC | 209 | 1416 | 425 | 673 | 442 | 310 | 847 | 1045 |
| | | TGGG | 76 | 886 | 643 | 339 | 416 | 1131 | 1771 | 1926 |
| | | TGGG | 116 | 692 | 758 | 1551 | 449 | 293 | 523 | 592 |
| | | TGGG | 137 | 2222 | 1338 | 1346 | 2831 | 490 | 808 | 786 |
| | | TGGG | 152 | 805 | 1382 | 987 | 997 | 1275 | 1708 | 3320 |
| 19 | REC1_22 | TGGG | 164 | 430 | 87 | 381 | 194 | 47 | 180 | 122 |
| | | TGGG | 218 | 428 | 475 | 86 | 96 | 308 | 171 | 93 |
| 15 | REC1_18 | TGGT | 241 | 188 | 316 | 224 | 112 | 330 | 799 | 935 |
| | | TGGT | 343 | 242 | 255 | 276 | 72 | 360 | 189 | 184 |
| | | TGGT | 383 | 365 | 357 | 337 | 640 | 340 | 78 | 92 |
| | | TGTA | 242 | 411 | 463 | 272 | 177 | 273 | 88 | 54 |
| | | TGTA | 314 | 167 | 222 | 153 | 880 | 156 | 264 | 171 |
| | | TGTC | 113 | 274 | 641 | 289 | 157 | 2536 | 481 | 487 |
| | | TGTC | 316 | 233 | 130 | 230 | 69 | 50 | 117 | 128 |
| | | TGTG | 137 | 1187 | 1000 | 928 | 2766 | 256 | 747 | 929 |
| | | TGTG | 238 | 629 | 681 | 412 | 71 | 572 | 204 | 161 |
| | | TGTG | 319 | 158 | 166 | 141 | 106 | 189 | 703 | 612 |
| | | TGTT | 158 | 140 | 531 | 221 | 544 | 520 | 417 | 588 |
| | | TGTT | 171 | 276 | 143 | 267 | 411 | 85 | 78 | 61 |
| | | TGTT | 178 | 99 | 56 | 378 | 60 | 96 | 41 | 68 |
| | | TGTT | 219 | 546 | 344 | 110 | 159 | 251 | 212 | 57 |
| | | TGTT | 350 | 54 | 251 | 35 | 15 | 43 | 107 | 142 |
| 16 | REC1_19 | TGTT | 393 | 687 | 569 | 322 | 373 | 748 | 96 | 130 |
| | | TTAA | 189 | 152 | 137 | 110 | 1168 | 153 | 118 | 159 |
| | | TTAA | 192 | 166 | 122 | 229 | 993 | 185 | 115 | 71 |
| | | TTAA | 217 | 196 | 254 | 86 | 29 | 444 | 103 | 370 |
| | | TTAA | 227 | 161 | 1592 | 602 | 36 | 108 | 112 | 99 |
| | | TTAA | 241 | 102 | 221 | 73 | 513 | 113 | 100 | 90 |
| | | TTAC | 79 | 805 | 464 | 341 | 180 | 1234 | 184 | 228 |
| | | TTAC | 93 | 45 | 63 | 806 | 305 | 1046 | 213 | 257 |
| | | TTAC | 149 | 1067 | 548 | 653 | 269 | 641 | 1049 | 1718 |
| 41 | REC1_48 | TTAC | 162 | 1567 | 1663 | 498 | 731 | 747 | 227 | 210 |
| 17 | REC1_20 | TTAC | 210 | 874 | 607 | 393 | 348 | 669 | 210 | 260 |
| | | TTAG | 78 | 394 | 1126 | 2010 | 448 | 733 | 293 | 201 |
| | | TTAG | 87 | 1040 | 1599 | 1817 | 1325 | 76 | 693 | 540 |
| 25 | REC1_36 | TTAG | 155 | 736 | 186 | 104 | 683 | 500 | 206 | 400 |
| | | TTAG | 177 | 29 | 61 | 79 | 72 | 871 | 34 | 135 |
| | | TTAG | 228 | 90 | 80 | 393 | 78 | 90 | 82 | 64 |
| | | TTAG | 299 | 86 | 56 | 82 | 54 | 389 | 183 | 135 |
| | | TTAG | 323 | 82 | 64 | 56 | 119 | 464 | 112 | 133 |
| | | TTAG | 404 | 147 | 218 | 526 | 73 | 283 | 34 | 27 |
| | | TTAT | 93 | 98 | 140 | 902 | 83 | 186 | 143 | 150 |
| | | TTAT | 111 | 767 | 1219 | 331 | 1157 | 997 | 1740 | 262 |
| | | TTCA | 132 | 1347 | 1361 | 952 | 268 | 689 | 456 | 316 |
| 26 | REC1_37 | TTCA | 264 | 722 | 605 | 870 | 915 | 42 | 1462 | 136 |
| 18 | REC1_21 | TTCC | 165 | 3144 | 2064 | 1203 | 1507 | 1574 | 636 | 486 |
| | | TTCC | 284 | 48 | 388 | 29 | 58 | 127 | 161 | 214 |
| | | TTCC | 308 | 54 | 41 | 227 | 122 | 91 | 72 | 83 |
| | | TTCG | 80 | 530 | 162 | 158 | 109 | 152 | 167 | 112 |
| | | TTCG | 93 | 186 | 61 | 69 | 167 | 719 | 555 | 404 |
| | | TTCG | 103 | 370 | 646 | 604 | 154 | 1037 | 357 | 409 |
| | | TTCG | 110 | 363 | 510 | 2666 | 135 | 1086 | 4555 | 4930 |
| | | TTCG | 113 | 363 | 512 | 1342 | 308 | 1455 | 3334 | 3058 |
| | | TTCG | 126 | 1720 | 750 | 2038 | 1789 | 1199 | 279 | 241 |
| | | TTCG | 139 | 136 | 248 | 173 | 435 | 70 | 253 | 550 |
| | | TTCG | 198 | 97 | 90 | 49 | 1280 | 207 | 125 | 459 |
| | | TTCG | 257 | 710 | 334 | 1761 | 143 | 259 | 368 | 241 |
| | | TTCT | 227 | 403 | 213 | 194 | 253 | 229 | 161 | 105 |
| | | TTGG | 276 | 473 | 296 | 221 | 649 | 287 | 57 | 97 |
| | | TTTC | 102 | 136 | 90 | 118 | 358 | 91 | 137 | 179 |
| | | TTTC | 428 | 340 | 421 | 699 | 316 | 450 | 76 | 123 |

TABLE 2

Nucleotide Homology

| | | | | | DST | |
|---|---|---|---|---|---|---|
| | | Digital | | | Nucleotide | Database |
| Seq | | Address | | | Range | Nucleotide |
| ID | Clone ID | (Msp1) | Database Match (Accession #) | % Homology | (bp#) | Range (bp#) |
| 1 | REC1_1 | AAGC 288 | Homo sapiens TYRO protein tyrosine kinase binding p | 95% | 13-109 | 317-413 |
| | | | mRNA, and translated products (NM_003332.1) | | | |
| 2 | REC1_2 | AATC 108 | NOVEL | N/A | N/A | N/A |
| 3 | REC1_3 | ACGG 162 | NOVEL | N/A | N/A | N/A |
| 4 | REC1_5 | AGTG 184 | NOVEL | N/A | N/A | N/A |
| 5 | REC1_6 | ATCC 176 | Rabbit mRNA for ferritin light chain subunit (X07830) | 99% | 1-121 | 637-757 |
| 6 | REC1_7 | CACA 282 | NOVEL | N/A | N/A | N/A |
| 7 | REC1_8 | CCCG 243 | NOVEL | N/A | N/A | N/A |
| 8 | REC1_10 | CTGA 390 | NOVEL | N/A | N/A | N/A |
| 9 | REC1_11 | GCAT 82 | NOVEL | N/A | N/A | N/A |
| 10 | REC1_12 | GCCC 232 | Cuniculus mRNA for alpha smooth muscle actin (X60732) | 99% | 1-176 | 1159-1332 |
| 11 | REC1_13 | GCCC 216 | NOVEL | N/A | N/A | N/A |
| 12 | REC1_15 | TCAG 76 | NOVEL | N/A | N/A | N/A |
| 13 | REC1_16 | TCGG 195 | NOVEL | N/A | N/A | N/A |
| 14 | REC1_17 | TCGT 199 | NOVEL | N/A | N/A | N/A |
| 15 | REC1_18 | TGGT 241 | NOVEL | N/A | N/A | N/A |
| 16 | REC1_19 | TGTT 393 | NOVEL | N/A | N/A | N/A |
| 17 | REC1_20 | TTAC 210 | Cuniculus phosphofructo-1-kinase C isozyme mRNA | 96% | 1-147 | 2594-2736 |
| | | | (U01154) | | | |
| 18 | REC1_21 | TTCC 165 | Human elastin gene, exon 1 (M17282) | 97% | 62-108 | 1028-1074 |
| 19 | REC1_22 | TGGG 164 | NOVEL | N/A | N/A | N/A |
| 20 | REC1_24 | CCGG 232 | NOVEL | N/A | N/A | N/A |
| 21 | REC1_28 | CGGT 101 | Rabbit mRNA for calgizzarin (D10586) | 96% | 1-29 | 1041-1069 |
| 22 | REC1_31 | TGCA 210 | Oryctolagus cuniculus complete mitochondrial genome | 100% | 4-153 | 8487-8636 |
| | | | (AJ001588) | | | |
| 23 | REC1_33 | CCGA 96 | Oryctolagus cuniculus as1_casein gene (M77195) | 97% | 8-45 | 7961-7998 |
| 24 | REC1_34 | CGGT 209 | NOVEL | N/A | N/A | N/A |
| 25 | REC1_36 | TTAG 155 | NOVEL | N/A | N/A | N/A |
| 26 | REC1_37 | TTCA 264 | R. norvegicus (Sprague Dawley) ribosomal protein L15 | 90% | 6-213 | 482-689 |
| | | | mRNA (X78167) | | | |
| 27 | REC1_4 | AGTA 147 | Homology to EST ut21b06.x1 Soares_thymus_2NbMT | 89% | 6-89 | 1-84 |
| | | | Mus musculus cDNA clone IMAGE: 3328499 3′ similar to | | | |
| | | | SW: RL2B_HUMAN P29316 60S RIBOSOMAL | | | |
| | | | PROTEIN L23A (BE691856.1) | | | |
| 28 | REC1_25 | TAGC 172 | Oryctolagus cuniculus ribosomal protein L5 homolog | 100% | 2-115 | 111-224 |
| | | | mRNA, partial cds (AF007880.1) | | | |
| 29 | REC1_29 | TAAT 162 | Homology to Homo sapiens SPARC-like 1 (mast9, hevin) | 89% | 4-109 | 2528-2634 |
| | | | (SPARCL1), mRNA (NM_004684.1) | | | |
| 30 | REC1_32 | GATA 202 | Oryctolagus cuniculus gene encoding ileal sodium- | 89% | 9-107 | 19089-19186 |
| | | | dependent bile acid transporter (AJ002005.1) | 86% | 11-107 | 25319-25414 |
| | | | | 93% | 60-107 | 24873-24919 |
| | | | | 83% | 11-71 | 21890-21985 |
| 31 | REC1_35 | CTTA 195 | Oryctolagus cuniculus mitochondrial cytB and tRNA-Thr | 100% | 2-138 | 1041-1177 |
| | | | genes for cytochrome b and transfer RNA-Thr | | | |
| | | | (AJ243197.1) | | | |
| 32 | REC1_38 | GTGC 100 | NOVEL | N/A | N/A | N/A |
| 33 | REC1_39 | CTGG 429 | Homologous to Mus musculus follistatin-like 2 (Fstl2) | 85% | 2-108 | 723-829 |
| | | | mRNA (NM_008048.1) | 84% | 291-361 | 1028-1103 |
| 34 | REC1_40 | CTGC 130 | NOVEL | N/A | N/A | N/A |
| 35 | REC1_42 | ACAC 268 | Homology to Human 18S rRNA gene (K03432.1) | 100% | 1-212 | 1414-1625 |
| 36 | REC1_43 | AGGC 164 | Homology to human ribosomal protein L13a (RPL13A) | 86% | 7-95 | 555-643 |
| | | | (NM_012423.1) | | | |
| 37 | REC1_44 | AGGT 157 | NOVEL | N/A | N/A | N/A |
| 38 | REC1_45 | CAGT 143 | Oryctolagus cuniculus cGMP-gated potassium channel | 90% | 7-47 | 645-685 |
| | | | (Kcnl) gene, 5UTR, promoter and enhancer (U38183.1) | | | |
| 39 | REC1_46 | CGCT 219 | Homology to Homo sapiens Arp2/3 protein complex | 83% | 86-160 | 1726-1811 |
| | | | subunit p16-Arc (ARC16) mRNA, complete cds | | | |
| | | | (AF006088.1) | | | |
| 40 | REC1_47 | GATC 225 | Homology to EST ia05c08.x1 Human Pancreatic Islets | 85% | 7-124 | 53-167 |
| | | | Homo sapiens cDNA 3 similar to gb: X62320 | | | |
| | | | GRANULINS PRECURSOR (HUMAN); gb: M86736 | | | |
| | | | Mouse acrogranin mRNA, complete cds (MOUSE) | | | |
| | | | (AW583467.1) | | | |
| 41 | REC1_48 | TTAC 162 | Homologous to Homo sapiens CGI-134 protein mRNA, | 84% | 3-102 | 423-522 |
| | | | complete cds. (AF151892.1) | | | |
| 42 | REC1_49 | TCCG 167 | Homology to Rat mRNA for ribosomal protein L7a | 86% | 7-65 | 746-804 |
| | | | (X15013.1) | | | |
| 43 | REC1_50 | AGGA 205 | Homology to human CGI-71 protein mRNA | 89% | 37-143 | 1907-2013 |
| | | | (AF151829.1) | | | |
| 44 | REC1_51 | GACT 89 | NOVEL | N/A | N/A | N/A |
| 45 | REC1_52 | CGGG 293 | Homology to Homo sapiens ribosomal protein S10 | 91% | 12-230 | 330-548 |
| | | | (RPS10) mRNA (NM_001014.1) | | | |
| 46 | REC1_53 | CCCC 194 | NOVEL | N/A | N/A | N/A |
| (ds) | | | | | | |
| 47 | REC1_54 | TGCT 452 | Homology to Homo sapiens mRNA for alpha-tropomyosin | 85% | 33-209 | 534-711 |
| | | | (3′ end) (AJ000147.1) | 87% | 281-401 | 774-890 |
| 48 | REC1_55 | ATTT 289 | Homology to Homo sapiens four and a half LIM domains | 85% | 2-230 | 1972-2203 |
| | | | 1 (FHL1), mRNA (XM_010369.1) | | | |
| 49 | REC1_56 | TGAC 197 | Homology to Homo sapiens ribosomal protein S23 | 89% | 2-98 | 350-446 |
| | | | (RPS23) mRNA (NM_001025.1) | | | |
| 50 | REC1_57 | GTGA 274 | Homology to Homo sapiens ribosomal protein L14 | 91% | 10-212 | 156-358 |
| | | | (RPL14) mRNA (NM_003973.1) | | | |
| 51 | REC1_58 | AGTT 124 | NOVEL | N/A | N/A | N/A |
| 52 | REC1_59 | ATTA 147 | Homology to Homo sapiens genomic DNA, chromosome | 93% | 3-80 | 252095-252172 |
| | | | 21q, section 68/105 (AP001724.1) | | | |
| 53 | REC1_60 | GCGT 178 | EST hv32a11.x1 NCI_CGAP_Lu24 Homo sapiens cDNA | 93% | 1-58 | 56-113 |
| | | | clone IMAGE: 3175100 (BE217941.1) | 96% | 95-127 | 2-34 |
| 54 | REC1_61 | ACCA 174 | NOVEL | N/A | N/A | N/A |
| 55 | REC1_62 | GAGA 168 | Homo sapiens zyxin (ZYX) mRNA (NM_003461.1) | 88% | 49-97 | 2150-2200 |

EST = Expressed Sequence Tag,
N/A = Not Applicable

TABLE 3

| | | Digital | | | |
|---|---|---|---|---|---|
| Seq | | Address | | | Primer |
| ID | Clone ID | (Msp1) | Gene Identity (Accession #) | Extended Primer | Seq ID |
| 1 | REC1_1 | AAGC 288 | Homo sapiens TYRO protein tyrosine kinase | GAT CGA ATC CGG AAG CCG CGC ATC ACT GAG | 86 |
| | | | binding p mRNA, and translated | | |
| | | | products (NM_003332.1) | | |
| 2 | REC1_2 | AATC 108 | NOVEL | GAT CGA ATC CGG AAT CCT GGG AGA GGC CAA | 87 |
| 3 | REC1_3 | ACGG 162 | NOVEL | GAT CGA ATC CGG ACG GGG AGG GAG CAG AGA | 88 |
| 4 | REC1_5 | AGTG 184 | NOVEL | GAT CGA ATC CGG AGT GGG AAG AGG CCT GGG | 89 |
| 5 | REC1_6 | ATCC 176 | Rabbit mRNA for ferritin light chain subunit | GAT CGA ATC CGG ATC CAG CGG CGC CGC GCG | 90 |
| | | | (X07830) | | |
| 6 | REC1_7 | CACA 282 | NOVEL | GAT CGA ATC CGG CAC ACG GGC GCA AGA AGA | 91 |
| 7 | REC1_8 | CCCG 243 | NOVEL | GAT CGA ATC CGG CCC GAT AGG TGG GTG CCC | 92 |
| 8 | REC1_10 | CTGA 390 | NOVEL | GAT CGA ATC CGG CTG AGT GAA GGA ATG ATG | 93 |
| 9 | REC1_11 | GCAT 82 | NOVEL | GAT CGA ATC CGG GCA TTA AAC ACT CTC ACA | 94 |
| 10 | REC1_12 | GCCC 232 | Cuniculus mRNA for alpha | GAT CGA ATC CGG GCC CTC CAT TGT CCA CCG | 95 |
| | | | smooth muscles actin (X60732) | | |
| 11 | REC1_13 | GCCC 216 | NOVEL | GAT CGA ATC CGG GCC CCC TCC ATC CCC AGT | 96 |
| 12 | REC1_15 | TCAG 76 | NOVEL | GAT CGA ATC CGG TCA GCC TCC CCC CCA AAA | 97 |
| 13 | REC1_16 | TCGG 195 | NOVEL | GAT CGA ATC CGG TCG GCG GCG CTC CGC GTA | 98 |
| 14 | REC1_17 | TCGT 199 | NOVEL | GAT CGA ATC CGG TCG TAT CGG CAG TGA CTT | 99 |
| 15 | REC1_18 | TGGT 241 | NOVEL | GAT CGA ATC CGG TGG TGG CCA GGA CGG AGC | 100 |
| 16 | REC1_19 | TGTT 393 | NOVEL | GAT CGA ATC CGG TGT TAC CAG TGC GAA AAG | 101 |
| 17 | REC1_20 | TTAC 210 | Cuniculus phosphofructo-1-kinase C | GAT CGA ATC CGG TTA CCA GTG TCA TTC TCC | 102 |
| | | | isozyme mRNA (U01154) | | |
| 18 | REC1_21 | TTCC 165 | Human elastin gene, exon 1 (M17282) | GAT CGA ATC CGG TTC CTC GCT CGA GCC ACT | 103 |
| 19 | REC1_22 | TGGG 164 | NOVEL | GAT CGA ATC CGG TGG GCC TTG GCT TCT CCA | 104 |
| 20 | REC1_24 | CCGG 232 | NOVEL | GAT CGA ATC CGG CCG GGC CAC GTT GGA AGC | 105 |
| 21 | REC1_28 | CGGT 101 | Rabbit mRNA for calgizzarin (D10586) | GAT CGA ATC CGG CGG TGA TAA AAC AAT AAA | 106 |
| 22 | REC1_31 | TGCA 210 | Oryctolagus cuniculus complete | GAT CGA ATC CGG TGC ACT AGC TTT AGT CTC | 107 |
| | | | mitochondrial genome (AJ001588) | | |
| 23 | REC1_33 | CCGA 96 | Oryctolagus cuniculus as1_casein gene | GAT CGA ATC CGG CCG ATG TCT CTC TCT CTC | 108 |
| | | | (M77195) | | |
| 24 | REC1_34 | CGGT 209 | NOVEL | GAT CGA ATC CGG CGG TGC CTG GAG GAC CCT | 109 |
| 25 | REC1_36 | TTAG 155 | NOVEL | GAT CGA ATC CGG TTA GCA ACC CTG AAG TGA | 110 |
| 26 | REC1_37 | TTCA 264 | R. norvegicus (Sprague Dawley) ribosomal | GAT CGA ATC CGG TTC ACA AGC ACA GGG AGA | 111 |
| | | | protein L15 mRNA (X78167) | | |
| 27 | REC1_4 | AGTA 147 | Homology to EST ut21b06.x1 | GAT CGA ATC CGG AGT ATG ATG CTC TGG ACG | 126 |
| | | | Soares_thymus_2NbMT | | |
| | | | Mus musculus cDNA clone | | |
| | | | IMAGE: 3328499 3′ | | |
| | | | similar to | | |
| | | | SW: RL2B_HUMAN P29316 | | |
| | | | 60S RIBOSOMAL | | |
| | | | PROTEIN L23A (BE691856.1) | | |
| 28 | REC1_25 | TAGC 172 | Oryctolagus cuniculus ribosomal | GAT CGA ATC CGG TAG CTC AAA AGA AAG CAA | 127 |
| | | | protein L5 homolog | | |
| | | | mRNA, partial cds (AF007880.1) | | |
| 29 | REC1_29 | TAAT 162 | Homology to Homo sapiens SPARC-like 1 | GAT CGA ATC CGG TAA TAT CAT GTG CAC ATC | 128 |
| | | | (mast9, hevin) | | |
| | | | (SPARCL1), mRNA (NM_004684.1) | | |
| 30 | REC1_32 | GATA 202 | Oryctolagus cuniculus gene encoding | GAT CGA ATC CGG GAT ACG TAC CTG GCT CCT | 129 |
| | | | ileal sodium-dependent bile acid | | |
| | | | transporter (AJ002005.1) | | |
| 31 | REC1_35 | CTTA 195 | Oryctolagus cuniculus mitochondrial | GAT CGA ATC CGG CTT ACA AGA CCA GAG TAA | 130 |
| | | | cytB and tRNA-Thr | | |
| | | | genes for cytochrome b and transfer RNA-Thr | | |
| | | | (AJ243197.1) | | |
| 32 | REC1_38 | GTGC 100 | NOVEL | GAT CGA ATC CGG GTG GGC ATT TTA TTT GTG | 131 |
| 33 | REC1_39 | CTGG 429 | Homologous to Mus musculus follistatin-like 2 | GAT CGA ATC CGG CTG GGT GCT GGT GTC GCC | 132 |
| | | | (Fstl2) mRNA (NM_008048.1) | | |
| 34 | REC1_40 | CTGC 130 | NOVEL | GAT CGA ATC CGG CTG CAG AAG CAA GAA CCC | 133 |
| 35 | REC1_42 | ACAC 268 | Homology to Human 18S rRNA | GAT CGA ATC CGG ACA CGG ACA GGA TTG ACA | 134 |
| | | | gene (K03432.1) | | |
| 36 | REC1_43 | AGGC 164 | Homology to human ribosomal | GAT CGA ATC CGG AGG CAG GCC GAG AAG AAC | 135 |
| | | | protein L13a (RPL13A) (NM_012423.1) | | |
| 37 | REC1_44 | AGGT 157 | NOVEL | GAT CGA ATC CGG AGG TCG ACA GGA CCC AGA | 136 |
| 38 | REC1_45 | CAGT 143 | Oryctolagus cuniculus cGMP-gated | GAT CGA ATC CGG CAG TAG CAG CCA TTT GGG | 137 |
| | | | potassium channel | | |
| | | | (Kcnl) gene, 5UTR, promoter | | |
| | | | and enhancer (U38183.1) | | |
| 39 | REC1_46 | CGCT 219 | Homology to Homo sapiens Arp2/3 protein | GAT CGA ATC CGG CGC TTG ACG GTT GTC ACA | 138 |
| | | | complex subunit p16-Arc (ARC16) mRNA, | | |
| | | | complete cds (AF006088.1) | | |
| 40 | REC1_47 | GATC 225 | Homology to EST ia05c08.x1 Human | GAT CGA ATC CGG GAT CCT GTC AGA AGG GGG | 139 |
| | | | Pancreatic Islets Homo sapiens cDNA | | |
| | | | 3 similar to gb: X62320 | | |
| | | | GRANULINS PRECURSOR | | |
| | | | (HUMAN); gb: M86736 | | |
| | | | Mouse acrogranin mRNA, | | |
| | | | Pancreatic Islets complete cds (MOUSE) | | |
| | | | Pancreatic Islets (AW583467.1) | | |
| 41 | REC1_48 | TTAC 162 | Homologous to Homo sapiens CGI-134 | GAT CGA ATC CGG TTA CAT ACA AGG ATC CTG | 140 |
| | | | protein mRNA, complete cds. (AF151892.1) | | |
| 42 | REC1_49 | TCCG 167 | Homology to Rat mRNA for ribosomal | GAT CGA ATC CGG TCC GTG GCT CGC ATC GCC | 141 |
| | | | protein L7a (X15013.1) | | |
| 43 | REC1_50 | AGGA 205 | Homology to human CGI-71 protein mRNA | GAT CGA ATC CGG AGG AGC CCA AGT GGG TAG | 142 |
| | | | (AF151829.1) | | |
| 44 | REC1_51 | GACT 89 | NOVEL | GAT CGA ATC CGG GAC TGT GAG AAA TAA ACG | 143 |
| 45 | REC1_52 | CGGG 293 | Homology to Homo sapiens ribosomal | GAT CGA ATC CGG CGG GCA CAG GCC CAA AGG | 144 |
| | | | protein S10 (RPS10) mRNA | | |
| | | | (NM_001014.1) | | |
| 46 | REC1_53 | CCCC 194 | NOVEL | GAT CGA ATC CGG CCC CGC CCT GCT TCC ACG | 145 |
| 47 | REC1_54 | TGCT 452 | Homology to Homo sapiens mRNA | GAT CGA ATC CGG TGC TAC TTG AGA CAA AAG | 146 |
| | | | for alpha-tropomyosin (3′ end) (AJ000147.1) | | |
| 48 | REC1_55 | ATTT 289 | Homology to Homo sapiens four and a | GAT CGA ATC CGG ATT TCC ACC TAC CAC TTC | 147 |
| | | | half LIM domains 1 (FHL1), mRNA | | |
| | | | (XM_010369.1) | | |
| 49 | REC1_56 | TGAC 197 | Homology to Homo sapiens ribosomal | GAT CGA ATC CGG TGA CAT TCC TGG CGT CCG | 148 |
| | | | protein S23 (RPS23) mRNA | | |
| | | | (NM_001025.1) | | |
| 50 | REC1_57 | GTGA 274 | Homology to Homo sapiens ribosomal | GAT CGA ATC CGG GTG ACC AGA CAG GCC ATG | 149 |
| | | | protein L14 (RPL14) mRNA | | |
| | | | (NM_003973.1) | | |
| 51 | REC1_58 | AGTT 124 | NOVEL | GAT CGA ATC CGG AGT TAT ACC ACA GTG ATT | 150 |
| 52 | REC1_59 | ATTA 147 | Homology to Homo sapiens genomic DNA, | GAT CGA ATC CGG ATT ATC ATG CTC TGG ACG | 151 |
| | | | chromosome 21q, section 68/105 (AP001724.1) | | |
| 53 | REC1_60 | GCGT 178 | EST hv32a11.x1 NCI_CGAP_Lu24 | GAT CGA ATC CGG GCG TTG TAC TGC GTT TGG | 152 |
| | | | Homo sapiens cDNA clone | | |
| | | | IMAGE: 3175100 (BE217941.1) | | |
| 54 | REC1_61 | ACCA 174 | NOVEL | GAT CGA ATC CGG ACC ACG GTG CCA CAT TGC | 153 |
| 55 | REC1_62 | GAGA 168 | Homo sapiens zyxin (ZYX) | GAT CGA ATC CGG GAG AGG TCC TGA CTC TCT | 154 |
| | | | mRNA (NM_003461.1) | | |

EST = Expressed Sequence Tag,
N/A = Not Applicable

TABLE 4
VERIFIED CANDIDATE MATCHES

| Digital | | |
|---|---|---|
| Address | | |
| (Msp1) | Gene Identity (Accession #) | Extended Primer |
| ATAC 176 | Rabbit mRNA for matrix metalloproteinase 9, complete cds | GAT CGA ATC CGG ATA CAA ACT GGG CGT TTG (SEQ ID NO: 112) |
| | (D26514) | |
| CAGT 170 | Oryctolagus cuniculus ubiquitin-activating | GAT CGA ATC CGG CAG TGG CCC ACC TAG CCA (SEQ ID NO: 113) |
| | enzyme E1 mRNA, complete cds (U58653) | |
| CCGA 259 | Rabbit IgA-alpha chain constant region mRNA fragment | GAT CGA ATC CGG CCG AGG ACT GGA ACC AGG (SEQ ID NO: 114) |
| | (X00353) | |
| GTCC 111 | Rabbit mRNA for protein phosphatase-1 catalytic subunit | GAT CGA ATC CGG GTC CCA GGG GAG CCC GTC (SEQ ID NO: 115) |
| | (EC3.1.3.-) (Y00701) | |
| GTTG 116 | Oryctolagus cuniculus unknown mRNA, partial | GAT CGA ATC CGG GTT GTG GGC TGT GAC TTC (SEQ ID NO: 116) |
| | cds (AF011560) | |
| TATT 315 | Oryctolagus cuniculus 1-caldesmon mRNA, | GAT CGA ATC CGG TAT TTG GAT GGA ATA AAT (SEQ ID NO: 117) |
| | complete cds (L37147) | |
| CCTG 172 | Rabbit mRNA for interleukin 1 receptor | GAT CGA ATC CGG CCT GGC CCT GAG CAA GTA (SEQ ID NO: 118) |
| | antagonist, complete cds (D21832.1) | |

TABLE 24
RT-PCR Validation

| | | Digital | | | |
|---|---|---|---|---|---|
| Seq | | Address | | | Validation |
| ID | Clone ID | (Msp1) | Database Match (Accession #) | RT-PCR Fluorimetry Results | Method |
| 1 | REC1_1 | AAGC 288 | Homo sapiens TYRO protein tyrosine kinase binding | See FIG. 6 and Table 5 | RT-PCR |
| | | | p mRNA, and translated products (NM_003332.1) | | |
| 2 | REC1_2 | AATC 108 | NOVEL | See FIG. 7 and Table 6 | RT-PCR |
| 3 | REC1_3 | ACGG 162 | NOVEL | See FIG. 8 and Table 7 | RT-PCR |
| 4 | REC1_5 | AGTG 184 | NOVEL | See FIG. 15 and Table 14 | RT-PCR |
| 5 | REC1_6 | ATCC 176 | Rabbit mRNA for ferritin light chain subunit | See FIG. 11 and Table 10 | RT-PCR |
| | | | (X07830) | | |
| 6 | REC1_7 | CACA 282 | NOVEL | See FIG. 14 and Table 13 | RT-PCR |
| 7 | REC1_8 | CCCG 243 | NOVEL | See FIG. 9 and Table 8 | RT-PCR |
| 8 | REC1_10 | CTGA 390 | NOVEL | See FIG. 10 and Table 9 | RT-PCR |
| 10 | REC1_12 | GCCC 232 | Cuniculus mRNA for alpha smooth muscle actin | See FIG. 21 and Table 20 | RT-PCR |
| | | | (X60732) | | |
| 11 | REC1_13 | GCCC 216 | NOVEL | See FIG. 12 and Table 11 | RT-PCR |
| 13 | REC1_16 | TCGG 195 | NOVEL | See FIG. 16 and Table 15 | RT-PCR |
| 14 | REC1_17 | TCGT 199 | NOVEL | See FIG. 17 and Table 16 | RT-PCR |
| 15 | REC1_18 | TGGT 241 | NOVEL | See FIG. 13 and Table 12 | RT-PCR |
| 16 | REC1_19 | TGTT 393 | NOVEL | See FIG. 18 and Table 17 | RT-PCR |
| 17 | REC1_20 | TTAC 210 | Cuniculus phosphofructo-1-kinase C isozyme mRNA | See FIG. 19 and Table 18 | RT-PCR |
| | | | (U01154) | | |
| 18 | REC1_21 | TTCC 165 | Human elastin gene, exon 1 (M17282) | See FIG. 20 and Table 19 | RT-PCR |
| 19 | REC1_22 | TGGG 164 | NOVEL | See FIG. 22 and Table 21 | RT-PCR |
| 20 | REC1_24 | CCGG 232 | NOVEL | See FIG. 23 and Table 22 | RT-PCR |
| 25 | REC1_36 | TTAG 155 | NOVEL | See FIG. 24 and Table 23 | RT-PCR |

RT-PCR = Reverse Transcriptase-Polymerase Chain Reaction

TABLE 25
RT-PCR Validation Primers

| | Digital | | | Seq | | Seq |
|---|---|---|---|---|---|---|
| | Address | Database Match | | ID | | ID |
| Clone ID | (Msp1) | (Accession #) | 5′ RT-PCR Primer | NO: | 3′ RT-PCR Primer | NO: |
| REC1_1 | AAGC 288 | Homo sapiens TYRO | TCC AGG GTC AGA GGT CGA TGT | 155 | GCA GCA ACA | 156 |
| | | protein tyrosine | GTA GAG GGG AGA GGT | | | |
| | | kinase binding p | | | | |
| | | mRNA, and | | | | |
| | | translated products | | | | |
| | | (NM_003332.1) | | | | |
| REC1_2 | AATC 108 | NOVEL | AAT CCT GGG AGA GGC CAA G | 157 | TTG AAG CAA AAG CGC TCT CTT | 158 |
| REC1_3 | ACGG 162 | NOVEL | GAC GGG GAG GGA GCA GAG | 159 | TGC TCT CCT TGG AGG ATC ACA | 160 |
| REC1_5 | AGTG 184 | NOVEL | GAG GCC TGG GCT CAA G | 161 | GAT GAT TAA AAT GTC CCC AGC | 162 |
| REC1_6 | ATCC 176 | Rabbit mRNA for | CGC TCT CCT GCC AGC C | 163 | TTC GCT GCA GCA AGA AAC TTT | 164 |
| | | ferritin light chain | | | | |
| | | subunit (X07830) | | | | |
| REC1_7 | CACA 282 | NOVEL | CGG GCG CAA GAA GAC A | 165 | TGC TAG CCC CAA AGA TAA AAA | 166 |
| REC1_8 | CCCG 243 | NOVEL | GTG GGT GCC CTG GCT G | 167 | CCA CTT TCT ACC CCA GGC TCC | 168 |
| REC1_10 | CTGA 390 | NOVEL | CAC CTG CCC TCA GAG TTG ACA | 169 | GGC AGA CAG GGT TCA CCA CA | 170 |
| REC1_12 | GCCC 232 | Cuniculus mRNA | CAC CGC AAA TGC TTC TA | 171 | TTT GTG GTA GCA TAT TTC AGA | 172 |
| | | for alpha smooth | | | | |
| | | muscle actin (X60732) | | | | |
| REC1_13 | GCCC 216 | NOVEL | CCC CCT CCA TCC CCA GTG A | 173 | CCT CAC ACG TGG GTG GCT CAC | 174 |
| REC1_16 | TCGG 195 | NOVEL | CGT AGA CTC GCC GAG GTT TTC | 175 | TTT TTG GCG TCT CAC ACT TCA | 176 |
| REC1_17 | TCGT 199 | NOVEL | GGT CGT ATC GGC AGT GAC TT | 177 | CTG GGA AAA TAA GCC TCA TTG | 178 |
| REC1_18 | TGGT 241 | NOVEL | ACG GAG CCG CTC AGG GA | 179 | CCC TCG GCA CCA GAG TAG GAA | 180 |
| REC1_19 | TGTT 393 | NOVEL | CCG GTG TTA CCA GTG CGA AAA | 181 | GGT CTG CCC CAT CCT TGT CAG | 182 |
| REC1_20 | TTAC 210 | Cuniculus | CGG TTA CCA GTG TCA TTC TCC | 183 | GGG GTT TAG GCC AAA TGT TTA | 184 |
| | | phosphofructo-1- | | | | |
| | | kinase C | | | | |
| | | isozyme mRNA | | | | |
| | | (U01154) | | | | |
| REC1_21 | TTCC 165 | Human elastin gene, | CCT CGC TCG AGC CAC T | 185 | TCT ATC ACA GCG AGA CAC AGA | 186 |
| | | exon 1 (M17282) | | | | |
| REC1_22 | TGGG 164 | NOVEL | GGG CCT TGG CTT CTC C | 187 | ATG CAG GTC AGA AAG AAT TGG | 188 |
| REC1_24 | CCGG 232 | NOVEL | CGA ACA CTG GAA TGC ATA TA | 189 | AAC CCT TTA TTA CAA GTC ACG | 190 |
| REC1_36 | TTAG 155 | NOVEL | GCA ACC CTG AAG TGA T | 191 | AAT TCT CAT TCA GTA AAG ATT | 192 |

We claim:

1. An isolated nucleic acid molecule comprising a polynucleotide chosen from the group consisting of
SEQ ID NO:1, SEQ ID NO:2, SEQ ID NO:3, SEQ ID NO:4, SEQ ID NO:5, SEQ ID NO:6,
SEQ ID NO:7, SEQ ID NO:8, SEQ ID NO:9, SEQ ID NO:10, SEQ ID NO:11, SEQ ID NO:12,
SEQ ID NO:13, SEQ ID NO:14, SEQ ID NO:15, SEQ ID NO:16, SEQ ID NO:17, SEQ ID NO:18,
SEQ ID NO:19, SEQ ID NO:20, SEQ ID NO:21, SEQ ID NO:22, SEQ ID NO:23, SEQ ID NO:24,
SEQ ID NO:25, SEQ ID NO:26, SEQ ID NO:27, SEQ ID NO:28, SEQ ID NO:29, SEQ ID NO:30,
SEQ ID NO:31, SEQ ID NO:32, SEQ ID NO:33, SEQ ID NO:34, SEQ ID NO:35, SEQ ID NO:36,
SEQ ID NO:37, SEQ ID NO:38, SEQ ID NO:39, SEQ ID NO:40, SEQ ID NO:41, SEQ ID NO:42,
SEQ ID NO:43, SEQ ID NO:44, SEQ ID NO:45, SEQ ID NO:46, SEQ ID NO:47, SEQ ID NO:48,
SEQ ID NO:49, SEQ ID NO:50, SEQ ID NO:51, SEQ ID NO:52, SEQ ID NO:53, SEQ ID NO:54
and SEQ ID NO:55.

2. An isolated polypeptide encoded by a polynucleotide chosen from the group consisting of
SEQ ID NO:1, SEQ ID NO:2, SEQ ID NO:3, SEQ ID NO:4, SEQ ID NO:5, SEQ ID NO:6,
SEQ ID NO:7, SEQ ID NO:8, SEQ ID NO:9, SEQ ID NO:10, SEQ ID NO:11, SEQ ID NO:12,
SEQ ID NO:13, SEQ ID NO:14, SEQ ID NO:15, SEQ ID NO:16, SEQ ID NO:17, SEQ ID NO:18,
SEQ ID NO:19, SEQ ID NO:20, SEQ ID NO:21, SEQ ID NO:22, SEQ ID NO:23, SEQ ID NO:24,
SEQ ID NO:25, SEQ ID NO:26, SEQ ID NO:27, SEQ ID NO:28, SEQ ID NO:29, SEQ ID NO:30,
SEQ ID NO:31, SEQ ID NO:32, SEQ ID NO:33, SEQ ID NO:34, SEQ ID NO:35, SEQ ID NO:36,
SEQ ID NO:37, SEQ ID NO:38, SEQ ID NO:39, SEQ ID NO:40, SEQ ID NO:41, SEQ ID NO:42,
SEQ ID NO:43, SEQ ID NO:44, SEQ ID NO:45, SEQ ID NO:46, SEQ ID NO:47, SEQ ID NO:48,
SEQ ID NO:49, SEQ ID NO:50, SEQ ID NO:51, SEQ ID NO:52, SEQ D NO:53, SEQ ID NO:54
and SEQ ID NO:55.

3. An isolated nucleic acid molecule comprising a polynucleotide at least 95% identical to the isolated nucleic acid molecule of claim 1.

4. An isolated nucleic acid molecule at least ten bases in length that is hybridizable to the isolated nucleic acid molecule of claim 1 under stringent conditions.

5. An isolated nucleic acid molecule encoding the polypeptide of claim 2.

6. An isolated nucleic acid molecule encoding a fragment of the polypeptide of claim 2.

7. An isolated nucleic acid molecule encoding a polypeptide epitope of the polypeptide of claim 2.

8. The polypeptide of claim 2 wherein the polypeptide has biological activity.

9. An isolated nucleic acid encoding a species homologue of the polypeptide of claim 2.

10. The isolated nucleic acid molecule of claim 1, wherein the nucleotide sequence comprises SEQ ID NO:1.

11. The isolated nucleic acid molecule of claim 1, wherein the nucleotide sequence comprises SEQ ID NO:2.

12. The isolated nucleic acid molecule of claim 1, wherein the nucleotide sequence comprises SEQ ID NO:3.

13. The isolated nucleic acid molecule of claim 1, wherein the nucleotide sequence comprises SEQ ID NO:4.

14. The isolated nucleic acid molecule of claim 1, wherein the nucleotide sequence comprises SEQ ID NO:5.

15. The isolated nucleic acid molecule of claim 1, wherein the nucleotide sequence comprises SEQ ID NO:6.

16. The isolated nucleic acid molecule of claim 1, wherein the nucleotide sequence comprises SEQ ID NO:7.

17. The isolated nucleic acid molecule of claim 1, wherein the nucleotide sequence comprises SEQ ID NO:8.

18. The isolated nucleic acid molecule of claim 1, wherein the nucleotide sequence comprises SEQ ID NO:9.

19. The isolated nucleic acid molecule of claim 1, wherein the nucleotide sequence comprises SEQ ID NO:10.

20. The isolated nucleic acid molecule of claim 1, wherein the nucleotide sequence comprises SEQ ID NO:11.

21. The isolated nucleic acid molecule of claim 1, wherein the nucleotide sequence comprises SEQ ID NO:12.

22. The isolated nucleic acid molecule of claim 1, wherein the nucleotide sequence comprises SEQ ID NO:13.

23. The isolated nucleic acid molecule of claim 1, wherein the nucleotide sequence comprises SEQ ID NO:14.

24. The isolated nucleic acid molecule of claim 1, wherein the nucleotide sequence comprises SEQ ID NO:15.

25. The isolated nucleic acid molecule of claim 1, wherein the nucleotide sequence comprises SEQ ID NO:16.

26. The isolated nucleic acid molecule of claim 1, wherein the nucleotide sequence comprises SEQ ID NO:17.

27. The isolated nucleic acid molecule of claim 1, wherein the nucleotide sequence comprises SEQ ID NO:18.

28. The isolated nucleic acid molecule of claim 1, wherein the nucleotide sequence comprises SEQ ID NO:19.

29. The isolated nucleic acid molecule of claim 1, wherein the nucleotide sequence comprises SEQ ID NO:20.

30. The isolated nucleic acid molecule of claim 1, wherein the nucleotide sequence comprises SEQ ID NO:21.

31. The isolated nucleic acid molecule of claim 1, wherein the nucleotide sequence comprises SEQ ID NO:22.

32. The isolated nucleic acid molecule of claim 1, wherein the nucleotide sequence comprises SEQ ID NO:23.

33. The isolated nucleic acid molecule of claim 1, wherein the nucleotide sequence comprises SEQ ID NO:24.

34. The isolated nucleic acid molecule of claim 1, wherein the nucleotide sequence comprises SEQ ID NO:25.

35. The isolated nucleic acid molecule of claim 1, wherein the nucleotide sequence comprises SEQ ID NO:26.

26. The isolated nucleic acid molecule of claim 1, wherein the nucleotide sequence comprises SEQ ID NO:27.

37. The isolated nucleic acid molecule of claim 1, wherein the nucleotide sequence comprises SEQ ID NO:28.

38. The isolated nucleic acid molecule of claim 1, wherein the nucleotide sequence comprises SEQ ID NO:29.

39. The isolated nucleic acid molecule of claim 1, wherein the nucleotide sequence comprises SEQ ID NO:30.

40. The isolated nucleic acid molecule of claim 1, wherein the nucleotide sequence comprises SEQ ID NO:31.

41. The isolated nucleic acid molecule of claim 1, wherein the nucleotide sequence comprises SEQ ID NO:32.

42. The isolated nucleic acid molecule of claim 1, wherein the nucleotide sequence comprises SEQ ID NO:33.

43. The isolated nucleic acid molecule of claim 1, wherein the nucleotide sequence comprises SEQ ID NO:34.

44. The isolated nucleic acid molecule of claim 1, wherein the nucleotide sequence comprises SEQ ID NO:35.

45. The isolated nucleic acid molecule of claim 1, wherein the nucleotide sequence comprises SEQ ID NO:36.

46. The isolated nucleic acid molecule of claim 1, wherein the nucleotide sequence comprises SEQ ID NO:37.

47. The isolated nucleic acid molecule of claim 1, wherein the nucleotide sequence comprises SEQ ID NO:38.

48. The isolated nucleic acid molecule of claim 1, wherein the nucleotide sequence comprises SEQ ID NO:39.

49. The isolated nucleic acid molecule of claim 1, wherein the nucleotide sequence comprises SEQ ID NO:40.

50 The isolated nucleic acid molecule of claim 1, wherein the nucleotide sequence comprises SEQ ID NO:41.

51. The isolated nucleic acid molecule of claim 1, wherein the nucleotide sequence comprises SEQ ID NO:42.

52. The isolated nucleic acid molecule of claim 1, wherein the nucleotide sequence comprises SEQ ID NO:43.

53. The isolated nucleic acid molecule of claim 1, wherein the nucleotide sequence comprises SEQ ID NO:44.

54. The isolated nucleic acid molecule of claim 1, wherein the nucleotide sequence comprises SEQ ID NO:45.

55. The isolated nucleic acid molecule of claim 1, wherein the nucleotide sequence comprises SEQ ID NO:46.

56. The isolated nucleic acid molecule of claim 1, wherein the nucleotide sequence comprises SEQ ID NO:47.

57. The isolated nucleic acid molecule of claim 1, wherein the nucleotide sequence comprises SEQ ID NO:48.

58. The isolated nucleic acid molecule of claim 1, wherein the nucleotide sequence comprises SEQ ID NO:49.

59. The isolated nucleic acid molecule of claim 1, wherein the nucleotide sequence comprises SEQ ID NO:50.

60. The isolated nucleic acid molecule of claim 1, wherein the nucleotide sequence comprises SEQ ID NO:51.

61. The isolated nucleic acid molecule of claim 1, wherein the nucleotide sequence comprises SEQ ID NO:52.

62. The isolated nucleic acid molecule of claim 1, wherein the nucleotide sequence comprises SEQ ID NO:53.

63. The isolated nucleic acid molecule of claim 1, wherein the nucleotide sequence comprises SEQ ID NO:54.

64. The isolated nucleic acid molecule of claim 1, wherein the nucleotide sequence comprises SEQ ID NO:55.

65. The isolated nucleic acid molecule of claim 1, wherein the nucleotide sequence comprises sequential nucleotide deletions from either the 5′ end or the 3′end.

66. A recombinant vector comprising the isolated nucleic acid molecule of claim 1.

67. A recombinant host cell comprising the isolated nucleic acid molecule of claim 1.

68. A method of making the recombinant host cell of claim 67.

69. The recombinant host cell of claim 67 comprising vector sequences.

70. The isolated polypeptide of claim 2, wherein the isolated polypeptide comprises sequential amino acid deletions from either the C-terminus or the N-terminus.

71. An isolated antibody that binds specifically to the isolated polypeptide of claim 2.

72. The isolated antibody of claim 71 wherein the antibody is a monoclonal antibody.

73. The isolated antibody of claim 72 wherein the antibody is a polyclonal antibody.

74. A recombinant host cell that expresses the isolated polypeptide of claim 2.

75. An isolated polypeptide produced by the steps of:
(a) culturing the recombinant host cell of claim 14 under conditions such that said polypeptide is expressed; and
(b) isolating the polypeptide.

76. A method for preventing, treating, modulating, or ameliorating a medical condition, comprising administering to a mammalian subject a therapeutically effective amount of the polypeptide of claim 2 or the polynucleotide of claim 1.

77. The method of claim 76 wherein the medical condition is atherosclerosis.

78. A method for preventing, treating, modulating, or ameliorating a medical condition comprising administering to a mammalian subject a therapeutically effective amount of the antibody of claim 71.

79. The method of claim 78 wherein the medical condition is atherosclerosis.

80. A method of diagnosing a pathological condition or a susceptibility to a pathological condition in a subject comprising:
(a) determining the presence or absence of a mutation in the polynucleotide of claim 1; and
(b) diagnosing a pathological condition or a susceptibility to a pathological condition based on the presence or absence of said mutation.

81. The method of claim 80 wherein the pathological condition is atherosclerosis.

82. A method of diagnosing a pathological condition or a susceptibility to a pathological condition in a subject comprising detecting an alteration in expression of a polypeptide encoded by the polynucleotide of claim 1, wherein the presence of an alteration in expression of the polypeptide is indicative of the pathological condition or susceptibility to the pathological condition.

83. The method of claim 82 wherein the alteration in expression is an increase in the amount of expression or a decrease in the amount of expression.

84. The method of claim 82 wherein the pathological condition is atherosclerosis.

85. The method of claim 84 wherein the method further comprises the steps of:
obtaining a first biological sample from a patient suspected of having atherosclerosis and obtaining a second sample from a suitable comparable control source;
(a) determining the amount of at least one polypeptide encoded by a polynucleotide of claim 1 in the first and second sample; and
(b) comparing the amount of the polypeptide in the first and second samples;
wherein a patient is diagnosed as having atherosclerosis if the amount of the polypeptide in the first sample is greater than or less than the amount of the polypeptide in the second sample.

86. The use of the polynucleotide of claim 1 or polypeptide of claim 2 for the manufacture of a medicament for the treatment of atherosclerosis.

87. The use of the antibody of claim 71 for the manufacture of a medicament for the treatment of atherosclerosis.

88. A method for identifying a binding partner to the polypeptide of claim 2 comprising:
(a) contacting the polypeptide of claim 2 with a binding partner; and
(b) determining whether the binding partner effects an activity of the polypeptide.

89. The gene corresponding to the cDNA sequence of the isolated nucleic acid of claim 1.

90. A method of identifying an activity of an expressed polypeptide in a biological assay, wherein the method comprises:
(a) expressing the polypeptide of claim 2 in a cell;
(b) isolating the expressed polypeptide;
(c) testing the expressed polypeptide for an activity in a biological assay; and
(d) identifying the activity of the expressed polypeptide based on the test results.

91. A substantially pure isolated DNA molecule suitable for use as a probe for genes regulated in atherosclerosis, chosen from the group consisting of the DNA molecules identified in Table 1, having a 5′ partial nucleotide sequence and length as described by their digital address, and having a characteristic regulation pattern in atherosclerosis.

92. A kit for detecting the presence of the polypeptide of the claim 2 in a mammalian tissue sample comprising a first antibody which immunoreacts with a mammalian protein encoded by a gene corresponding to the polynucleotide of claim 1 or with a polypeptide encoded by the polynucleotide of claim 2 in an amount sufficient for at least one assay and suitable packaging material.

93. A kit of claim 92 further comprising a second antibody that binds to the first antibody.

94. The kit of claim 93 wherein the second antibody is labeled.

95. The kit of claim 94 wherein the label comprises enzymes, radioisotopes, fluorescent compounds, colloidal metals, chemiluminescent compounds, phosphorescent compounds, or bioluminescent compounds.

96. A kit for detecting the presence of a genes encoding an protein comprising a polynucleotide of claim 1, or fragment thereof having at least 10 contiguous bases, in an amount sufficient for at least one assay, and suitable packaging material.

97. A method for detecting the presence of a nucleic acid encoding a protein in a mammalian tissue sample, comprising the steps of:
(a) hybridizing a polynucleotide of claim 1 or fragment thereof having at least 10 contiguous bases, with the nucleic acid of the sample; and
(b) detecting the presence of the hybridization product.

98. A method of diagnosing or montoring the presence of development of atherosclerosis in hypercholesterolemia in a subject comprising detecting an alteration in expression of at least one polypeptide encoded by at least one polynucleotide selected from the group consisting of SEQ ID NO:1, SEQ ID NO:2, SEQ ID NO:3, SEQ ID NO:5, SEQ ID NO:7, SEQ ID NO:8, SEQ ID NO:9, SEQ ID NO:11 and SEQ ID NO:21.

99. A method of diagnosing or montoring the presence of development of atherosclerosis in hypercholesterolemia in a subject comprising detecting an alteration in expression of at least one polypeptide encoded by at least one polynucleotide selected from the group consisting of SEQ ID NO:4, SEQ ID NO:6, SEQ ID NO:13, SEQ ID NO:14, SEQ ID NO:16, SEQ ID NO:17 and SEQ ID NO:18.

100. A method of diagnosing or montoring the presence of development of atherosclerosis in hypercholesterolemia in a subject comprising comparing an alteration in expression of at least one polypeptide encoded by at least one polynucleotide selected from the group consisting of SEQ ID NO:1, SEQ ID NO:2, SEQ ID NO:3, SEQ ID NO:5, SEQ ID NO:7, SEQ ID NO:8, SEQ ID NO:9, SEQ ID NO:11 and SEQ ID NO:21 to an alteration in expression of at least one polypeptide encoded by at least one polynucleotide selected from the group consisting of SEQ ID NO:4, SEQ ID NO:6, SEQ ID NO:13, SEQ ID NO:14, SEQ ID NO:16, SEQ ID NO:17 and SEQ ID NO:18.

101. A method of diagnosing or montoring the effects of treating a subject with a dihydropyridine calcium antagonist comprising detecting an alteration in expression of at least one polypeptide encoded by at least one polynucleotide selected from the group consisting of
SEQ ID NO:19, SEQ ID NO:20, SEQ ID NO:21,
SEQ ID NO:22, SEQ ID NO:23 and SEQ ID NO:24.

101. The method of claim 100 wherein the dihydropyridine calcium antagonist is lercanidipine.
